(19) United States
(12) Patent Application Publication
(10) Pub. No.: US 2026/0007723 A1
(43) Pub. Date: Jan. 8, 2026

(54) GLYCOTARGETING THERAPEUTICS

(71) Applicants: École Polytechnique Fédérale de Lausanne (EPFL), Lausanne (CH); Anokion SA, Ecublens (CH)

(72) Inventors: Jeffrey A. Hubbell, New York, NY (US); Stephan Kontos, Belmont, CA (US); Kristen Marie Lorentz, Belmont, CA (US); David Scott Wilson, Baltimore, MD (US); Shuning Gai, Menlo Park, CA (US)

(21) Appl. No.: 19/056,135

(22) Filed: Feb. 18, 2025

Related U.S. Application Data
(63) Continuation of application No. 17/810,574, filed on Jul. 1, 2022, now abandoned.
Application No. 17/810,574 is a continuation of application No. 16/723,774, filed on Dec. 20, 2019, now Pat. No. 11,666,638.
Application No. 16/723,774 is a continuation of application No. 14/627,297, filed on Feb. 20, 2015, now Pat. No. 10,821,157.
(60) Provisional application No. 61/942,942, filed on Feb. 21, 2014.

(51) Int. Cl. A61K 38/38; A61K 38/28; A61K 39/00; A61K 39/35; A61K 39/385; A61K 47/54

(52) U.S. Cl. CPC A61K 38/38; A61K 38/28; A61K 39/001; A61K 39/35; A61K 39/385; A61K 47/549; A61K 47/555; A61K 2039/555; A61K 2039/577; A61K 2039/60; A61K 2039/6093; A61K 2039/627; C07K 2319/01

(57) ABSTRACT

Glycotargeting therapeutics are useful in the treatment of transplant rejection, autoimmune disease, food allergy, and immune response against a therapeutic agent.

### CROSS-REFERENCE TO RELATED APPLICATION

This application is a continuation of co-pending U.S. application Ser. No. 17/810,574, filed Jul. 1, 2022, which is a continuation of Ser. No. 16/723,774, filed Dec. 20, 2019, now U.S. Pat. No. 11,666,638, which is a continuation of U.S. application Ser. No. 14/627,297, filed Feb. 20, 2015, now U.S. Pat. No. 10,821,157, which claims the benefit of priority to U.S. Ser. No. 61/942,942 filed Feb. 21, 2014, the disclosures of each of which are hereby incorporated by reference in their entireties.

### FIELD OF THE DISCLOSURE

The field of the disclosure relates to pharmaceutically acceptable compositions that are useful in the treatment of transplant rejection, autoimmune disease, food allergy, and immune response against a therapeutic agent.

### INCORPORATION BY REFERENCE OF MATERIAL IN ASCII FILE

This application incorporates by reference the material contained in the Sequence Listing XML file being submitted concurrently herewith: File name: Sequence_Listing_95611.00937_ST26.xml; created Feb. 18, 2025, 56747 bytes in size.

### BACKGROUND OF THE DISCLOSURE

Applications US 2012/0039989, US 2012/0178139 and WO 2013/121296 describe the targeting of antigens to erythrocytes to take advantage of the erythrocytes' role in antigen presentation for tolerization. Notwithstanding the positive results generated to date with this approach, the possibility of alternative approaches has remained of interest.

### SUMMARY OF THE DISCLOSURE

An aspect of the disclosure provides a composition comprising a compound of Formula 1:

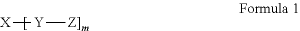

where:
m is an integer from about 1 to 100;
X comprises an antigen against which a patient develops an unwanted immune response, or a tolerogenic portion thereof; or
X comprises an antibody, antibody fragment or ligand that specifically binds a circulating protein or peptide or antibody, which circulating protein or peptide or antibody is causatively involved in transplant rejection, immune response against a therapeutic agent, autoimmune disease, hypersensitivity and/or allergy;
Y comprises a linker moiety; and
Z comprises a liver-targeting moiety.

Z can also comprise galactose, galactosamine or N-acetylgalactosamine, for example, conjugated at its C1, C2 or C6 to Y.

Y can be Y is selected from N-hydroxysuccinamidyl linkers, malaemide linkers, vinylsulfone linkers, pyridyl di-thiol-poly(ethylene glycol) linkers, pyridyl di-thiol linkers, n-nitrophenyl carbonate linkers, NHS-ester linkers, and nitrophenoxy poly(ethylene glycol) ester linkers.

Y can also comprise: an antibody, antibody fragment, peptide or other ligand that specifically binds X; a disulfanyl ethyl ester; a structure represented by one of Formulae Ya to Yp:

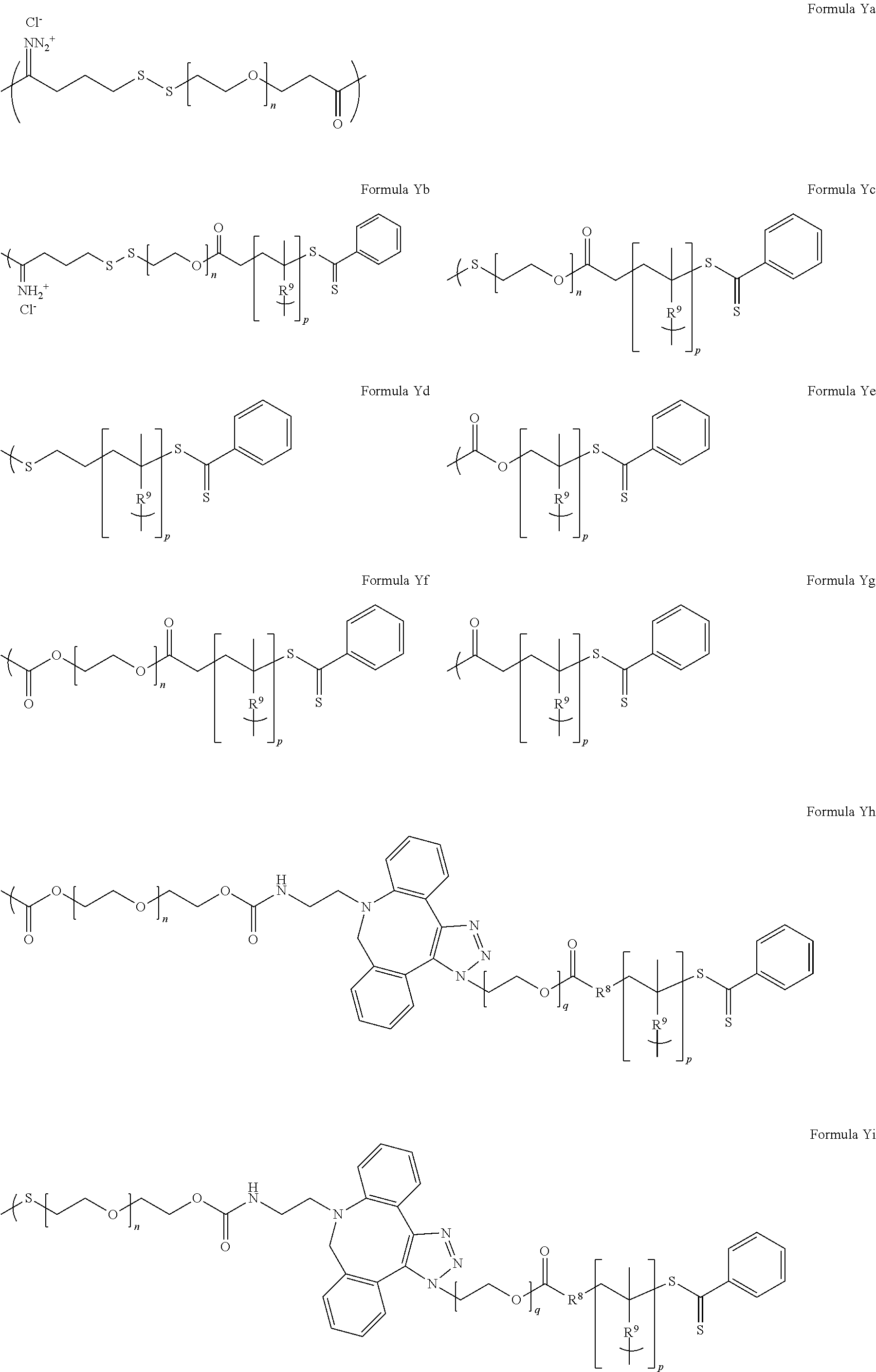

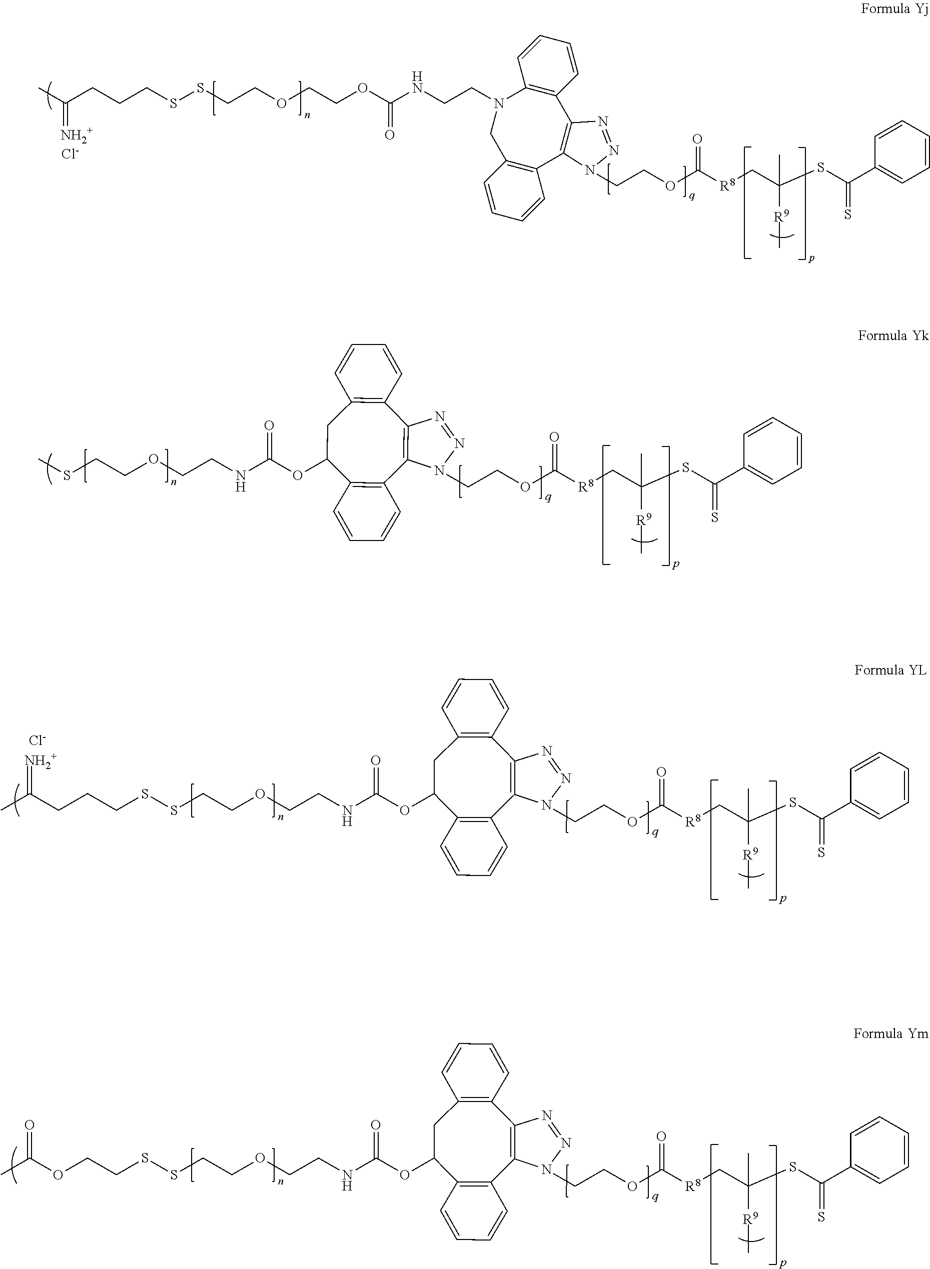

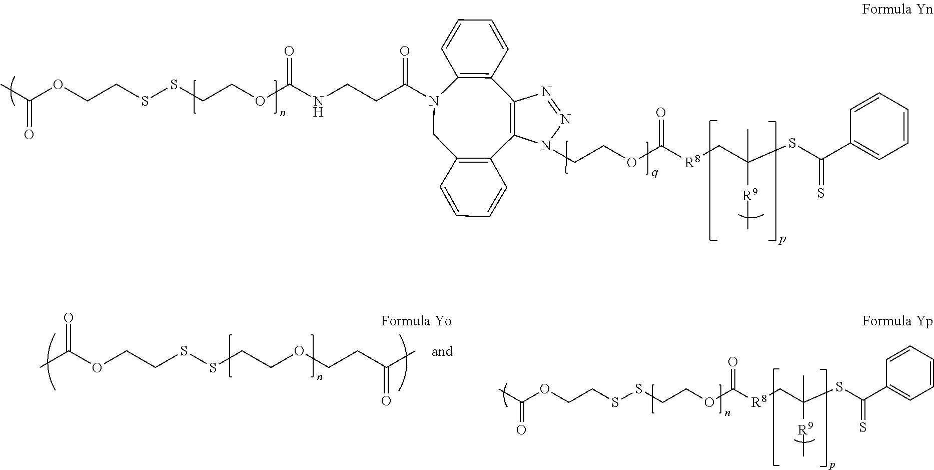

or has a portion represented by Formula Y′-CMP:

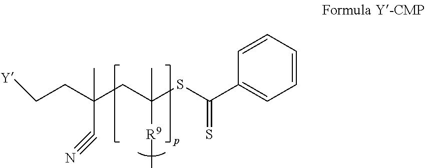

where: the left bracket “(” indicates the bond between X and Y; the right or bottom bracket and “)” indicates the bond between Y and Z; n is an integer from about 1 to 100; p is an integer from about 2 to 150; q is an integer from about 1 to 44; R⁸ is —CH₂— or —CH₂—CH₂—C(CH₃) (CN)—; R⁹ is a direct bond or —CH₂—CH₂—NH—C(O)—; and Y′ represents the remaining portion of Y.

In another aspect of the above, n is about 40 to 80, p is about 10 to 100, q is about 3 to 20, R⁸ is —CH₂—CH₂—C(CH₃)(CN)—; and when R⁹ is —CH₂—CH₂—NH—C(O)—, Z is galactose or N-acetylgalactosamine conjugated at its C1.

In still another aspect of the above, Y comprises Formula Ya, Formula Yb, Formula Yh, Formula Yi, Formula Yk, Formula Ym or Formula Yn, particularly Formula Ya, Formula Yb, Formula Ym or Formula Yn.

X can further comprise: a foreign transplant antigen against which transplant recipients develop an unwanted immune response; a foreign food, animal, plant or environmental antigen against which patients develop an unwanted immune response; a foreign therapeutic agent against which patients develop an unwanted immune response; or a synthetic self-antigen against the endogenous version of which patients develop an unwanted immune response, or a tolerogenic portion thereof.

The disclosure also pertains to a method of treatment for an unwanted immune response against an antigen by administering to a mammal in need of such treatment an effective amount of a composition comprising a compound of Formula 1 as discussed above. In such method the composition can be administered for clearance of a circulating protein or peptide or antibody that specifically binds to antigen moiety X, which circulating protein or peptide or antibody is causatively involved in transplant rejection, immune response against a therapeutic agent, autoimmune disease, hypersensitivity and/or allergy. The composition can be administered in an amount effective to reduce a concentration of the antibodies that are causatively involved in transplant rejection, immune response against a therapeutic agent, autoimmune disease, hypersensitivity and/or allergy in blood of the patient by at least 50% w/w, as measured at a time between about 12 to about 48 hours after the administration. The composition can administered for tolerization of a patient with respect to antigen moiety X.

Yet another aspect of the disclosure provides a composition comprising a compound of Formula 2:

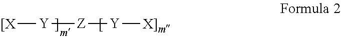

where: m′ is zero or an integer from about 1 to 10, m″ is zero or an integer from about 1 to 10, and the sum of m′+m″ is an integer from about 1 to 10 but is at least 1; each X is a foreign antigen or self-antigen against which a patient develops an unwanted immune response, or a tolerogenic portion thereof; each Y is a linker moiety or a direct bond, or an antibody, antibody fragment, peptide or other ligand that specifically binds X; and Z is a liver-targeting moiety, provided that X is not interferon, Ribavirin, Nexavar/Sorafenib, Erbitus/Cetuximab, Avastatin/bevacizumab or Herceptin/trastuzumab when m′+m″ equals 1 and Z is DOM 26h-196-61

In the above aspect involving Formula 2, Z can comprise an ASGPR-targeted antibody, an ASGPR-targeted antibody fragment, an ASGPR-targeted peptide, an ASGPR-targeted scFv, or another ASGPR ligand. Y can be a linker having an immunoproteosome cleavage site. X can comprise a group of related antigens against which a patient develops an unwanted immune response or a group of tolerogenic fragments thereof. For example, X can be selected from the groups comprising:
two or more of insulin, proinsulin, preproinsulin, glutamic acid decarboxylase-65 GAD-67, glucose-6 phosphatase 2, insulinoma-associated protein 2, insulinoma-associated protein 2β, ICA69, ICA12, carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5;
two or more of myelin basic protein, myelin oligodendrocyte glycoprotein, myelin proteolipid protein, SEQ ID NO:11, SEQ ID NO:12, SEQ ID NO:13, SEQ ID NO:14, SEQ ID NO:15, SEQ ID NO:16, and SEQ ID NO:17; and
two or more of SEQ ID NO:24, SEQ ID NO:25, SEQ ID NO:26, and SEQ ID NO:27.

The disclosure also pertains to a method of treatment for an unwanted immune response against an antigen by administering to a mammal in need of such treatment an effective amount of a composition comprising a compound of Formula 2 as discussed above. In such method the composition can be administered for clearance of a circulating protein or peptide or antibody that specifically binds to antigen moiety X, which circulating protein or peptide or antibody is causatively involved in transplant rejection, immune response against a therapeutic agent, autoimmune disease, hypersensitivity and/or allergy. The composition can be administered in an amount effective to reduce a concentration of the antibodies that are causatively involved in transplant rejection, immune response against a therapeutic agent, autoimmune disease, hypersensitivity and/or allergy in blood of the patient by at least 50% w/w, as measured at a time between about 12 to about 48 hours after the administration. The composition can administered for tolerization of a patient with respect to antigen moiety X.

### BRIEF DESCRIPTION OF THE DRAWINGS

FIG. 1A is a graph showing galactose conjugation (F1aA-PE-m₄-n₈₀ (Gal-PE)) preferentially targets OVA to sinusoidal endothelia cells of the liver (LSECs).

FIG. 1B is a graph showing galactose conjugation (F1aA-PE-m₄-n₈₀ (Gal-PE)) preferentially targets OVA to Kupffer cells of the liver (KC).

FIG. 1C is a graph showing galactose conjugation (F1aA-PE-m₄-n₈₀ (Gal-PE)) preferentially targets OVA to hepatocytes of the liver.

FIG. 1D is a graph showing galactose conjugation (F1aA-PE-m₄-n₈₀ (Gal-PE)) preferentially targets OVA to other antigen-presenting cells of the liver (APCs).

FIG. 2 is a graph showing proliferation of OTI CD8+ T cells in mice treated with F1aA-OVA-m₄-n₈₀ (Gal-OVA), OVA or saline (i.e. naïve).

FIG. 3A is a graph showing the percentage of OT-I CD8⁺ T cells presenting surface markers [[(A.)]] PD-1+ in generations of proliferating T cells treated with saline, OVA or F1aA-OVA-m₄-n₈₀ (GAL-OVA).

FIG. 3B is a graph showing the percentage of OT-I CD8⁺ T cells presenting surface markers Annexin-V+ in generations of proliferating T cells treated with saline, OVA or F1aA-OVA-m₄-n₈₀ (GAL-OVA).

FIG. 4 is a graph showing galactose conjugation (F1aA-OVA-m₄-n₈₀ (Gal-OVA)) decreases the immunogenicity of OVA as determined by OVA-specific antibody titers (shown in Ab titers log-1).

FIG. 5 shows that F1aA-OVA-m₄-n₈₀ (Gal-OVA) is able to deplete OVA-specific antibodies from the serum.

FIG. 6 shows that F1aA-OVA-m₄-n₈₀ (mGal-OVA), F1b-OVA-m₁-n₄₄-p₃₄ (pGal-OVA), and N′-DOM-Gly₃Ser-OVA-Gly₃Ser-6×His-C′ (Dom-OVA) are able to mitigate the OVA-specific immune response in draining lymphnodes after intradermal challenge with OVA and the adjuvant LPS.

FIG. 7A shows the characterization of F1aA-OVA-m₄-n₈₀ and F1b-OVA-m₁-n₄₄-p₃₄ by Size-exclusion HPCL traces of F1aA-OVA-m₄-n₈₀ (magenta), F1b-OVA-m₁-n₄₄-p₃₄ (blue) and unconjugated OVA (black). Shift to the left represents an increase in molecular weight.

FIG. 7B shows the characterization of F1aA-OVA-m₄-n₈₀ and F1b-OVA-m₁-n₄₄-p₃₄ by Polyacrylamide gel demonstrating increased molecular weight after OVA conjugation: (1.) Unconjugated OVA, (2.) F1aA-OVA-m₄-n₈₀ and (3.) F1b-OVA-m₁-n₄₄-p₃₄.

FIG. 8 is a graph showing the normalized quantity of N-OVA-Gly₃Ser-6×His-C(OVA), N-DOM-Gly₃Ser-OVA-Gly₃Ser-6×His-C (DOM-OVA), or N-OVA-Gly₃Ser-DOM-Gly₃Ser-6×His-C(OVA-DOM) in the circulation of mice after injection as an i.v. bolus.

FIG. 9A is a series of graphs showing the titer of anti-OVA IgG antibodies in the circulation of individual mice after treatment with saline, DOM and OVA, DOM-OVA, or OVA-DOM. Production of anti-OVA IgG was induced by i.v. injections of OVA alone. Treatment times are indicated by vertical dashed lines. Titer is calculated as log 10 of the maximal fold dilution of plasma with detectable anti-OVA IgG.

FIG. 9B is a series of graphs showing the titer of anti-OVA IgG antibodies in the circulation of individual mice after treatment with saline, DOM and OVA, DOM-OVA, or OVA-DOM. Production of anti-OVA IgG was induced by i.v. injections of OVA and CpG-B. Treatment times are indicated by vertical dashed lines. Titer is calculated as logo of the maximal fold dilution of plasma with detectable anti-OVA IgG.

### DETAILED DESCRIPTION

The two known asialoglycoprotein receptors (“ASGPRs”) are expressed on hepatocytes and liver sinusoidal endothelial cells (or “LSECs”). Other galactose/galactosamine/N-acetylgalactosamine receptors can be found in various forms on multiple cell types [e.g., dendritic cells, hepatocytes, LSECs, and Kupffer cells]. Dendritic cells are considered “professional antigen presenting cells,” because their primary function is to present antigens to the immune system for generating immune responses. Some cells within the liver are known to be able to present antigens, but the liver is more known to be involved in tolerogenesis. The liver is understood to be a tolerogenic organ. For example, lower incidences of rejection are reported in cases of multiple organ transplants when the liver is one of the organs transplanted. LSECs are much newer to the literature; consequently their role in tolerogenesis and/or moderation of inflammatory immune responses is not yet widely acknowledged or well understood. However, it is becoming clear that they also can play a significant role in the induction of antigen-specific tolerance.

One of the distinctive features of the erythrocyte surface is its glycosylation, i.e., the presence of significant numbers of glycosylated proteins. Indeed, the glycophorins (e.g., glycophorin A) have been employed as targets for erythrocyte binding. Glycophorins are proteins with many covalently attached sugar chains, the end terminus of which is sialic acid. As an erythrocyte ages and becomes ripe for clearance, the terminal sialic acid of its glycophorins tends to be lost, leaving N-acetylgalactosamine at the free end. N-acetylgalactosamine is a ligand selectively received by the ASGPR associated with hepatic cells, leading to binding of N-acetylgalactosamine-containing substances by hepatic cells and their subsequent uptake and processing in the liver.

Heretofore, it has been understood by those skilled in the art that glycosylation of a therapeutic agent in a manner that results in hepatic targeting should be avoided due to first-pass clearance by the liver resulting in poor circulation half-life of the therapeutic agent. By the same token, some monoclonal antibodies need to be specifically glycosylated at ASN297 for optimal binding to their Fc receptors. It has now surprisingly been found that galactosylation can be used in a manner that induces tolerogenesis.

The present disclosure provides certain therapeutic compositions that are targeted for delivery to (and for uptake by) the liver, particularly hepatocytes, LSECs, Kupffer cells and/or stellate cells, more particularly hepatocytes and/or LSECs, and even more particularly to specifically bind ASGPR. Liver-targeting facilitates two mechanisms of treatment: tolerization and clearance. Tolerization takes advantage of the liver's role in clearing apoptotic cells and processing their proteins to be recognized by the immune system as “self,” as well as the liver's role in sampling peripheral proteins for immune tolerance. Clearance takes advantage of the liver's role in blood purification by rapidly removing and breaking down toxins, polypeptides and the like. Targeting of these compositions to the liver is accomplished by a galactosylating moiety (e.g., galactose, galactosamine and N-acetylgalactosamine, particularly conjugated at C1, C2 or C6), by another liver-targeting moiety (e.g., a monoclonal antibody, or a fragment or a scFv thereof), or by de-sialylating a polypeptide for which such liver-targeting is desired. The galactosylating or other liver-targeting moiety can be chemically conjugated or recombinantly fused to an antigen, whereas desialylation exposes a galactose-like moiety on an antigen polypeptide. The antigen can be endogenous (a self-antigen) or exogenous (a foreign antigen), including but not limited to: a foreign transplant antigen against which transplant recipients develop an unwanted immune response (e.g., transplant rejection), a foreign food, animal, plant or environmental antigen to which patients develop an unwanted immune (e.g., allergic or hypersensitivity) response, a therapeutic agent to which patients develop an unwanted immune response (e.g., hypersensitivity and/or reduced therapeutic activity), a self-antigen to which patients develop an unwanted immune response (e.g., autoimmune disease), or a tolerogenic portion (e.g., a fragment or an epitope) thereof; these compositions are useful for inducing tolerization to the antigen. Alternatively, the galactosylating or other liver-targeting moiety can be conjugated to an antibody, antibody fragment or ligand that specifically binds a circulating protein or peptide or antibody, which circulating protein or peptide or antibody is causatively involved in transplant rejection, immune response against a therapeutic agent, autoimmune disease, and/or allergy (as discussed above); these compositions are useful for clearing the circulating protein, peptide or antibody. Accordingly, the compositions of the present disclosure can be used for treating an unwanted immune response, e.g., transplant rejection, an immune response against a therapeutic agent, an autoimmune disease, and/or an allergy. Also provided are pharmaceutical compositions containing a therapeutically effective amount of a composition of the disclosure admixed with at least one pharmaceutically acceptable excipient. In another aspect, the disclosure provides methods for the treatment of an unwanted immune response, such as transplant rejection, response against a therapeutic agent, autoimmune disease or allergy.

### Definitions

As used in the present specification, the following words and phrases are generally intended to have the meanings as set forth below, except to the extent that the context in which they are used indicates otherwise.

The singular forms “a,” “an,” and “the” include plural referents, unless the context clearly indicates otherwise.

The term “about” when used in connection with a numerical value is meant to encompass numerical values within a range typically having a lower limit that is, e.g., 5-10% smaller than the indicated numerical value and having an upper limit that is, e.g., 5-10% larger than the indicated numerical value.

An “antigen” is any substance that serves as a target for the receptors of an adaptive immune response, such as the T cell receptor, B cell receptor or an antibody. An antigen may originate from within the body (“self,” “auto” or “endogenous”). An antigen may originate from outside the body (“non-self,” “foreign” or “exogenous”), having entered, for example, by inhalation, ingestion, injection, or transplantation. Foreign antigens include, but are not limited to, food antigens, animal antigens, plant antigens, environmental antigens, therapeutic agents, as well as antigens present in an allograft transplant.

An “antigen-binding molecule” as used herein relates to molecules, in particular to proteins such as immunoglobulin molecules, which contain antibody variable regions providing a specific binding to an epitope. The antibody variable region can be present in, for example, a complete antibody, an antibody fragment, and a recombinant derivative of an antibody or antibody fragment. The term “antigen-binding fragment” of an antibody (or “binding portion”), as used herein, refers to one or more fragments of an antibody that retain the ability to specifically bind a target sequence. Antigen-binding fragments containing antibody variable regions include (without limitation) “Fv”, “Fab”, and “F(ab′) 2” regions, “single domain antibodies (sdAb)”, “nanobodies”, “single chain Fv (scFv)” fragments, “tandem scFvs” (V_{H}A-V_{L}A-V_{H}B-V_{L}B), “diabodies”, “triabodies” or “tribodies”, “single-chain diabodies (scDb)”, and “bi-specific T-cell engagers (BiTEs)”.

A “chemical modification” refers to a change in the naturally-occurring chemical structure of one or more amino acids of a polypeptide. Such modifications can be made to a side chain or a terminus, e.g., changing the amino-terminus or carboxyl terminus. In some embodiments, the modifications are useful for creating chemical groups that can conveniently be used to link the polypeptides to other materials, or to attach a therapeutic agent.

The term “comprising”, which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics.

“Conservative changes” can generally be made to an amino acid sequence without altering activity. These changes are termed “conservative substitutions” or mutations; that is, an amino acid belonging to a grouping of amino acids having a particular size or characteristic can be substituted for another amino acid. Substitutes for an amino acid sequence can be selected from other members of the class to which the amino acid belongs. For example, the nonpolar (hydrophobic) amino acids include alanine, leucine, isoleucine, valine, proline, phenylalanine, tryptophan, methionine, and tyrosine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Such substitutions are not expected to substantially affect apparent molecular weight as determined by polyacrylamide gel electrophoresis or isoelectric point. Conservative substitutions also include substituting optical isomers of the sequences for other optical isomers, specifically D amino acids for L amino acids for one or more residues of a sequence. Moreover, all of the amino acids in a sequence can undergo a D to L isomer substitution. Exemplary conservative substitutions include, but are not limited to, Lys for Arg and vice versa to maintain a positive charge; Glu for Asp and vice versa to maintain a negative charge; Ser for Thr so that a free-OH is maintained; and Gln for Asn to maintain a free —NH₂. Yet another type of conservative substitution constitutes the case where amino acids with desired chemical reactivities are introduced to impart reactive sites for chemical conjugation reactions, if the need for chemical derivativization arises. Such amino acids include but are not limited to Cys (to insert a sulfhydryl group), Lys (to insert a primary amine), Asp and Glu (to insert a carboxylic acid group), or specialized noncanonical amino acids containing ketone, azide, alkyne, alkene, and tetrazine side-chains. Conservative substitutions or additions of free —NH₂ or —SH bearing amino acids can be particularly advantageous for chemical conjugation with the linkers and galactosylating moieties of Formula 1. Moreover, point mutations, deletions, and insertions of the polypeptide sequences or corresponding nucleic acid sequences can in some cases be made without a loss of function of the polypeptide or nucleic acid fragment. Substitutions can include, e.g., 1, 2, 3, 4, 5, 6, 7, 8, 9, 10, 15, 20, 25, 30, 35, 40, 45, 50 or more residues. A variant usable in the present invention may exhibit a total number of up to 200 (up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200) changes in the amino acid sequence (i.e. exchanges, insertions, deletions, N-terminal truncations, and/or C-terminal truncations). The amino acid residues described herein employ either the single letter amino acid designator or the three-letter abbreviation in keeping with the standard polypeptide nomenclature, J. Biol. Chem., (1969), 243, 3552-3559. All amino acid residue sequences are represented herein by formulae with left and right orientation in the conventional direction of amino-terminus to carboxy-terminus.

The terms “effective amount” or “therapeutically effective amount” refer to that amount of a composition of the disclosure that is sufficient to effect treatment, as defined below, when administered to a mammal in need of such treatment. This amount will vary depending upon the subject and disease condition being treated, the weight and age of the subject, the severity of the disease condition, the particular composition of the disclosure chosen, the dosing regimen to be followed, timing of administration, manner of administration and the like, all of which can readily be determined by one of ordinary skill in the art.

An “epitope”, also known as antigenic determinant, is the segment of a macromolecule, e.g. a protein, which is recognized by the adaptive immune system, such as by antibodies, B cells, or T cells. An epitope is that part or segment of a macromolecule capable of binding to an antibody or antigen-binding fragment thereof. In this context, the term “binding” in particular relates to a specific binding. In the context of the present invention it is preferred that the term “epitope” refers to the segment of protein or polyprotein that is recognized by the immune system.

The term galactose is well known in the art and refers to a monosaccharide sugar that exists both in open-chain form and in cyclic form, having D- and L-isomers. In the cyclic form there are two anomers, namely alpha and beta. In the alpha form, the C1 alcohol group is in the axial position, whereas in the beta form, the C1 alcohol group is in the equatorial position. In particular, “galactose” refers to the cyclic six-membered pyranose, more in particular the D-isomer and even more particularly the alpha-D-form (α-D-galactopyranose). The structure and numbering of galactose is illustrated below.

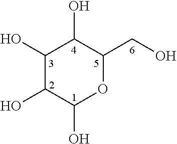 [SMILES: OCC1OC(O)C(O)C(O)C1O]

The term “galactosylating moiety” refers to a particular type of liver-targeting moiety. Galactosylating moieties include, but are not limited to a galactose, galactosamine and/or N-acetylgalactosamine residue.

The term “liver-targeting moiety”, refers to moieties having the ability to direct, e.g., a polypeptide, to the liver. The liver comprises different cell types, including but not limited to hepatocytes, sinusoidal epithelial cells, Kupffer cells, stellate cells, and/or dendritic cells. Typically, a liver-targeting moiety directs a polypeptide to one or more of these cells. On the surface of the respective liver cells, receptors are present which recognize and specifically bind the liver-targeting moiety. Liver-targeting can be achieved by chemical conjugation of an antigen or ligand to a galactosylating moiety, desialylation of an antigen or ligand to expose underlying galactosyl moieties, recombinant fusion or chemical conjugation of an antigen or ligand to an ASGPR-binding moiety, or specific binding of an endogenous antibody to an antigen or ligand, where the antigen or ligand is: desialylated to expose underlying galactosyl moieties, conjugated to a galactosylating moiety, or recombinantly fused or chemically conjugated to an ASGPR-binding moiety. Naturally occurring desialylated proteins are not encompassed within the scope of the present disclosure.

The “numerical values” and “ranges” provided for the various substituents are intended to encompass all integers within the recited range. For example, when defining n as an integer representing a mixture including from about 1 to 100, particularly about 8 to 90 and more particularly about 40 to 80 ethylene glycol groups, where the mixture typically encompasses the integer specified as n±about 10% (or for smaller integers from 1 to about 25, ±3), it should be understood that n can be an integer from about 1 to 100 (e.g., 1, 2, 3, 4, 5, 6, 7, 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95, 99, 100, 105 or 110) and that the disclosed mixture encompasses ranges such as 1-4, 2-4, 2-6, 3-8, 7-13, 6-14, 18-23, 26-30, 42-50, 46-57, 60-78, 85-90, 90-110 and 107-113 ethylene glycol groups. The combined terms “about” and “±10%” or “±3” should be understood to disclose and provide specific support for equivalent ranges wherever used.

The term “optional” or “optionally” means that the subsequently described event or circumstance may or may not occur, and that the description includes instances where said event or circumstance occurs and instances in which it does not.

A peptide that specifically binds a particular target is referred to as a “ligand” for that target.

A “polypeptide” is a term that refers to a chain of amino acid residues, regardless of post-translational modification (e.g., phosphorylation or glycosylation) and/or complexation with additional polypeptides, and/or synthesis into multisubunit complexes with nucleic acids and/or carbohydrates, or other molecules. Proteoglycans therefore also are referred to herein as polypeptides. A long polypeptide (having over about 50 amino acids) is referred to as a “protein.” A short polypeptide (having fewer than about 50 amino acids) is referred to as a “peptide.” Depending upon size, amino acid composition and three dimensional structure, certain polypeptides can be referred to as an “antigen-binding molecule,” “antibody,” an “antibody fragment” or a “ligand.” Polypeptides can be produced by a number of methods, many of which are well known in the art. For example, polypeptides can be obtained by extraction (e.g., from isolated cells), by expression of a recombinant nucleic acid encoding the polypeptide, or by chemical synthesis. Polypeptides can be produced by, for example, recombinant technology, and expression vectors encoding the polypeptide introduced into host cells (e.g., by transformation or transfection) for expression of the encoded polypeptide

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “purified” as used herein with reference to a polypeptide refers to a polypeptide that has been chemically synthesized and is thus substantially uncontaminated by other polypeptides, or has been separated or isolated from most other cellular components by which it is naturally accompanied (e.g., other cellular proteins, polynucleotides, or cellular components). An example of a purified polypeptide is one that is at least 70%, by dry weight, free from the proteins and naturally occurring organic molecules with which it naturally associates. A preparation of a purified polypeptide therefore can be, for example, at least 80%, at least 90%, or at least 99%, by dry weight, the polypeptide. Polypeptides also can be engineered to contain a tag sequence (e.g., a polyhistidine tag, a myc tag, a FLAG® tag, or other affinity tag) that facilitates purification or marking (e.g., capture onto an affinity matrix, visualization under a microscope). Thus a purified composition that comprises a polypeptide refers to a purified polypeptide unless otherwise indicated. The term “isolated” indicates that the polypeptides or nucleic acids of the disclosure are not in their natural environment. Isolated products of the disclosure can thus be contained in a culture supernatant, partially enriched, produced from heterologous sources, cloned in a vector or formulated with a vehicle, etc.

The term “sequence identity” is used with regard to polypeptide sequence comparisons. This expression in particular refers to a percentage of sequence identity, for example at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide. Particularly, the polypeptide in question and the reference polypeptide exhibit the indicated sequence identity over a continuous stretch of 20, 30, 40, 45, 50, 60, 70, 80, 90, 100 or more amino acids or over the entire length of the reference polypeptide.

“Specific binding,” as that term is commonly used in the biological arts, refers to a molecule that binds to a target with a relatively high affinity as compared to non-target tissues, and generally involves a plurality of non-covalent interactions, such as electrostatic interactions, van der Waals interactions, hydrogen bonding, and the like. Specific binding interactions characterize antibody-antigen binding, enzyme-substrate binding, and certain protein-receptor interactions; while such molecules might bind tissues besides their specific targets from time to time, to the extent that such non-target binding is inconsequential, the high-affinity binding pair can still fall within the definition of specific binding.

The term “treatment” or “treating” means any treatment of a disease or disorder in a mammal, including:
preventing or protecting against the disease or disorder, that is, causing the clinical symptoms not to develop;
inhibiting the disease or disorder, that is, arresting or suppressing the development of clinical symptoms; and/or
relieving the disease or disorder, that is, causing the regression of clinical symptoms.

The term “unwanted immune response” refers to a reaction by the immune system of a subject, which in the given situation is not desirable. The reaction of the immune system is unwanted if such reaction does not lead to the prevention, reduction, or healing of a disease or disorder but instead causes, enhances or worsens a disorder or disease. Typically, a reaction of the immune system causes, enhances or worsens a disease if it is directed against an inappropriate target. Exemplified, an unwanted immune response includes but is not limited to transplant rejection, immune response against a therapeutic agent, autoimmune disease, and allergy or hypersensitivity.

The term “variant” is to be understood as a protein which differs in comparison to the protein from which it is derived by one or more changes in its length, sequence, or structure. The polypeptide from which a protein variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence or structure in comparison to the parent molecule. Also encompassed modified molecules such as but not limited to post-translationally modified proteins (e.g. glycosylated, phosphorylated, ubiquitinated, palmitoylated, or proteolytically cleaved proteins) and modified nucleic acids such as methylated DNA. Also mixtures of different molecules such as but not limited to RNA-DNA hybrids, are encompassed by the term “variant”. Naturally occurring and artificially constructed variants are to be understood to be encompassed by the term “variant” as used herein. Further, the variants usable in the present invention may also be derived from homologs, orthologs, or paralogs of the parent molecule or from artificially constructed variant, provided that the variant exhibits at least one biological activity of the parent molecule, i.e. is functionally active. A variant can be characterized by a certain degree of sequence identity to the parent polypeptide from which it is derived. More precisely, a protein variant in the context of the present disclosure may exhibit at least 80% sequence identity to its parent polypeptide. Preferably, the sequence identity of protein variants is over a continuous stretch of 20, 30, 40, 45, 50, 60, 70, 80, 90, 100 or more amino acids.

### Compositions

One aspect of the present disclosure relates to compositions, pharmaceutical formulations, and methods of treatment employing such compositions, as represented by Formula 1:

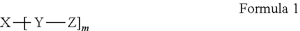

where:
m is an integer from about 1 to 100, particularly from about 1 to 20, and most particularly 1 to about 10;
X is an antigen moiety, particularly a foreign antigen or self-antigen against which a patient develops an unwanted immune response, or a tolerogenic portion (e.g., a fragment or an epitope) of such an antigen moiety;
Y is a linker moiety or a direct bond, or an antibody, antibody fragment, peptide or other ligand that specifically binds X; and
Z is a liver-targeting moiety, in particular galactosylating moiety.

The value for m in Formula 1 will depend upon the nature of X, in that each antigen, antibody, antibody fragment or ligand will have an individual number and density of sites (predominantly the N-terminal amine, lysine residues and cysteine residues) to which a linker or galactosylating moiety can be bound. Antigens having a limited number of such sites can be derivatized, for example, at the N or C terminus, by adding lysine or cysteine residues (optionally via a cleavable linker, particularly a linker having an immunoproteosome cleavage site). Generally, it is preferred to provide an adequate degree of galactosylation in compositions of Formula 1 so as to facilitate uptake by liver cells. Pharmaceutical formulations and methods of the disclosure can employ a cocktail of compositions of Formula 1, respectively bearing different X moieties (e.g., several epitopes associated with a particular unwanted immune response).

The compositions of Formula 1 include the sub-genuses where X is a foreign transplant antigen against which transplant recipients develop an unwanted immune response (e.g., transplant rejection), a foreign food, animal, plant or environmental antigen against which patients develop an unwanted immune (e.g., allergic or hypersensitivity) response, a foreign therapeutic agent against which patients develop an unwanted immune response (e.g., hypersensitivity and/or reduced therapeutic activity), or a self-antigen against which patients develop an unwanted immune response (e.g., autoimmune disease); where Y is a linker of Formulae Ya through Yp; and/or where Z is galactose, galactosamine or N-acetylgalactosamine, as illustrated by Formulae 1a through 1p as described below with reference to the Reaction Schemes.

The disclosure further provides a pharmaceutically acceptable composition represented by Formula 2:

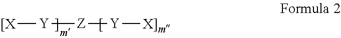

where:
m′ is zero or an integer from about 1 to 10, m″ is zero or an integer from about 1 to 10, and the sum of m′+m″ is an integer from about 1 to 10 but is at least 1;
X is an antigen moiety, particularly a foreign antigen or self-antigen against which a patient develops an unwanted immune response, or a tolerogenic portion (e.g., a fragment or an epitope) of such an antigen moiety;
Y is a linker moiety or a direct bond, or an antibody, antibody fragment, peptide or other ligand that specifically binds X; and
Z is a liver-targeting moiety, in particular an ASGPR-targeted antibody, an ASGPR-targeted antibody fragment, an ASGPR-targeted peptide, an ASGPR-targeted scFv, or another ASGPR ligand,
   such composition optionally including amino acid sequences to facilitate isolation and purification [e.g., a “His tag” or “6×His” having the sequence: HHHHHH (SEQ ID NO:1) and additional linkers [e.g., “Gly₃Ser” having the sequence: GGGS (SEQ ID NO:2)]. In the compositions of Formula 2 where m′+m″ is greater than one, the X moieties can be the same or different, and the Y moieties can be the same or different. The value for m′+m″ will be smaller (e.g., 3 or less) when X is a full protein, or larger (up to about 10) when X is a peptide. The linker(s) “Y” can advantageously comprise a cleavage site, particularly an immunoproteosome cleavage site.

The compositions of Formula 2 include the sub-genuses where X is a foreign transplant antigen against which transplant recipients develop an unwanted immune response (e.g., transplant rejection), a foreign food, animal, plant or environmental antigen against which patients develop an unwanted immune (e.g., allergic or hypersensitivity) response, a foreign therapeutic agent against which patients develop an unwanted immune response (e.g., hypersensitivity and/or reduced therapeutic activity), or a self-antigen against which patients develop an unwanted immune response (e.g., autoimmune disease).

Alternatively, in the compositions of Formula 1 and/or Formula 2, X can be an antibody, antibody fragment or ligand that specifically binds a circulating protein or peptide or antibody, which circulating protein or peptide or antibody is causatively involved in transplant rejection, immune response against a therapeutic agent, autoimmune disease, hypersensitivity and/or allergy.

The compositions of Formula 2 can be prepared as fusion proteins and can include several components useful in their preparation and purification, such as a signal or leader sequence that directs the expressed polypeptide to a specific transport pathway and is subsequently cleaved from the polypeptide; therefore the signal sequence can be part of the expressed protein and DNA encoding it, but not part of the final composition. One example of a mammalian signal sequence used in expression systems is the Ig κ-chain sequence, which directs protein secretion: METDTLLLWVLLLWVPGSTG (SEQ ID NO: 3). Similarly, one example of a common bacterial signal sequence used in expression systems is the pelB signal sequence, which directs expressed protein into the bacterial periplasm:

(SEQ ID NO: 4)
MKYLLPTAAAGLLLLAAQPAMA.

### Antigens

The antigen employed as X in the compositions of Formula 1 and/or Formula 2 can be a protein or a peptide, e.g. the antigen may be a complete or partial therapeutic agent, a full-length transplant protein or peptide thereof, a full-length autoantigen or peptide thereof, a full-length allergen or peptide thereof, and/or a nucleic acid, or a mimetic of an aforementioned antigen.

Antigens employed in the practice of the present disclosure can be one or more of the following:
Therapeutic agents that are proteins, peptides, antibodies and antibody-like molecules, including antibody fragments and fusion proteins with antibodies and antibody fragments. These include human, non-human (such as mouse) and non-natural (i.e., engineered) proteins, antibodies, chimeric antibodies, humanized antibodies, and non-antibody binding scaffolds, such as fibronectins, DARPins, knottins, and the like.
Human allograft transplantation antigens against which transplant recipients develop an unwanted immune response.
Self-antigens that cause an unwanted, autoimmune response. Those skilled in the art will appreciate that while self-antigens are of an endogenous origin in an autoimmune disease patient, the polypeptides employed in the disclosed compositions are typically synthesized exogenously (as opposed to being purified and concentrated from a source of origin).
Foreign antigens, such as food, animal, plant and environmental antigens, against which a patient experiences an unwanted immune response. Those skilled in the art will appreciate that while a therapeutic protein can also be considered a foreign antigen due to its exogenous origin, for purposes of clarity in the description of the present disclosure such therapeutics are described as a separate group. Similarly, a plant or an animal antigen can be eaten and considered a food antigen, and an environmental antigen may originate from a plant. They are, however, all foreign antigens. In the interest of simplicity no attempt will be made to describe distinguish and define all of such potentially overlapping groups, as those skilled in the art can appreciate the antigens that can be employed in the compositions of the disclosure, particularly in light of the detailed description and examples.
   The antigen can be a complete protein, a portion of a complete protein, a peptide, or the like, and can be derivatized (as discussed above) for attachment to a linker and/or galactosylating moiety, can be a variant and/or can contain conservative substitutions, particularly maintaining sequence identity, and/or can be desialylated.

In the embodiments where the antigen is a therapeutic protein, peptide, antibody or antibody-like molecule, specific antigens can be selected from: Abatacept, Abciximab, Adalimumab, Adenosine deaminase, Ado-trastuzumab emtansine, Agalsidase alfa, Agalsidase beta, Aldeslukin, Alglucerase, Alglucosidase alfa, α-1-proteinase inhibitor, Anakinra, Anistreplase (anisoylated plasminogen streptokinase activator complex), Antithrombin III, Antithymocyte globulin, Ateplase, Bevacizumab, Bivalirudin, Botulinum toxin type A, Botulinum toxin type B, C1-esterase inhibitor, Canakinumab, Carboxypeptidase G2 (Glucarpidase and Voraxaze), Certolizumab pegol, Cetuximab, Collagenase, Crotalidae immune Fab, Darbepoetin-α, Denosumab, Digoxin immune Fab, Dornase alfa, Eculizumab, Etanercept, Factor Vila, Factor VIII, Factor IX, Factor XI, Factor XIII, Fibrinogen, Filgrastim, Galsulfase, Golimumab, Histrelin acetate, Hyaluronidase, Idursulphase, Imiglucerase, Infliximab, Insulin [including recombinant human insulin (“rHu insulin”) and bovine insulin], Interferon-α2a, Interferon-α2b, Interferon-β1a, Interferon-β1b, Interferon-γ1b, Ipilimumab, L-arginase, L-asparaginase, L-methionase, Lactase, Laronidase, Lepirudin/hirudin, Mecasermin, Mecasermin rinfabate, Methoxy Natalizumab, Octreotide, Ofatumumab, Oprelvekin, Pancreatic amylase, Pancreatic lipase, Papain, Peg-asparaginase, Peg-doxorubicin HCl, PEG-epoetin-β, Pegfilgrastim, Peg-Interferon-α2a, Peg-Interferon-α2b, Pegloticase, Pegvisomant, Phenylalanine ammonia-lyase (PAL), Protein C, Rasburicase (uricase), Sacrosidase, Salmon calcitonin, Sargramostim, Streptokinase, Tenecteplase, Teriparatide, Tocilizumab (atlizumab), Trastuzumab, Type 1 alpha-interferon, Ustekinumab, vW factor. The therapeutic protein can be obtained from natural sources (e.g., concentrated and purified) or synthesized, e.g., recombinantly, and includes antibody therapeutics that are typically IgG monoclonal or fragments or fusions.

Particular therapeutic protein, peptide, antibody or antibody-like molecules include Abciximab, Adalimumab, Agalsidase alfa, Agalsidase beta, Aldeslukin, Alglucosidase alfa, Factor VIII, Factor IX, Infliximab, Insulin (including rHu Insulin), L-asparaginase, Laronidase, Natalizumab, Octreotide, Phenylalanine ammonia-lyase (PAL), or Rasburicase (uricase) and generally IgG monoclonal antibodies in their varying formats.

Another particular group includes the hemostatic agents (Factor VIII and IX), Insulin (including rHu Insulin), and the non-human therapeutics uricase, PAL and asparaginase.

Unwanted immune response in hematology and transplant includes autoimmune aplastic anemia, transplant rejection (generally), and Graft vs. Host Disease (bone marrow transplant rejection). In the embodiments where the antigen is a human allograft transplantation antigen, specific sequences can be selected from: subunits of the various MHC class I and MHC class II haplotype proteins (for example, donor/recipient differences identified in tissue cross-matching), and single-amino-acid polymorphisms on minor blood group antigens including RhCE, Kell, Kidd, Duffy and Ss. Such compositions can be prepared individually for a given donor/recipient pair.

In the embodiments where the antigen is a self-antigen, specific antigens (and the autoimmune disease with which they are associated) can be selected from:
In type 1 diabetes mellitus, several main antigens have been identified: insulin, proinsulin, preproinsulin, glutamic acid decarboxylase-65 (GAD-65 or glutamate decarboxylase 2), GAD-67, glucose-6 phosphatase 2 (IGRP or islet-specific glucose 6 phosphatase catalytic subunit related protein), insulinoma-associated protein 2 (IA-2), and insulinoma-associated protein 2 (IA-2B); other antigens include ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S100B, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. It should be noted that insulin is an example of an antigen that can be characterized both as a self-antigen and a therapeutic protein antigen. For example, rHu Insulin and bovine insulin are therapeutic protein antigens (that are the subject of unwanted immune attack), whereas endogenous human insulin is a self-antigen (that is the subject of an unwanted immune attack). Because endogenous human insulin is not available to be employed in a pharmaceutical composition a recombinant form is employed in the compositions of the disclosure.
   Human insulin, including an exogenously obtained form useful in the compositions of the disclosure, has the following sequence (UNIPROT P01308):

(SEQ ID NO: 5)
MALWMRLLPL LALLALWGPD PAAAFVNQHL CGSHLVEALY
LVCGERGFFY TPKTRREAED LQVGQVELGG GPGAGSLQPL
ALEGSLQKRG IVEQCCTSIC SLYQLENYCN.

GAD-65, including an exogenously obtained form useful in the compositions of the disclosure, has the following sequence (UNIPROT Q05329):

(SEQ ID NO: 6)
MASPGSGFWS FGSEDGSGDS ENPGTARAWC QVAQKFTGGI
GNKLCALLYG DAEKPAESGG SQPPRAAARK AACACDQKPC
SCSKVDVNYA FLHATDLLPA CDGERPTLAF LQDVMNILLQ
YVVKSFDRST KVIDFHYPNE LLQEYNWELA DQPQNLEEIL
MHCQTTLKYA IKTGHPRYFN QLSTGLDMVG LAADWLTSTA
NTNMFTYEIA PVFVLLEYVT LKKMREIIGW PGGSGDGIFS
PGGAISNMYA MMIARFKMFP EVKEKGMAAL PRLIAFTSEH
SHFSLKKGAA ALGIGTDSVI LIKCDERGKM IPSDLERRIL
EAKQKGFVPF LVSATAGTTV YGAFDPLLAV ADICKKYKIW
MHVDAAWGGG LLMSRKHKWK LSGVERANSV TWNPHKMMGV
PLQCSALLVR EEGLMQNCNQ MHASYLFQQD KHYDLSYDTG
DKALQCGRHV DVFKLWLMWR AKGTTGFEAH VDKCLELAEY
LYNIIKNREG YEMVFDGKPQ HTNVCFWYIP PSLRTLEDNE
ERMSRLSKVA PVIKARMMEY GTTMVSYQPL GDKVNFFRMV
ISNPAATHQD IDFLIEEIER LGQDL.

IGRP, including an exogenously obtained form useful in the compositions of the disclosure, has the following sequence (UNIPROT QN9QR⁹):

(SEQ ID NO: 7)
MDFLHRNGVLIIQHLQKDYRAYYTFLNFMSNVGDPRNIFFIYFPLCFQFN
QTVGTKMIWVAVIGDWLNLIFKWILFGHRPYWWVQETQIYPNHSSPCLEQ
FPTTCETGPGSPSGHAMGASCVWYVMVTAALSHTVCGMDKFSITLHRLTW
SFLWSVFWLIQISVCISRVFIATHFPHQVILGVIGGMLVAEAFEHTPGIQ
TASLGTYLKTNLFLFLFAVGFYLLLRVLNIDLLWSVPIAKKWCANPDWIH
IDTTPFAGLVRNLGVLFGLGFAINSEMFLLSCRGGNNYTLSFRLLCALTS
LTILQLYHFLQIPTHEEHLFYVLSFCKSASIPLTVVAFIPYSVHMLMKQS
GKKSQ.

In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor.
In Addison's Disease, main antigens include 21-hydroxylase, 17α-hydroxylase, and P450 side chain cleavage enzyme (P450scc); other antigens include ACTH receptor, P450c21 and P450c17.
In premature ovarian failure, main antigens include FSH receptor and α-enolase.
In autoimmune hypophysitis, or pituitary autoimmune disease, main antigens include pituitary gland-specific protein factor (PGSF) 1a and 2; another antigen is type 2 iodothyronine deiodinase.
In multiple sclerosis, main antigens include myelin basic protein (“MBP”), myelin oligodendrocyte glycoprotein (“MOG”) and myelin proteolipid protein (“PLP”).
   MBP, including an exogenously obtained form useful in the compositions of the disclosure, has the following sequence (UNIPROT P02686):

(SEQ ID NO: 8)
MGNHAGKRELNAEKASTNSETNRGESEKKRNLGELSRTTSEDNEVFGEAD
ANQNNGTSSQDTAVTDSKRTADPKNAWQDAHPADPGSRPHLIRLFSRDAP
GREDNTFKDRPSESDELQTIQEDSAATSESLDVMASQKRPSQRHGSKYLA
TASTMDHARHGFLPRHRDTGILDSIGRFFGGDRGAPKRGSGKDSHHPART
AHYGSLPQKSHGRTQDENPVVHFFKNIVTPRTPPPSQGKGRGLSLSRFSW
GAEGQRPGFGYGGRASDYKSAHKGFKGVDAQGTLSKIFKLGGRDSRSGSP
MARR.

MOG, including an exogenously obtained form useful in the compositions of the disclosure, has the following sequence (UNIPROT Q16653):

(SEQ ID NO: 9)
MASLSRPSLPSCLCSFLLLLLLQVSSSYAGQFRVIGPRHPIRALVGDEVE
LPCRISPGKNATGMEVGWYRPPFSRVVHLYRNGKDQDGDQAPEYRGRTEL
LKDAIGEGKVTLRIRNVRFSDEGGFTCFFRDHSYQEEAAMELKVEDPFYW
VSPGVLVLLAVLPVLLLQITVGLIFLCLQYRLRGKLRAEIENLHRTFDPH
FLRVPCWKITLFVIVPVLGPLVALIICYNWLHRRLAGQFLEELRNPF.

PLP, including an exogenously obtained form useful in the compositions of the disclosure, has the following sequence (UNIPROT P60201):

(SEQ ID NO: 10)
MGLLECCARCLVGAPFASLVATGLCFFGVALFCGCGHEALTGTEKLIETY
FSKNYQDYEYLINVIHAFQYVIYGTASFFFLYGALLLAEGFYTTGAVRQI
FGDYKTTICGKGLSATVTGGQKGRGSRGQHQAHSLERVCHCLGKWLGHPD
KFVGITYALTVVWLLVFACSAVPVYIYFNTWTTCQSIAFPSKTSASIGSL
CADARMYGVLPWNAFPGKVCGSNLLSICKTAEFQMTFHLFIAAFVGAAAT
LVSLLTFMIAATYNFAVLKLMGRGTKF.

Peptides/epitopes useful in the compositions of the disclosure for treating multiple sclerosis include some or all of the following sequences, individually in a composition of Formula 1, together in a cocktail of compositions of Formula 1, or fused in one or more compositions of Formula 2:

MBP13-32:
(SEQ ID NO: 11)
KYLATASTMDHARHGFLPRH;
MBP83-99:
(SEQ ID NO: 12)
ENPWHFFKNIVTPRTP;
MBP111-129:
(SEQ ID NO: 13)
LSRFSWGAEGQRPGFGYGG;
MBP146-170:
(SEQ ID NO: 14)
AQGTLSKIFKLGGRDSRSGSPMARR;
MOG1-20:
(SEQ ID NO: 15)
GQFRVIGPRHPIRALVGDEV;
MOG35-55:
(SEQ ID NO: 16)
MEVGWYRPPFSRWHLYRNGK;
and
PLP139-154:
(SEQ ID NO: 17)
HCLGKWLGHPDKFVGI.

In rheumatoid arthritis, main antigens include collagen II, immunoglobulin binding protein, the fragment crystallizable region of immunoglobulin G, double-stranded DNA, and the natural and cirtullinated forms of proteins implicated in rheumatoid arthritis pathology, including fibrin/fibrinogen, vimentin, collagen I and II, and alpha-enolase.
In autoimmune gastritis, a main antigen is H+, K+-ATPase.
In pernicious angemis, a main antigen is intrinsic factor.
In celiac disease, main antigens are tissue transglutaminase and the natural and deamidated forms of gluten or gluten-like proteins, such as alpha-, gamma-, and omega-gliadin, glutenin, hordein, secalin, and avenin. Those skilled in the art will appreciate, for example, that while the main antigen of celiac disease is alpha gliadin, alpha gliadin turns more immunogenic in the body through deamidation by tissue glutaminase converting alpha gliadin's glutamines to glutamic acid. Thus, while alpha gliadin is originally a foreign food antigen, once it has been modified in the body to become more immunogenic it can be characterized as a self-antigen.
In vitiligo, a main antigen is tyrosinase, and tyrosinase related protein 1 and 2.
   MART1, Melanoma antigen recognized by T cells 1, Melan-A, including an exogenously obtained form useful in the compositions of the disclosure, has the following sequence (UNIPROT Q16655):

(SEQ ID NO: 18)
MPREDAHFIYGYPKKGHGHSYTTAEEAAGIGILTVILGVLLLIGCWYCRR
RNGYRALMDKSLHVGTQCALTRRCPQEGFDHRDSKVSLQEKNCEPVVPNA
PPAYEKLSAEQSPPPYSP.

Tyrosinase, including an exogenously obtained form useful in the compositions of the disclosure, has the following sequence (UNIPROT P14679):

(SEQ ID NO: 19)
MLLAVLYCLLWSFQTSAGHFPRACVSSKNLMEKECCPPWSGDRSPCGQLSG
RGSCQNILLSNAPLGPQFPFTGVDDRESWPSVFYNRTCQCSGNFMGFNCGN
CKFGFWGPNCTERRLLVRRNIFDLSAPEKDKFFAYLTLAKHTISSDYVIPI
GTYGQMKNGSTPMFNDINIYDLFVWMHYYVSMDALLGGSEIWRDIDFAHEA
PAFLPWHRLFLLRWEQEIQKLTGDENFTIPYWDWRDAEKCDICTDEYMGGQ
HPTNPNLLSPASFFSSWQIVCSRLEEYNSHQSLCNGTPEGPLRRNPGNHDK
SRTPRLPSSADVEFCLSLTQYESGSMDKAANFSFRNTLEGFASPLTGIADA
SQSSMHNALHIYMNGTMSQVQGSANDPIFLLHHAFVDSIFEQWLRRHRPLQ
EVYPEANAPIGHNRESYMVPFIPLYRNGDFFISSKDLGYDYSYLQDSDPDS
FQDYIKSYLEQASRIWSWLLGAAMVGAVLTALLAGLVSLLCRHKRKQLPEE
KQPLLMEKEDYHSLYQSHL.

Melanocyte protein PMEL, gp100, including an exogenously obtained form useful in the compositions of the disclosure, has the following sequence (UNIPROT P40967):

(SEQ ID NO: 20)
MDLVLKRCLLHLAVIGALLAVGATKVPRNQDWLGVSRQLRTKAWNRQLYPE
WTEAQRLDCWRGGQVSLKVSNDGPTLIGANASFSIALNFPGSQKVLPDGQV
IWVNNTIINGSQVWGGQPVYPQETDDACIFPDGGPCPSGSWSQKRSFVYVW
KTWGQYWQVLGGPVSGLSIGTGRAMLGTHTMEVTVYHRRGSRSYVPLAHSS
SAFTITDQVPFSVSVSQLRALDGGNKHFLRNQPLTFALQLHDPSGYLAEAD
LSYTWDFGDSSGTLISRALVVTHTYLEPGPVTAQVVLQAAIPLTSCGSSPV
PGTTDGHRPTAEAPNTTAGQVPTTEVVGTTPGQAPTAEPSGTTSVQVPTTE
VISTAPVQMPTAESTGMTPEKVPVSEVMGTTLAEMSTPEATGMTPAEVSIV
VLSGTTAAQVTTTEWVETTARELPIPEPEGPDASSIMSTESITGSLGPLLD
GTATLRLVKRQVPLDCVLYRYGSFSVTLDIVQGIESAEILQAVPSGEGDAF
ELTVSCQGGLPKEACMEISSPGCQPPAQRLCQPVLPSPACQLVLHQILKGG
SGTYCLNVSLADTNSLAVVSTQLIMPGQEAGLGQVPLIVGILLVLMAVVLA
SLIYRRRLMKQDFSVPQLPHSSSHWLRLPRIFCSCPIGENSPLLSGQQV.

In myasthenia gravis, a main antigen is acetylcholine receptor.
In pemphigus vulgaris and variants, main antigens are desmoglein 3, 1 and 4; other antigens include pemphaxin, desmocollins, plakoglobin, perplakin, desmoplakins, and acetylcholine receptor.
In bullous pemphigoid, main antigens include BP180 and BP230; other antigens include plectin and laminin 5.
In dermatitis herpetiformis Duhring, main antigens include endomysium and tissue transglutaminase.
In epidermolysis bullosa acquisita, a main antigen is collagen VII.
In systemic sclerosis, main antigens include matrix metalloproteinase 1 and 3, the collagen-specific molecular chaperone heat-shock protein 47, fibrillin-1, and PDGF receptor; other antigens include Scl-70, U1 RNP, Th/To, Ku, Jo1, NAG-2, centromere proteins, topoisomerase I, nucleolar proteins, RNA polymerase I, II and III, PM-Slc, fibrillarin, and B23.
In mixed connective tissue disease, a main antigen is U1snRNP.
In Sjogren's syndrome, the main antigens are nuclear antigens SS-A and SS-B; other antigens include fodrin, poly(ADP-ribose) polymerase and topoisomerase, muscarinic receptors, and the Fc-gamma receptor IIIb.
In systemic lupus erythematosus, main antigens include nuclear proteins including the “Smith antigen,” SS-A, high mobility group box 1 (HMGB1), nucleosomes, histone proteins and double-stranded DNA (against which auto-antibodies are made in the disease process).
In Goodpasture's syndrome, main antigens include glomerular basement membrane proteins including collagen IV.
In rheumatic heart disease, a main antigen is cardiac myosin.
In autoimmune polyendocrine syndrome type 1 antigens include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.
In neuromyelitis optica, a main antigen is AQP4.
   Aquaporin-4, including an exogenously obtained form useful in the compositions of the disclosure, has the following sequence (UNIPROT P55087):

(SEQ ID NO: 21)
MSDRPTARRWGKCGPLCTRENIMVAFKGVWTQAFWKAVTAEFLAMLIFVLL
SLGSTINWGGTEKPLPVDMVLISLCFGLSIATMVQCFGHISGGHINPAVTV
AMVCTRKISIAKSVFYIAAQCLGAIIGAGILYLVTPPSVVGGLGVTMVHGN
LTAGHGLLVELIITFQLVFTIFASCDSKRTDVTGSIALAIGFSVAIGHLFA
INYTGASMNPARSFGPAVIMGNWENHWIYWVGPIIGAVLAGGLYEYVFCPD
VEFKRRFKEAFSKAAQQTKGSYMEVEDNRSQVETDDLILKPGVVHVIDVDR
GEEKKGKDQSGEVLSSV.

In uveitis, main antigens include Retinal S-antigen or “S-arrestin” and interphotoreceptor retinold binding protein (IRBP) or retinol-binding protein 3.
   S-arrestin, including an exogenously obtained form useful in the compositions of the disclosure, has the following sequence (UNIPROT P10523):

(SEQ ID NO: 22)
MAASGKTSKS EPNHVIFKKI SRDKSVTIYL GNRDYIDHVS
QVQPVDGVVL VDPDLVKGKK VYVTLTCAFR YGQEDIDVIG
LTFRRDLYFS RVQVYPPVGA ASTPTKLQES LLKKLGSNTY
PFLLTFPDYL PCSVMLQPAP QDSGKSCGVD FEVKAFATDS
TDAEEDKIPK KSSVRLLIRK VQHAPLEMGP QPRAEAAWQF
FMSDKPLHLA VSLNKEIYFH GEPIPVTVTV TNNTEKTVKK
IKAFVEQVAN VVLYSSDYYV KPVAMEEAQE KVPPNSTLTK
TLTLLPLLAN NRERRGIALD GKIKHEDTNL ASSTIIKEGI
DRTVLGILVS YQIKVKLTVS GFLGELTSSE VATEVPFRLM
HPQPEDPAKE SYQDANLVFE EFARHNLKDA GEAEEGKRDK
NDVDE.

IRBP, including an exogenously obtained form useful in the compositions of the disclosure, has the following sequence (UNIPROT P10745):

(SEQ ID NO: 23)
MMREWVLLMSVLLCGLAGPTHLFQPSLVLDMAKVLLDNYCFPENLLGMQEA
IQQAIKSHEILSISDPQTLASVLTAGVQSSLNDPRLVISYEPSTPEPPPQV
PALTSLSEEELLAWLQRGLRHEVLEGNVGYLRVDSVPGQEVLSMMGEFLVA
SHVWGNLMGTSALVLDLRHCTGGQVGIPYIISYLHPGNTILHVDTIYNRPS
NTTTEIWTLPQVLGERYGADKDVVVLTSSQTRGVAEDIAHILKQMRRAIVV
GERTGGGALDLRKLRIGESDFFFTVPVSRSLGPLGGGSQTWEGSGVLPCVG
TPAEQALEKALAILTLRSALPGVVHCLQEVLKDYYTLVDRVPTLLQHLASM
DFSTVVSEEDLVTKLNAGLQAASEDPRLLVRAIGPTETPSWPAPDAAAEDS
PGVAPELPEDEAIRQALVDSVFQVSVLPGNVGYLRFDSFADASVLGVLAPY
VLRQVWEPLQDTEHLIMDLRHNPGGPSSAVPLLLSYFQGPEAGPVHLFTTY
DRRTNITQEHFSHMELPGPRYSTQRGVYLLTSHRTATAAEEFAFLMQSLGW
ATLVGEITAGNLLHTRTVPLLDTPEGSLALTVPVLTFIDNHGEAWLGGGVV
PDAIVLAEEALDKAQEVLEFHQSLGALVEGTGHLLEAHYARPEVVGQTSAL
LRAKLAQGAYRTAVDLESLASQLTADLQEVSGDHRLLVFHSPGELVVEEAP
PPPPAVPSPEELTYLIEALFKTEVLPGQLGYLRFDAMAELETVKAVGPQLV
RLVWQQLVDTAALVIDLRYNPGSYSTAIPLLCSYFFEAEPRQHLYSVFDRA
TSKVTEVWTLPQVAGQRYGSHKDLYILMSHTSGSAAEAFAHTMQDLQRATV
IGEPTAGGALSVGIYQVGSSPLYASMPTQMAMSATTGKAWDLAGVEPDITV
PMSEALSIAQDIVALRAKVPTVLQTAGKLVADNYASAELGAKMATKLSGLQ
SRYSRVTSEVALAEILGADLQMLSGDPHLKAAHIPENAKDRIPGIVPMQIP
SPEVFEELIKFSFHTNVLEDNIGYLRFDMFGDGELLTQVSRLLVEHIWKKI
MHTDAMIIDMRFNIGGPTSSIPILCSYFFDEGPPVLLDKIYSRPDDSVSEL
WTHAQVVGERYGSKKSMVILTSSVTAGTAEEFTYIMKRLGRALVIGEVTSG
GCQPPQTYHVDDTNLYLTIPTARSVGASDGSSWEGVGVTPHVVVPAEEALA
RAKEMLQHNQLRVKRSPGLQDHL.

In the embodiments where the antigen is a foreign antigen against which an unwanted immune response can be developed, such as food antigens, specific antigens can be:
from peanut: conarachin (Ara h 1), allergen II (Ara h 2), arachis agglutinin, conglutin (Ara h 6);
   conarachin, for example has the sequence identified as UNIPROT Q6PSU6
from apple: 31 kda major allergen/disease resistance protein homolog (Mal d 2), lipid transfer protein precursor (Mal d 3), major allergen Mal d 1.03D (Mal d 1);
from milk: α-lactalbumin (ALA), lactotransferrin; from kiwi: actinidin (Act c 1, Act d 1), phytocystatin, thaumatin-like protein (Act d 2), kiwellin (Act d 5);
from egg whites: ovomucoid, ovalbumin, ovotransferrin, and lysozyme;
from egg yolks: livetin, apovitillin, and vosvetin;
from mustard: 2S albumin (Sin a 1), 11S globulin (Sin a 2), lipid transfer protein (Sin a 3), profilin (Sin a 4);
from celery: profilin (Api g 4), high molecular weight glycoprotein (Api g 5);
from shrimp: Pen a 1 allergen (Pen a 1), allergen Pen m 2 (Pen m 2), tropomyosin fast isoform;
from wheat and/or other cereals: high molecular weight glutenin, low molecular weight glutenin, alpha-, gamma- and omega-gliadin, hordein, secalin and/or avenin;
   peptides/epitopes useful in the compositions of the disclosure for treating Celiac Disease include some or all of the following sequences, individually in a composition of Formula 1, together in a cocktail of compositions of Formula 1, or fused in one or more compositions of Formula 2:

DQ-2 relevant, Alpha-gliadin “33-mer” native:
(SEQ ID NO: 24)
LQLQPFPQPQLPYPQPQLPYPQPQLPYPQPQPF
DQ-2 relevant, Alpha-gliadin “33-mer” deamidated:
(SEQ ID NO: 25)
LQLQPFPQPELPYPQPELPYPQPELPYPQPQPF
DQ-8 relevant, Alpha-gliadin:
(SEQ ID NO: 26)
QQYPSGQGSFQPSQQNPQ
DQ-8 relevant, Omega-gliadin (wheat, U5UA46):
(SEQ ID NO: 27)
QPFPQPEQPFPW

from strawberry: major strawberry allergy Fra a 1-E (Fra a 1); and
from banana: profilin (Mus xp 1).

In the embodiments where the antigen is a foreign antigen against which an unwanted immune response is developed, such as to animal, plant and environmental antigens, specific antigens can, for example, be: cat, mouse, dog, horse, bee, dust, tree and goldenrod, including the following proteins or peptides derived from:
weeds, (including ragweed allergens amb a 1, 2, 3, 5, and 6, and Amb t 5; pigweed Che a 2 and 5;
and other weed allergens Par j 1, 2, and 3, and Par o 1);
grass (including major allergens Cyn d 1, 7, and 12; Dac g 1, 2, and 5; Hol|1.01203; Lol p 1, 2, 3, 5, and 11; Mer a 1; Pha a 1; Poa p 1 and 5);
pollen from ragweed and other weeds (including curly dock, lambs quarters, pigweed, plantain, sheep sorrel, and sagebrush), grass (including Bermuda, Johnson, Kentucky, Orchard, Sweet vernal, and Timothy grass), and trees (including catalpa, elm, hickory, olive, pecan, sycamore, and walnut);
dust (including major allergens from species Dermatophagoides pteronyssinus, such as Der p 1, 2, 3, 4, 5, 6, 7, 8, 9, 10, 11, 14, 15, 18, 20, 21, and 23; from species Dermatophagoides farina, such as Der f 1, 2, 3, 6, 7, 10, 11, 13, 14, 15, 16, 18, 22, and 24; from species Blomia tropicalis such as Blo t 1, 2, 3, 4, 5, 6, 10, 11, 12, 13, 19, and 21; also allergens Eur m 2 from Euroglyphus maynei, Tyr p 13 from Tyrophagus putrescentiae, and allergens Bla g 1, 2, and 4; Per a 1, 3, and 7 from cockroach);
pets (including cats, dogs, rodents, and farm animals; major cat allergens include Fel d 1 through 8, cat IgA, BLa g 2, and cat albumin; major dog allergens include Can f 1 through 6, and dog albumin);
bee stings, including major allergens Api m 1 through 12; and
fungus, including allergens derived from, species of Aspergillus and Penicillium, as well as the species Alternaria alternata, Davidiella tassiana, and Trichophyton rubrum.

As will be appreciated by those skilled in the art, a patient can be tested to identify an antigen against which an unwanted immune response has developed, and a protein, peptide or the like can be developed based on that antigen and incorporated as X in a composition of the present disclosure.

### Sialated Antigens, Antibodies, Antibody Fragments

Following are examples of antigens, antibodies, antibody fragments having sialylation that can be removed to leave glycosylation specifically targeting the ASGPR: follicle stimulating hormone (FSH), human chorionic gonadotropin (HCG), luteinizing hormone (LH), osteopontin, thyroid stimulating hormone (TSH), agalsidase alfa, agalsidase beta (FABRAZYME®; Genzyme), epoetin alfa and epoetin beta, follitropin alfa (GONAL-FR; Merck/Serono) and follitropin beta (FOLLISTIM®; Schering-Plough), insulin growth factor binding protein 6 (IGFBP-6), lutropin alfa (LUVERIS®; Merck/Serono), transforming growth factor β1, antithrombin (ATryn®/TROMBATE-III®; Genzyme/Talecris Biotherapeutics), thyrotropin alfa (THYROGEN®; Genzyme), lenograstim, sargramostim (LEUKINE®; Genzyme), interleukin-3, prourokinase, lymphotoxin, C1-esterase inhibitor (Berinert®; CSL), IgG-like antibodies, interferon beta, coagulation factor VIIa (NOVOSEVEN®; Novo Nordisk), coagulation factor VIII (moroctocog alfa), coagulation factor IX (nonacog alfa) (BENEFIX®; Wyeth), and the p55 tumor necrosis receptor fusion protein. (See: Byrne et al., Drug Discovery Today, Vol 12, No. 7/8, pages 319-326, April 2007 and Sola et al., BioDrugs. 2010; 24 (1): 9-21). Pharmaceutically relevant proteins that have previously been hyperglycosylated and can be desialylated for hepatocyte-ASGPR targeting include: interferon alfa and gamma, luteinizing hormone, Fv antibody fragments, asparaginase, cholinesterase, darbepoetin alfa (AraNESP®; Amgen), thrombopoietin, leptin, FSH, IFN-α2, serum albumin, and corifollitropin alfa.

Proteins with glycans that do not normally terminate in sialic acids, including proteins produced in bacteria or yeast (such as arginase, some insulins, and uricase) would not be amenable to desialylation.

Those skilled in the art will appreciate that publicly available references, such as UNIPROT, disclose the presence and location of sites for desialylation on most if not all antigens, antibodies, antibody fragments and ligands of interest.

### Antibodies and Peptide Ligands

In the embodiments employing an antibody, antibody fragment or ligand, such moieties are chosen to specifically bind a targeted circulating protein or peptide or antibody, and result in hepatic uptake of the circulating targeted moiety, possibly as an adduct with the targeting moiety, ultimately resulting in the clearance and inactivation of the circulating targeted moiety. For example, liver-targeted Factor VIII will bind and clear circulating Factor VIII antibodies. Procedures for the identification of such moieties will be familiar to those skilled in the art.

### Linkers

The linkers employed in the compositions of the present disclosure (“Y” in Formula 1) can include N-hydroxysuccinamidyl linkers, malaemide linkers, vinylsulfone linkers, pyridyl di-thiol-poly(ethylene glycol) linkers, pyridyl di-thiol linkers, n-nitrophenyl carbonate linkers, NHS-ester linkers, nitrophenoxy poly(ethylene glycol) ester linkers and the like.

One particular group of linkers Formula Y′-CMP below (where Y′ indicates the remaining portion of the linker and R⁹ and Z are as defined). More particularly, in the group of linkers including Formula Y′-CMP, the R⁹ substituent is an ethylacetamido group, and even more particularly the ethylacetamido is conjugated with C1 of N-acetylgalactosamine.

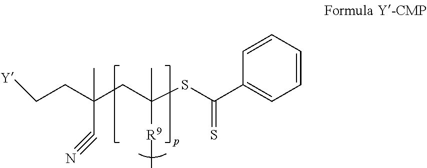

Di-thiol-containing linkers, particularly disulfanylethyl carbamate-containing linkers (named including a free amine of X, otherwise named a “disulfanyl ethyl ester” without including the free amine of X) are particularly advantageous in the present compositions as having the ability to cleave and release an antigen in its original form once inside a cell, for example as illustrated below (where Y′ indicates the remaining portion of the linker and X′ and Z are as defined).

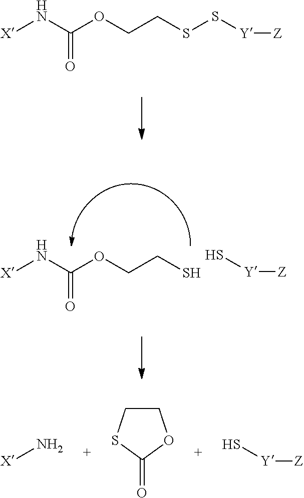

Particularly with regard to the linkers illustrated below in Formula Ya through Formula Yp:
the the left bracket “(” indicates the bond between X and Y;
the right or bottom bracket “)” indicates the bond between Y and Z;
n is an integer representing a mixture including from about 1 to 100, particularly about 8 to 90 (e.g., 7, 8, 9, 10, 11, 12, 13, 14, 15, 16, 17, 18, 19, 20, 25, 30, 35, 40, 45, 50, 55, 60, 65 70, 75, 80, 85, 90 or 95), more particularly about 40 to 80 (e.g., 39, 40, 43, 45, 46, 48, 50, 52, 53, 55, 57, 60, 62, 65, 66, 68, 70, 73, 75, 78, 80 or 81) ethylene glycol groups, where the mixture typically encompasses the integer specified as n±10%;
p is an integer representing a mixture including from about 2 to 150, particularly about 20 to 100 (e.g., 18, 19, 20, 25, 30, 35, 40, 45, 50, 55, 60, 65 70, 75, 80, 85, 90, 95, 100 or 105) and more particularly about 30 to 40 (e.g., 28, 29, 30, 31, 32, 33, 34, 35, 36, 37, 38, 39, 40, 41, 42, 43 or 44), where the mixture typically encompasses the integer specified as p±10%;
q is is an integer representing a mixture including from about 1 to 44, particularly about 3 to 20 (e.g., 3, 4, 5, 6, 7, 8, 9, 10, 11, 12, 13, 14, 15, 16, 17, 18, 19, 20, 21 or 22) and more particularly about 4 to 12 (e.g., 4, 5, 6, 7, 8, 9, 10, 11, 12 or 13), where the mixture typically encompasses the integer specified as q±10%; and
R⁸ is —CH₂—(“methyl”) or —CH₂—CH₂—C(CH₃)(CN)— (“1-cyano-1-methyl-propyl” or “CMP”).
R⁹ is a direct bond or —CH₂—CH₂—NH—C(O)— (an ethylacetamido group or “EtAcN”), as illustrated in the following structures of Formula 1 (where the EtAcN group is shown and the rest of the linker is referred to as Y′):

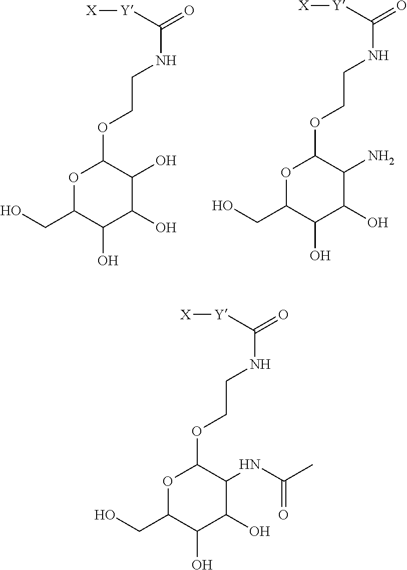

and Z is galactose, galactosamine or N-acetylgalactosamine conjugated at C1.

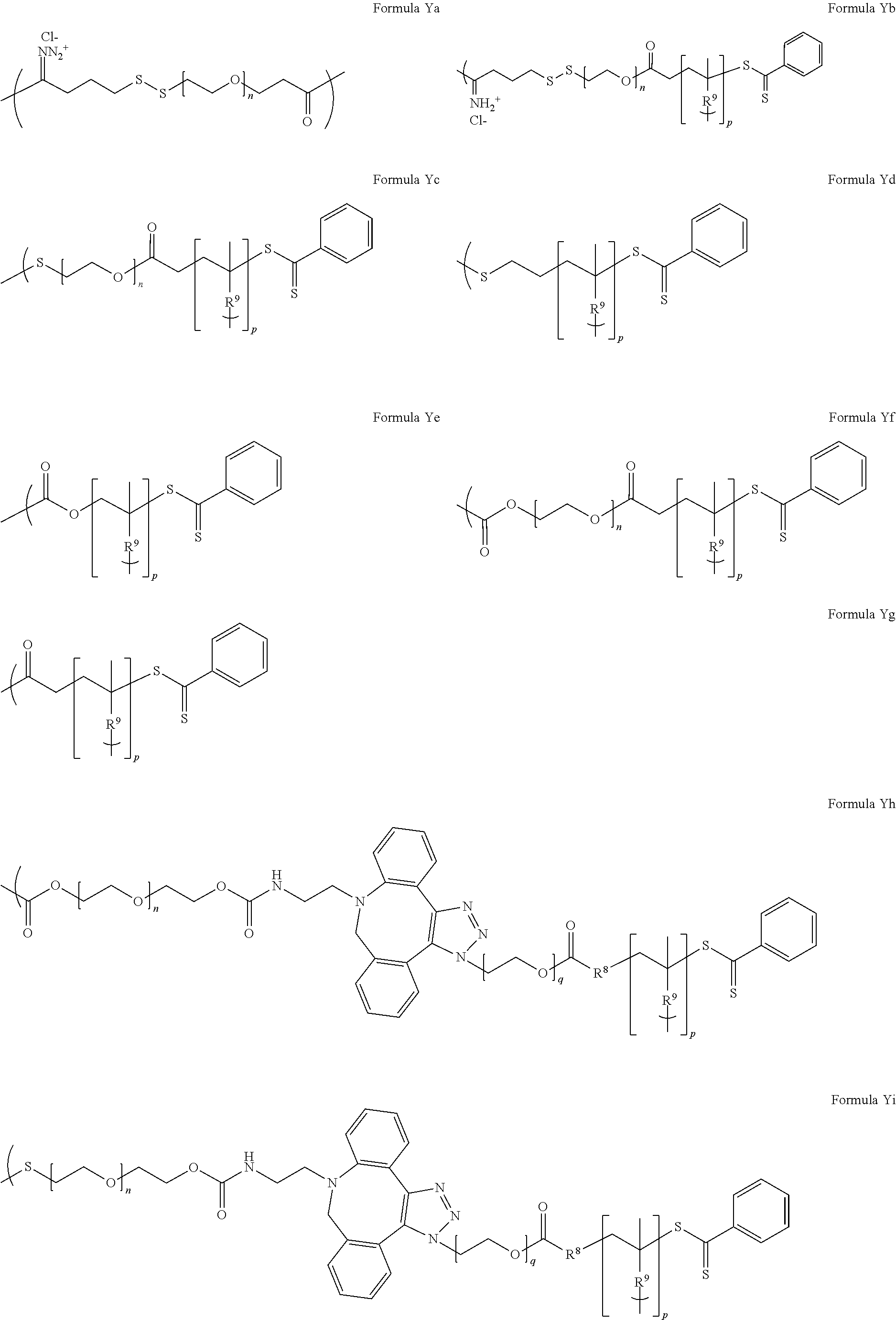

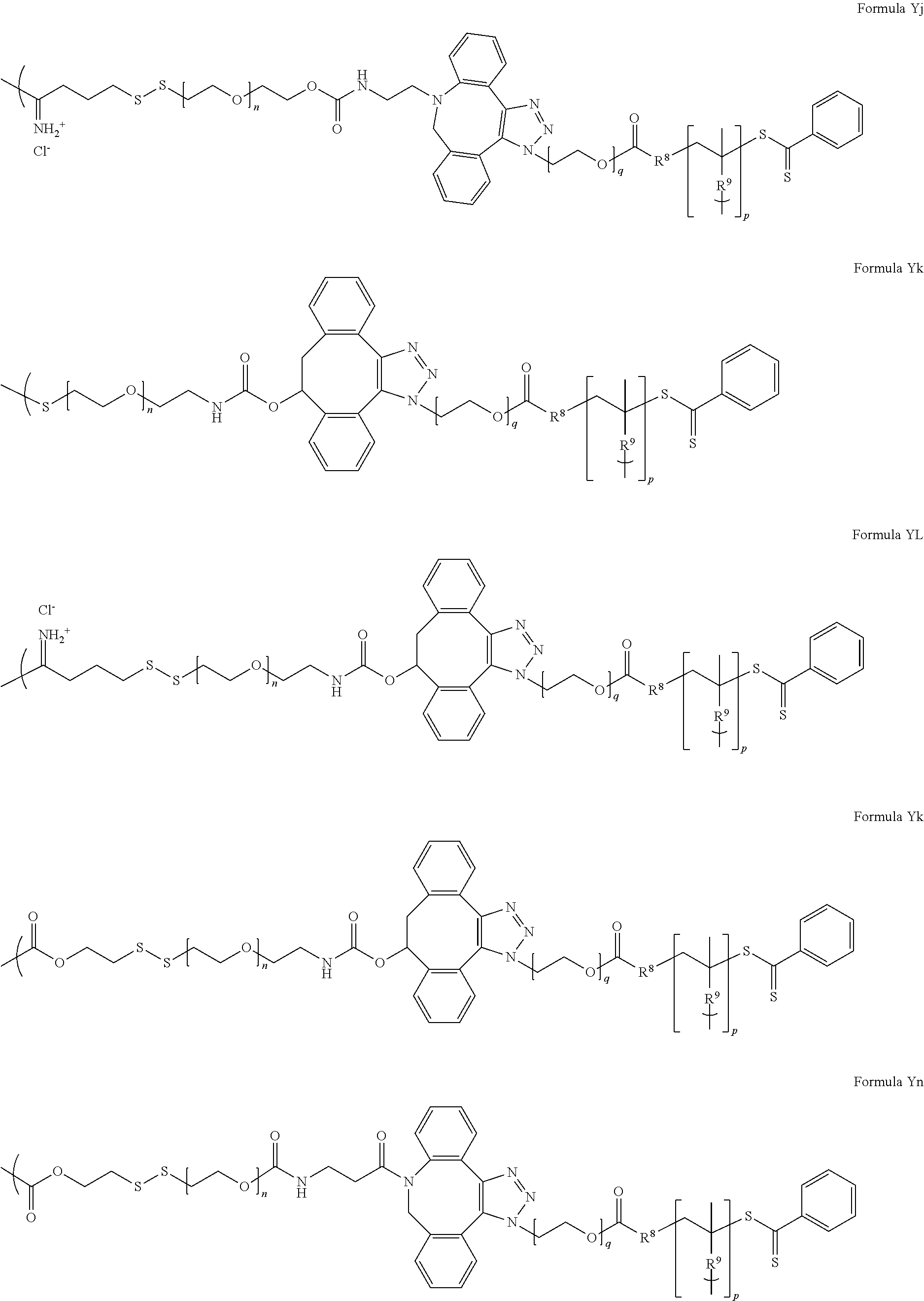

(Linkers of Formula Yn can be synthesized via certain precursors that render Yn particularly suitable for conjugation to hydrophobic antigens.)

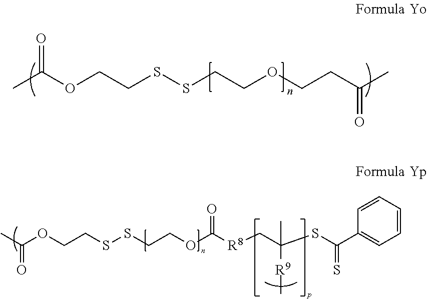

The linkers shown above as Formulae Yh through Yn are synthesized as isomers that are employed without separation. For example, the linkers of Formulae Yh, Yi, Yj and Yn will be a mixture of the 8,9-dihydro-1H-dibenzo[b,f][1,2,3]triazolo[4,5-d]azocin-8yl and 8,9-dihydro-3H-dibenzo[b,f][1,2,3]triazolo[4,5-d]azocin-8yl structures illustrated below:

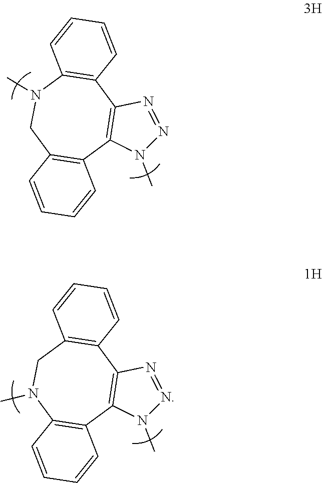

The linkers of Formulae Yk, YL and Ym will be a mixture of the 8,9-dihydro-1H-dibenzo[3,4:7,8]cycloocta[1,2-d][1,2,3]triazol-8-yl and 8,9-dihydro-1H-dibenzo[3,4:7,8] cycloocta [1,2-d][1,2,3]triazol-9-yl structures illustrated below:

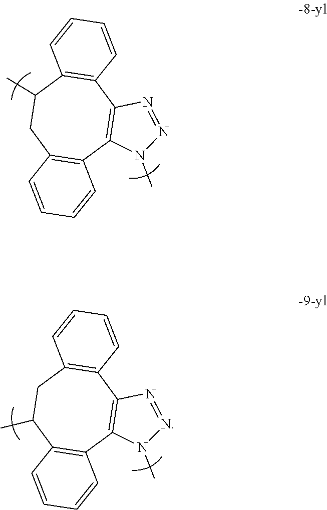

The presence of such isomeric mixtures does not impair the functionality of the compositions employing such linkers.

### Galactosylating Moieties

The galactosylating moieties employed in the compositions of the present disclosure serve to target the compositions to liver cells (for example, specifically binding hepatocytes) and can be selected from: galactose, galactosamine or N-acetylgalactosamine. It has been reported that ASGPR affinity can be retained while modifying either side of galactose's C3/C4-diol anchor (Mamidyala, Sreeman K., et al., J. Am. Chem. Soc. 2012, 134, 1978-1981), therefore the points of conjugation are particularly at C1, C2 and C6.

Particular galactosylating moieties include galactose conjugated at C1 or C6, galactosamine conjugated at C2, and N-acetylgalactosamine conjugated at C6. Other particular galactosylating moieties include N-acetylgalactosamine conjugated at C2, more particularly conjugated to a linker bearing an R⁹ substituent that is CH₂. Still other particular galactosylating moieties include galactose, galactosamine or N-acetylgalactosamine conjugated at C1, more particularly conjugated to a linker bearing an R⁹ substituent that is an ethylacetamido group.

### ASGPR Targeting Antibodies

The ASGPR-specific antibodies employed in the compositions of the present disclosure also serve to target compositions of the disclosure to liver cells and can be selected from commercially available products, such as: Anti-Asialoglycoprotein Receptor 1 antibody (ab42488) from Abcam plc, Cambridge, UK and ASGPR^{1/2} (FL-291) (sc28977) from Santa Cruz Biotechnology, Inc., Dallas, TX. Alternatively, such antibodies can be expressed using any of a number of published sequences, such as the Dom26h-196-61, single-domain anti-ASGPR antibody:

(SEQ.ID.No: 6)
EVQLLESGGGLVQPGGSLRLSCAASGFTFEKYAMAWVRQAPGKGLEWVS
RISARGVTTYYADSVKGRFTISRDNSKNTLYLQMNSLRAEDTAVYYCAS
HKRHEHTRFDSWGQGTLVTVSS

[Coulstock E, et al., (2013) “Liver-targeting of interferon-alpha with tissue-specific domain antibodies.” PLOS One. 8 (2):e57263 and US 2013/0078216], or such a sequence having conservative substitutions. The above-referenced US patent application discloses liver-targeting molecules such as DOM26h-196-61 for delivering certain therapeutics [including interferon (interferon alpha 2, interferon alpha 5, interferon alpha 6, or consensus interferon), Ribavirin, Nexavar/Sorafenib, Erbitus/Cetuximab, Avastatin/bevacizumab, and Herceptin/trastuzumab] for the treatment of liver diseases. The compositions of matter corresponding to Formula 2 employing DOM26h-196-61 or another liver-targeting molecule described in US 2013/0078216 do not include interferon (interferon alpha 2, interferon alpha 5, interferon alpha 6, or consensus interferon), Ribavirin, Nexavar/Sorafenib, Erbitus/Cetuximab, Avastatin/bevacizumab, and Herceptin/trastuzumab within their scope.

New sequences for an antibody, antibody fragment, or peptide that specifically targets ASGPR can be discovered using various methods known by those skilled in the art. These methods can include, but are not limited to, vaccination technology, hybridoma technology, library display technologies (including bacterial and phage platforms), endogenous repertoire screening technologies, directed evolution and rational design.

### Nomenclature

The compositions of Formula 1 can be named using a combination of IUPAC and trivial names. For example, a compound corresponding to Formula 1 where X is a cyclobutyl moiety (shown instead of an antigen for illustrative purposes), Y is Formula Ya, m is 1, n is 4 and Z is N-acetylgalactosamin-2-yl:

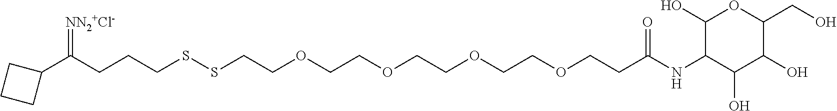

can be named (Z)-(21-cyclobutyl-1-oxo-1-((2,4,5-trihydroxy-6-(hydroxymethyl)tetrahydro-2H-pyran-3-yl)amino)-4,7,10,13-tetraoxa-16, 17-dithiahenicosan-21-ylidene)triaz-1-yn-2-ium chloride, SO the corresponding composition of the disclosure where X is tissue transglutaminase can be named (Z)-(21-(tissue transglutaminase)-1-oxo-1-((2,4,5-trihydroxy-6-(hydroxymethyl)tetrahydro-2H-pyran-3-yl)amino)-4,7,10,13-tetraoxa-16, 17-dithiahenicosan-21-ylidene)triaz-1-yn-2-ium chloride. The corresponding composition of the disclosure where X′ is tissue transglutaminase, m is 2, n is 4 and Z′ is N-acetylgalactosamin-2-yl can be named (3Z)-((tissue transgultaminase)-1,3-diylbis(1-oxo-1-((2,4,5-trihydroxy-6-(hydroxymethyl)tetrahydro-2H-pyran-3-yl)amino)-4,7,10, 13-tetraoxa-16, 17-dithiahenicosan-21-yl-21-ylidene))bis(triaz-1-yn-2-ium) chloride.

In the interest of simplification, the compositions of Formula 1 can be named using an alternative naming system by reference to X and correspondence to one of Formulae 1a to 1p (as illustrated in the reaction schemes) followed by recitation of the integers for variables m, n, p and/or q, R⁸, R⁹ and identification of the galactosylating moiety and the position at which it is conjugated. Under this system, the composition of Formula 1a where X is ovalbumin, m is 2, n is 4 and Z is N-acetylgalactosamin-2-yl can be named “F1a-OVA-m₂-n₄-2NAcGAL.”

Similarly, the following composition of Formula 1

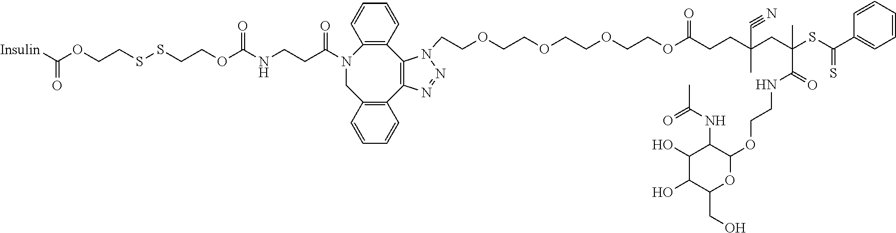

can be named “2-((2-(((3-(3-(22-((3-acetamido-4,5-dihydroxy-6-(hydroxymethyl)tetrahydro-2H-pyran-2-yl)oxy)-16-cyano-16, 18-dimethyl-13, 19-dioxo-18-((phenylcarbonothioyl)thio)-3,6,9,12-tetraoxa-20-azadocosyl)-3,9-dihydro-8H-dibenzo[b,f][1,2,3]triazolo[4,5-d]azocin-8-yl)-3-oxopropyl) carbamoyl)oxy)ethyl) disulfanyl)ethyl insulin carboxylate.” The isomer:

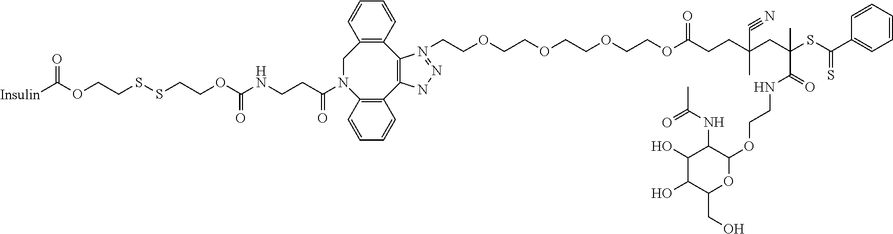

can be named “2-((2-(((3-(1-(22-((3-acetamido-4,5-dihydroxy-6-(hydroxymethyl)tetrahydro-2H-pyran-2-yl)oxy)-16-cyano-16, 18-dimethyl-13, 19-dioxo-18-((phenylcarbonothioyl)thio)-3,6,9,12-tetraoxa-20-azadocosyl)-1,9-dihydro-8H-dibenzo[b,f][1,2,3]triazolo[4,5-d]azocin-8-yl)-3-oxopropyl) carbamoyl)-oxy)ethyl) disulfanyl)ethyl insulin carboxylate” (bold lettering highlights added for convenience in identifying the difference between the formal names). Employing the naming system asopted for the present disclosure, both isomers can be named “F1n-insulin-m₁-n₁-p₁-q₄-CMP-EtAcN-1NAcGAL” where CMP indicates that R⁸ is 1-cyano-1-methyl-propyl, EtAcN indicates that R⁹ is ethylacetamido and 1NAcGAL indicates Z″ is N-acetylgalactosamine conjugated at C1. Absence of the abbreviation EtAcN before the designation for Z would indicate that R⁹ is a direct bond.

In the compositions of Formula 2, left-to-right orientation of should not be taken as specifying N to C ordering absent specific indication to the contrary. For example, the compound of Formula 2 where m′ is 1, m″ is 0, X is Ovalbumin, Y is Gly₃Ser and Z is Anti-ASGPR Dom26h-196-61, can be named OVA-Gly₃Ser-DOM and read as covering both of the following:

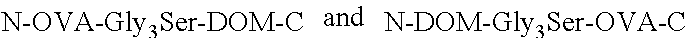

The compositions of Formula 2, for example where m′ is 1, m″ is 0, X is Ovalbumin, Y is Gly₃Ser and Z is Anti-ASGPR Dom26h-196-61 (having a purification tag attached via an additional linker) can be named as follows:

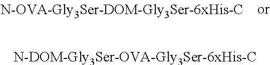

where the C′ terminal Gly₃Ser-6×His group represents an amino acid sequence that facilitates isolation and purification.

The composition of Formula 2 where m′ is 1, m″ is 1, each X is Factor VIII, each Y is Gly₃Ser and Z is Anti-ASGPR Dom26h-196-61 (having a purification tag attached via an additional linker) can be named FVIII-Gly₃Ser-DOM-Gly₃Ser-FVIII-Gly₃Ser-6×His and covers both of the following:

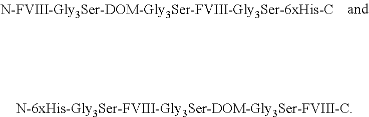

The composition of Formula 2 where m′ is 3, m″ is 0, one, the three X antigens, respectively, are Alpha-gliadin “33-mer” deamidated (SEQ ID NO:25), Alpha-gliadin (SEQ ID NO:26) and Omega-gliadin (SEQ ID NO:27), each Y is a linker having an immunoproteosome cleavage site (“IPC”), and Z is Anti-ASGPR Dom26h-196-61 can be named:

Alpha-gliadin “33-mer” deamidated-IPC-Alpha-gliadin-IPC-Omega-gliadin-IPC-DOM.

### Preparation of the Compositions of the Disclosure

The compositions of Formula 1 can be prepared, for example, by adjusting the procedures described in Zhu, L., et al., Bioconjugate Chem. 2010, 21, 2119-2127. Syntheses of certain compositions of Formula 1 are also described below with reference to Reaction Schemes 1 to 14. Other synthetic approaches will be apparent to those skilled in the art.

Formula 101 (below) is an alternative representation of X

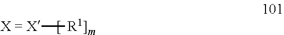

where R¹ is a free surface amino (—NH₂) or thiol (—SH) moiety positioned on X's three-dimensional structure so as to be accessible for conjugation to a linker, and X′ represents the remainder of X excluding the identified free amino group(s) [(X″ is used in the reaction schemes to represent the remainder of X excluding free thiol group(s)]. Depending upon the identity of X, there will be at least one (the N-terminal amine) and can be multiple R¹ groups (predominantly from lysine residues or cysteine residues that are not involved in disulfide bonding), as represented by m, which is an integer from about 1 to 100, more typically 1 or from about 4 to 20, and most typically 1 to about 10.

Variables employed in the reaction schemes are as defined above, and additionally include the following, which should be understood to have these meanings absent any specific indication otherwise with respect to a particular reaction scheme or step.
R² is OH or a protecting group;
R³ is OH, NH₂, NHAc, a protecting group or NH-protecting group;
R⁴ is OH or a protecting group;
R⁵ is OH or a protecting group;
R⁶ is OH or a protecting group;
Z′ is galactose conjugated at C1 or C6, galactosamine conjugated at C2, or N-acetylgalactosamine conjugated at C6;
R⁸ is —CH₂— or —CH₂—CH₂—C(CH₃)(CN)—; and
R⁹ is a direct bond and Z″ is N-acetylgalactosamine conjugated at C2;
R⁹ is an ethylacetamido group and Z″ is galactose, galactosamine or N-acetylgalactosamine conjugated at C1.

### Synthetic Reaction Parameters

The terms “solvent”, “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present disclosure are inert organic solvents.

The term “q.s.” means adding a quantity sufficient to achieve a stated function, e.g., to bring a solution to the desired volume (i.e., 100%).

Isolation and purification of the compounds and intermediates described herein can be effected, if desired, by any suitable separation or purification procedure such as, for example, filtration, extraction, crystallization, column chromatography, thin-layer chromatography or thick-layer chromatography, centrifugal size exclusion chromatography, high-performance liquid chromatography, recrystallization, sublimation, fast protein liquid chromatography, gel electrophoresis, dialysis, or a combination of these procedures. Specific illustrations of suitable separation and isolation procedures can be had by reference to the examples hereinbelow. However, other equivalent separation or isolation procedures can, of course, also be used.

Unless otherwise specified (including in the examples), all reactions are conducted at standard atmospheric pressure (about 1 atmosphere) and ambient (or room) temperature (about 20° C.), at about pH 7.0-8.0.

Characterization of reaction products can be made by customary means, e.g., proton and carbon NMR, mass spectrometry, size exclusion chromatography, infrared spectroscopy, gel electrophoresis.

Reaction Scheme 1 illustrates the preparation of compositions of Formula 1 where Z can be galactose, galactosamine or N-acetylgalactosamine. In that regard and as defined above, Z′ as employed in Reaction Scheme 1 encompasses galactose conjugated at C1 and C6 and corresponding to the following structures according to Formula 1:

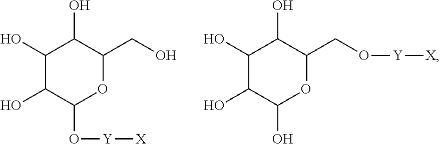

galactosamine conjugated at C2 and corresponding to the following structure according to Formula 1:

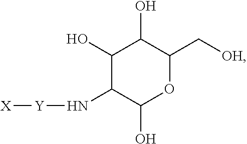

and N-acetylgalactosamine conjugated at C6 and corresponding to the following structure according to Formula 1:

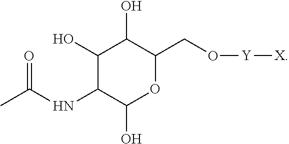

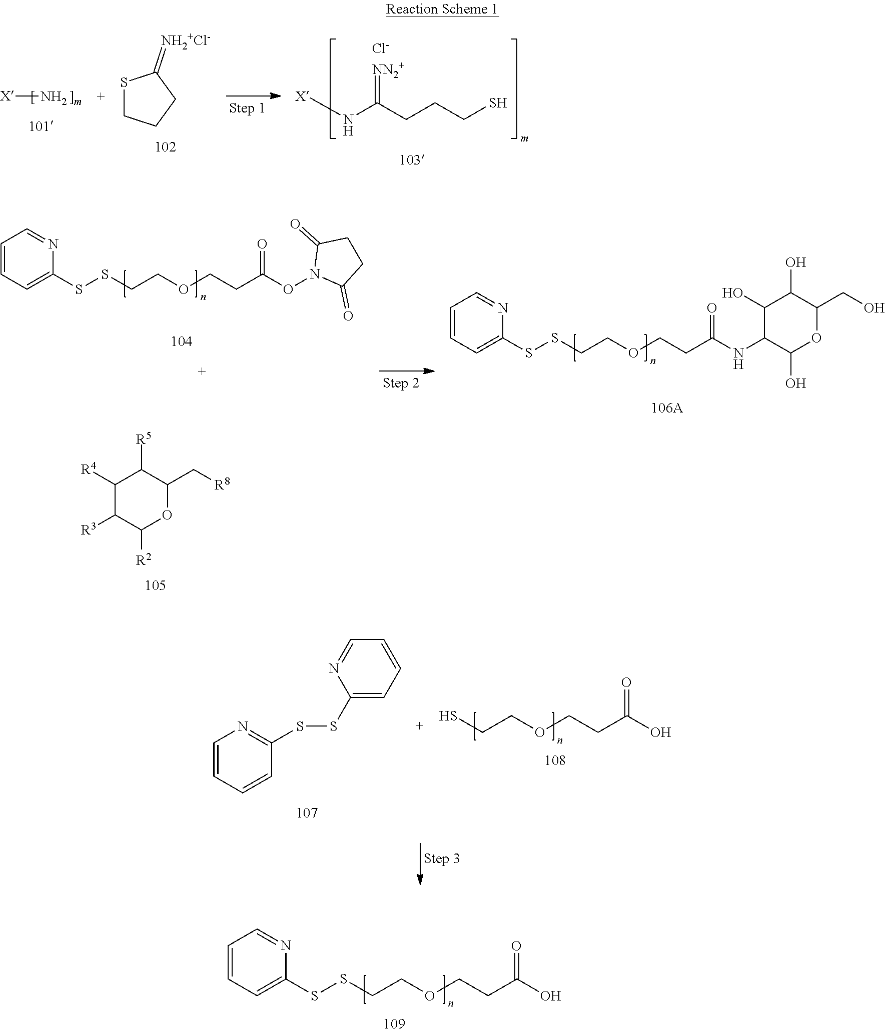

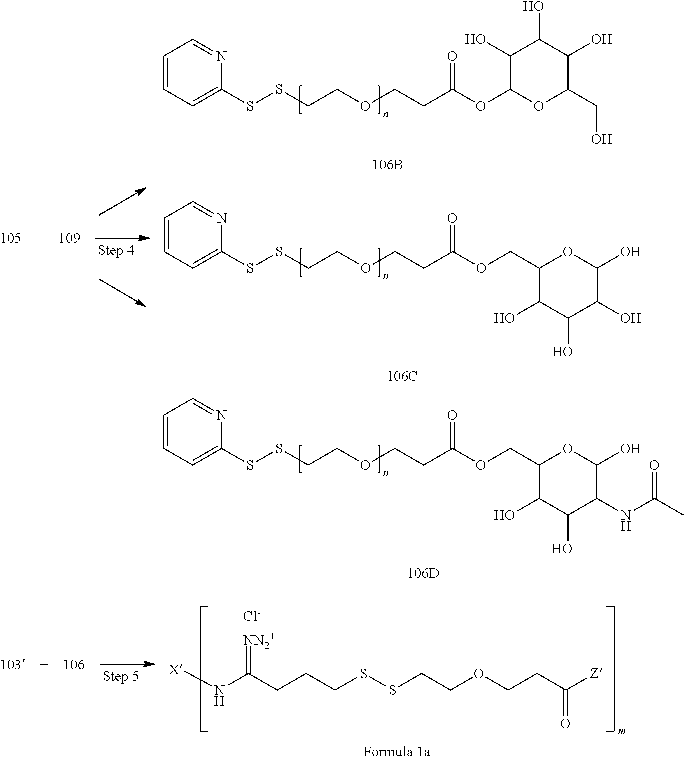

As illustrated above in Reaction Scheme 1, Step 1, surface thiol group(s) can be generated on an antigen, antibody, antibody fragment or ligand having free surface amino group(s) (Formula 101′) by contact with a Traut reagent (Formula 102) at a pH of about 8.0 for about 1 hour to give the Formula 103′, from which unreacted Traut's reagent is removed, e.g., via centrifugal size exclusion chromatography. The two structures shown below, illustrate the product of Reaction Scheme 1, Step 1, respectively showing the free surface amino group(s) originally found on X (i.e., Formula 103′ where X′ represents the remainder of X excluding the identified free surface amino groups) and omitting the free surface amino group(s) (i.e., Formula 103). This parallels the distinction illustrated as between X and Formula 101. The convention has been followed in the subsequent reaction schemes.

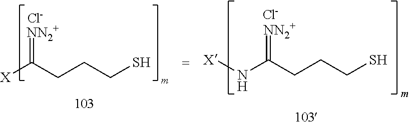

In Reaction Scheme 1, Step 2, a pyridyl di-thiol-poly(ethylene glycol)-NHS ester (Formula 104) is contacted with galactosamine (Formula 105 where R³ is NH₂ and R², R⁴, R⁵ and R⁶ are OH) with stirring at about pH 8 for about 1 hour to give the corresponding pyridyl di-thiol-poly(ethylene glycol)-sugar of Formula 106A, which can be used without further purification.

In Reaction Scheme 1, Step 3, 4,4′-dithiodipyridine (Formula 107) is contacted with a thiol-poly (ethylene glycol) propanoic acid (Formula 108) to give the corresponding pyridyl di-thiol-poly(ethylene glycol) propanoic acid (Formula 109).

In Reaction Scheme 1, Step 4, the acid of Formula 109 is contacted with a protected galactose or N-acetylgalactosamine of Formula 105 where R² is OH and R³, R⁴, R⁵ and R⁶ are protecting groups (“PG”), where R⁶ is OH and R², R³, R⁴ and R⁵ are PG, or where R⁶ is N-acetyl and R², R³, R⁴ and R⁵ are PG to give the corresponding pyridyl di-thiol-poly(ethylene glycol)-sugars of Formulae 106B, 106C and 106D, which can be used following de-protection.

In Reaction Scheme 1, Step 5, to a stirred solution of the product of Step 1 (Formula 103′) is added an excess (corresponding to the value of m) of the product of Step 2 or Step 4 (Formula 106, i.e., 106A, 106B, 106C or 106D) for about 1 hour, followed by centrifugal sized exclusion chromatography to remove any free remaining reactants to yield the corresponding product according to Formula 1a, respectively, Formula 1aA, Formula 1aB, Formula 1aC and Formula 1aD.

The compositions corresponding to Formula 1a can be named, respectively, e.g., as follows:

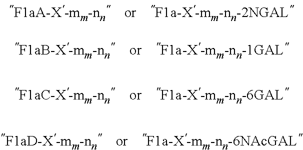

respectively, for products made employing an intermediate according to Formulae 106A-D.

Turning to Reaction Schemes 2-14 and for the purposes of the nomenclature employed therewith, except as expressly stated otherwise, Z″ refers to N-acetylgalactosamine conjugated at C2:

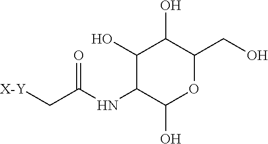

or to galactose, galactosamine or N-acetylgalactosamine conjugated at C1. It should be noted that the C1 conjugated compositions need to be protected during synthesis, for example by cyclizing the amine with the C3 hydroxyl and de-protecting following incorporation of the protected galactosamine into the adjacent portion of the linker.

The poly(galactose methacrylate) reactants of Formulae 201, 401, 501, 601, 701, 803 and 1401 can be prepared by methacrylating galactose, e.g., contacting galactosamine and methacrylate anhydride, followed by reversible addition-fragmentation chain transfer (RAFT) polymerization with a corresponding RAFT agent in the presence of azobisisobutyronitrile (AIBN) in a suitable solvent, starting with freeze-thaw cycles followed by heating at about 60-80° C., preferably 70° C. for about 5-8, preferably about 6 hours. The polymer can be precipitated in a lower alkanol, preferably methanol.

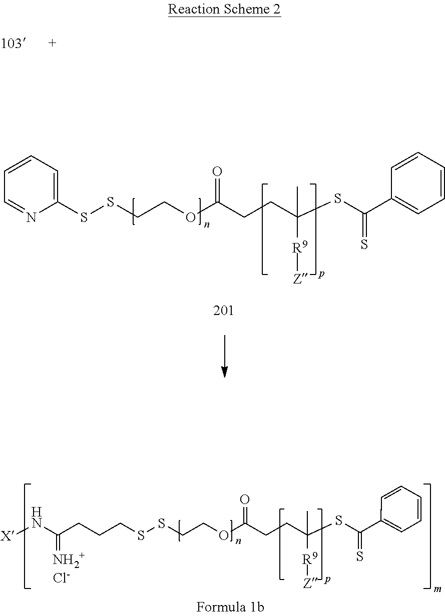

As illustrated in Reaction Scheme 2, an antigen, antibody, antibody fragment or ligand having free surface thiol group(s) prepared, e.g., as described with reference to Reaction Scheme 1, Step 1 (Formula 103′) is contacted with an excess (corresponding to the value of m) of a pyridyl di-thiol-poly(ethylene glycol) of Formula 201 for about 1 hour to yield the corresponding product according to Formula 1b.

The compositions of Formula 1b can be named as follows:

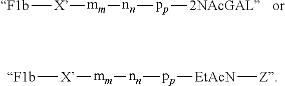

For example, the composition of Formula 1b where X′ is uricase, m is 1, n is 4, p is 4 and Z″ is N-acetylgalactosamine conjugated at C2 can be named “F1b-uricase-m₁-n₄-p₄-2NAcGAL” or “30-(uricase)-3,5,7,9-tetramethyl-12-oxo-1-phenyl-1-thioxo-3,5,7,9-tetrakis((2,4,5-trihydroxy-6-(hydroxymethyl)tetrahydro-2H-pyran-3-yl) carbamoyl)-13,16,19,22-tetraoxa-2,25,26-trithiatriacontan-30-iminium”.

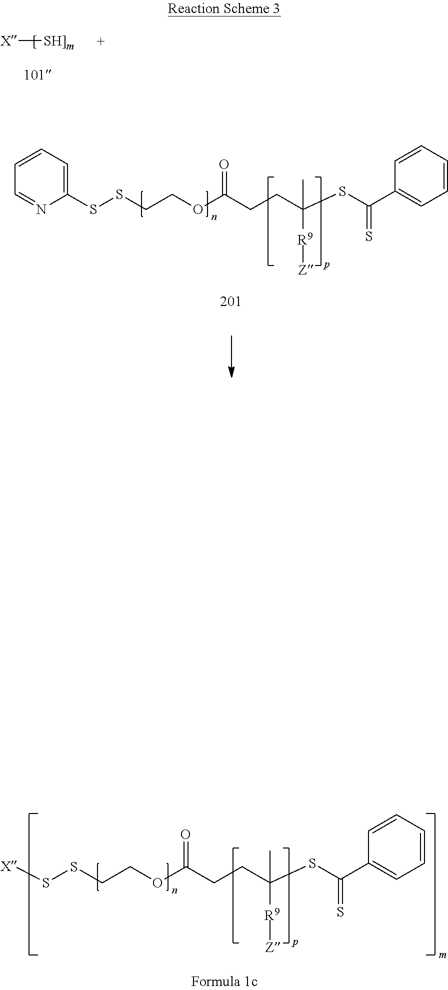

As illustrated in Reaction Scheme 3, an antigen, antibody, antibody fragment or ligand having native free surface thiol group(s) (cysteines) [Formula 101″ corresponding to Formula 101 and illustrating where X″, as the term will be subsequently employed, represents X excluding the identified free surface thiol group(s)] is contacted with an excess (corresponding to the value of m) of a pyridyl di-thiol-poly(ethylene glycol) of Formula 201 to yield the corresponding product according to Formula 1c.

The compositions corresponding to Formula 1c can be named as follows:

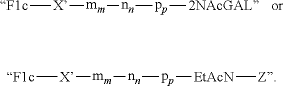

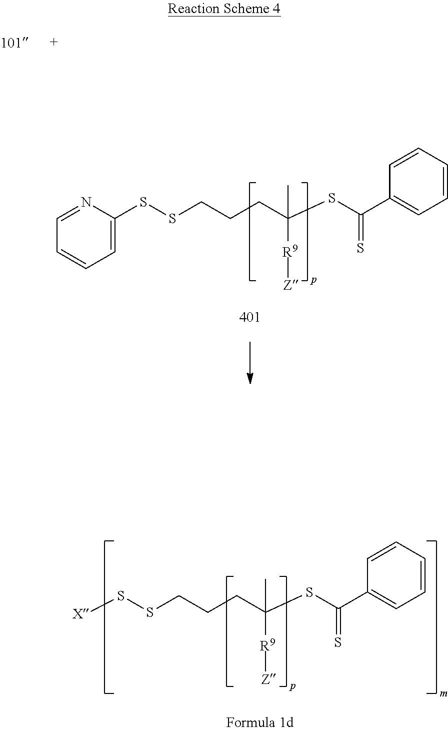

As illustrated in Reaction Scheme 4, an antigen, antibody, antibody fragment or ligand having native free surface thiol group(s) of Formula 101″ is contacted with an excess (corresponding to the value of m) of a pyridyl di-thiol of Formula 401 to yield the corresponding product according to Formula 1d.

The compositions corresponding to Formula 1d can be named as follows:

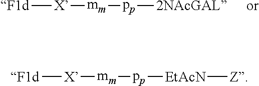

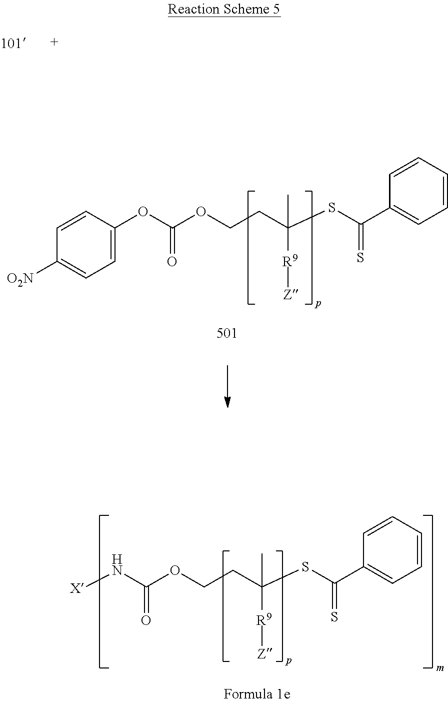

As illustrated in Reaction Scheme 5, an antigen, antibody, antibody fragment or ligand having native free surface amino group(s) of Formula 101′ is contacted with an excess (corresponding to the value of m) of a n-nitrophenyl carbonate of Formula 501 to yield the corresponding product according to Formula 1e.

The compositions corresponding to Formula 1e can be named as follows:

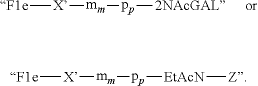

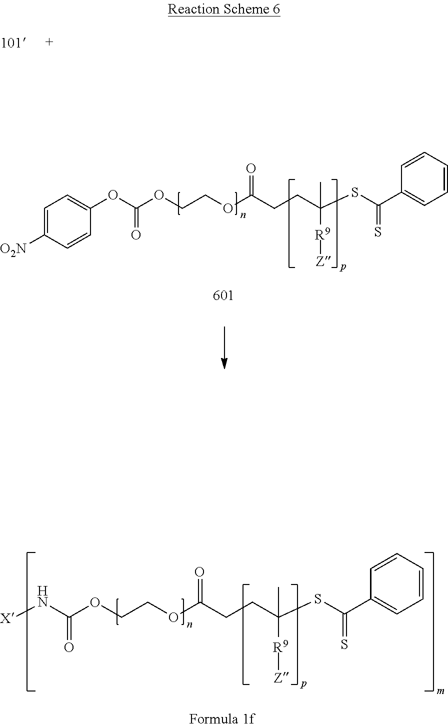

As illustrated in Reaction Scheme 6, an antigen, antibody, antibody fragment or ligand having native free surface amino group(s) of Formula 101′ is contacted with an excess (corresponding to the value of m) of a n-nitrophenyl carbonate poly(ethylene glycol) ester of Formula 601 to yield the corresponding product according to Formula 1f.

The compositions corresponding to Formula 1f can be named as follows:

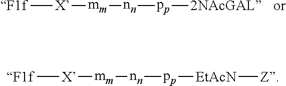

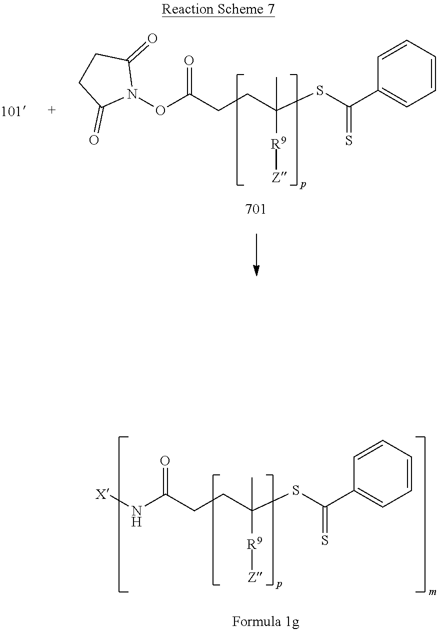

As illustrated in Reaction Scheme 7, an antigen, antibody, antibody fragment or ligand having native free surface amino group(s) of Formula 101′ is contacted with an excess (corresponding to the value of m) of a NHS-ester poly(ethylene glycol) ester of Formula 701 to yield the corresponding product according to Formula 1g.

The compositions corresponding to Formula 1g can be named as follows:

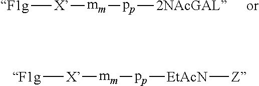

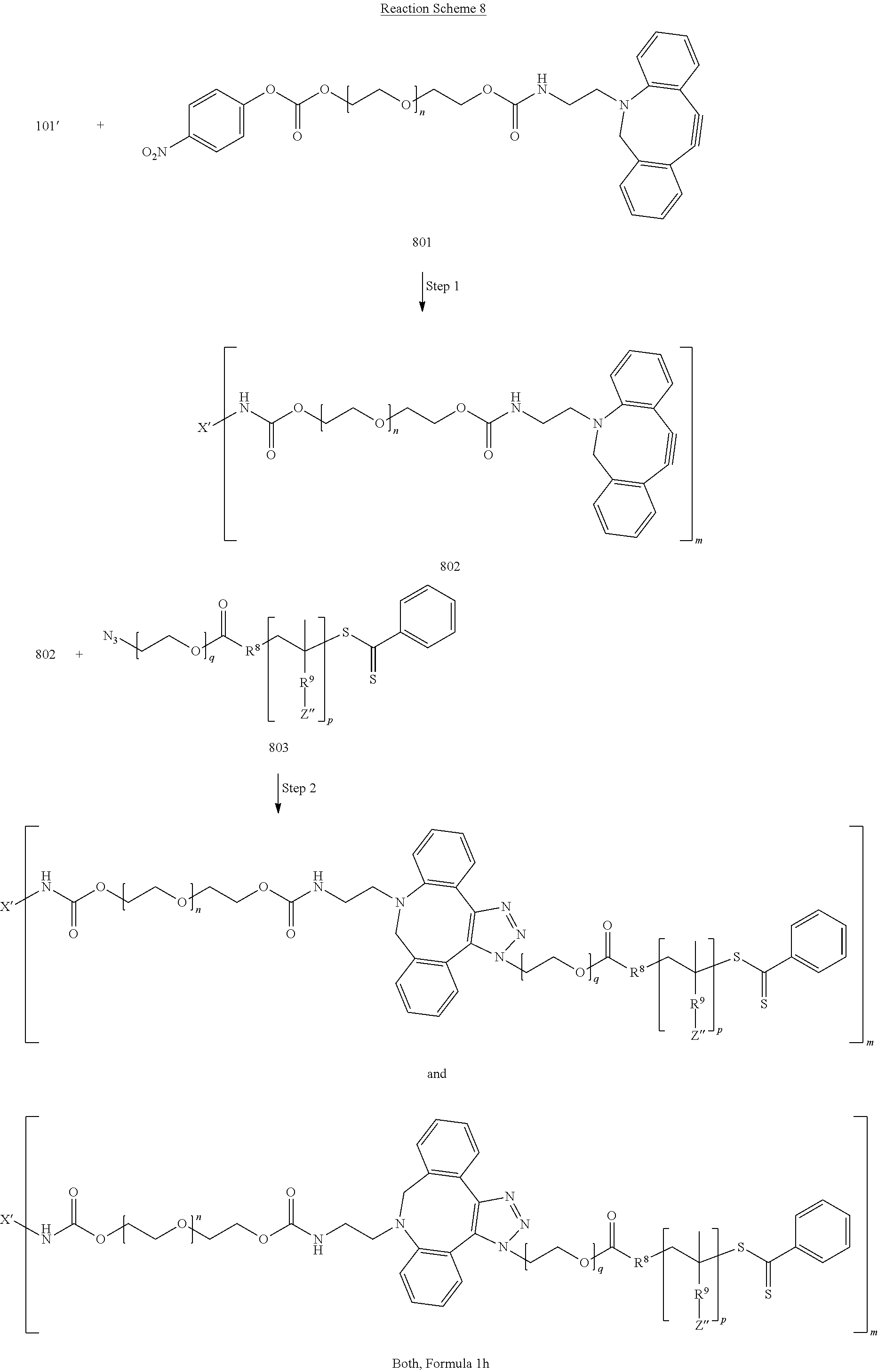

As illustrated in Reaction Scheme 8, Step 1, an antigen, antibody, antibody fragment or ligand having native free surface amino group(s) of Formula 101′ is contacted with an excess (corresponding to the value of m) of an amine-reactive linker for Click chemistry of Formula 801 to yield the corresponding product according to Formula 802.

In Reaction Scheme 8, Step 2, the product of Formula 802 is then contacted with an equivalent amount (again corresponding to the value of m) of a galactose (amine) polymer of Formula 803 to yield the corresponding isomeric product according to Formula 1 h. The two isomers, illustrated above, result from non-specific cyclization of the azide of Formula 803 with the triple bond of Formula 802. Such non-specific cyclization occurs in the synthesis of other compositions where Y is selected from Formulae Yh through Yn, but will not be illustrated in each instance.

The compositions corresponding to Formula 1h can be named as follows:

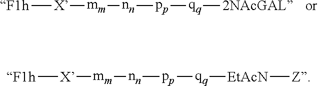

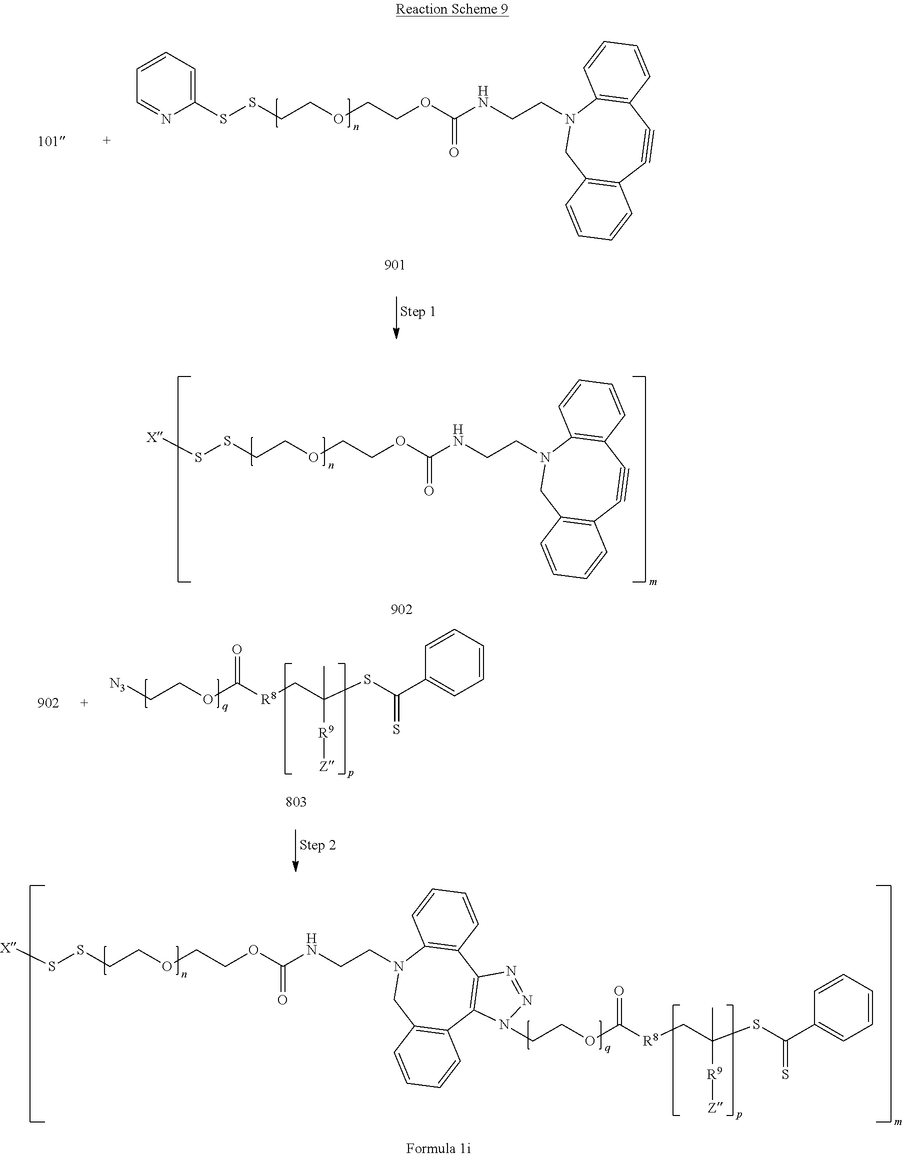

As illustrated in Reaction Scheme 9, Step 1, an antigen, antibody, antibody fragment or ligand having native free surface thiol group(s) of Formula 101″ is contacted with an excess (corresponding to the value of m) of a thiol-reactive linker for Click chemistry of Formula 901 to yield the corresponding product according to Formula 902″.

In Reaction Scheme 9, Step 2, the product of Formula 902″ is then contacted with an equivalent amount (again corresponding to the value of m) of a galactose (amine) polymer of Formula 803 to yield the corresponding isomeric product according to Formula 1i.

The compositions corresponding to Formula 1i can be named as follows:

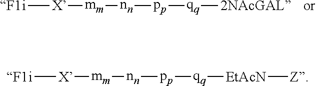

By following the procedures described with regard to Reaction Scheme 9, but substituting starting material 101″ with a compound of Formula 103′ (derivatized with the Traut reagent) there is obtained the corresponding isomeric product of Formula 1j as shown below.

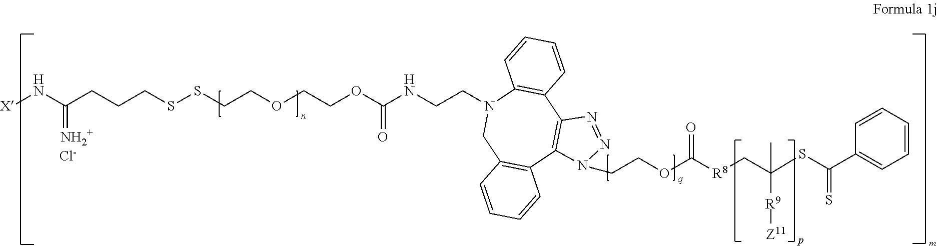

The compositions corresponding to Formula 1i can be named as follows:

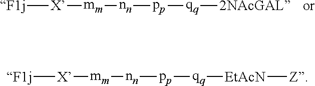

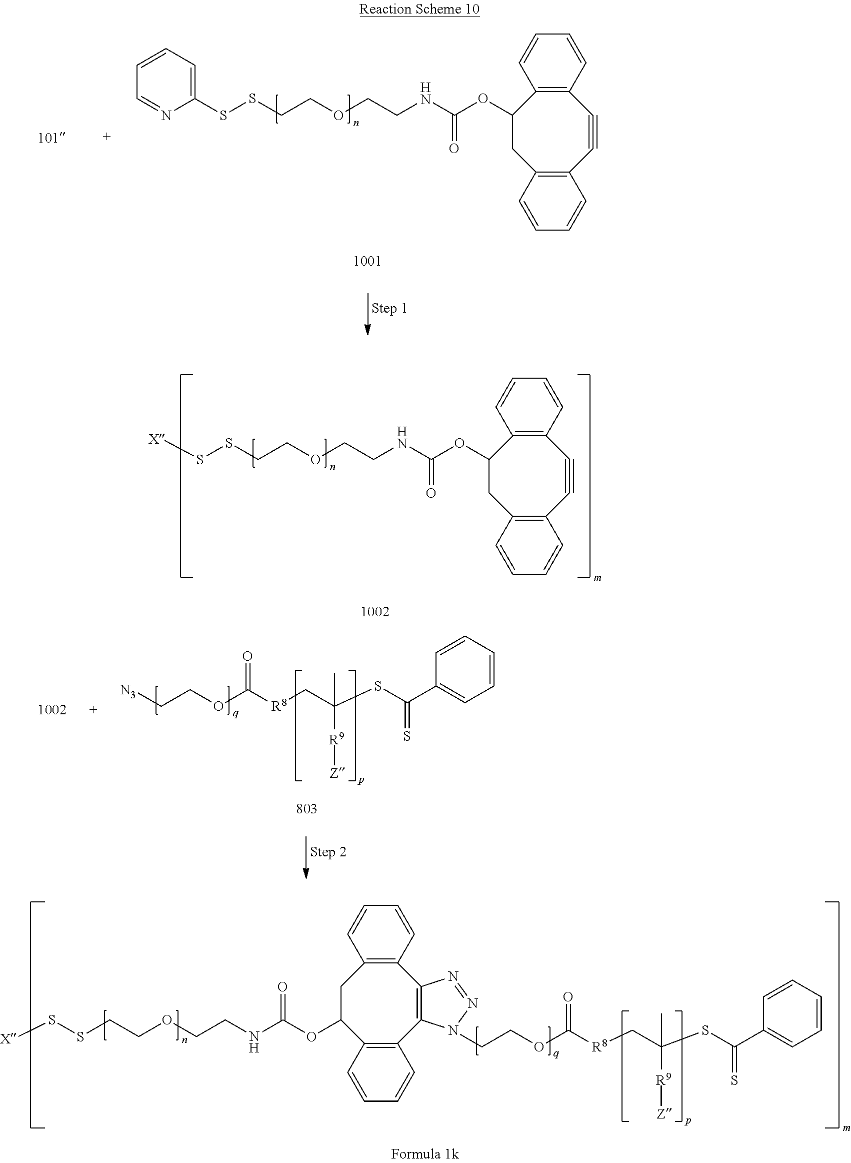

As illustrated in Reaction Scheme 10, Step 1, an antigen, antibody, antibody fragment or ligand having native free surface thiol group(s) of Formula 101″ is contacted with an excess (corresponding to the value of m) of a thiol-reactive linker for Click chemistry of Formula 1001 to yield the corresponding product according to Formula 1002.

In Reaction Scheme 10, Step 2, the product of Formula 1002 is then contacted with an equivalent amount (again corresponding to the value of m) of a galactose (amine) polymer of Formula 803 to yield the corresponding isomeric product according to Formula 1k.

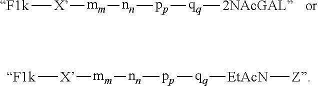

By following the procedures described with regard to Reaction Scheme 10, but substituting starting material 101″ with a compound of Formula 103′ (derivatized with the Traut reagent) there is obtained the corresponding isomeric product of Formula 1L as shown below.

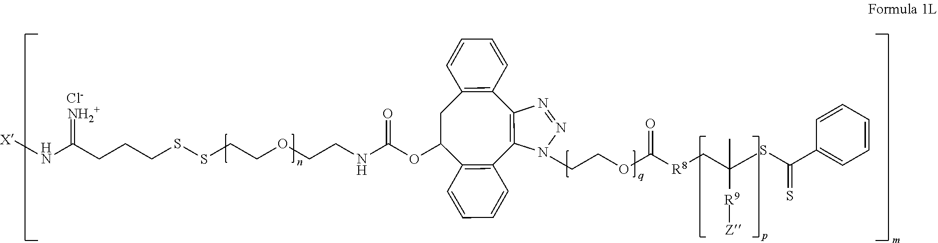

The compositions corresponding to Formula 1L can be named as follows:

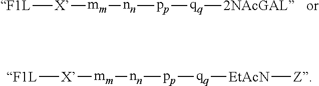

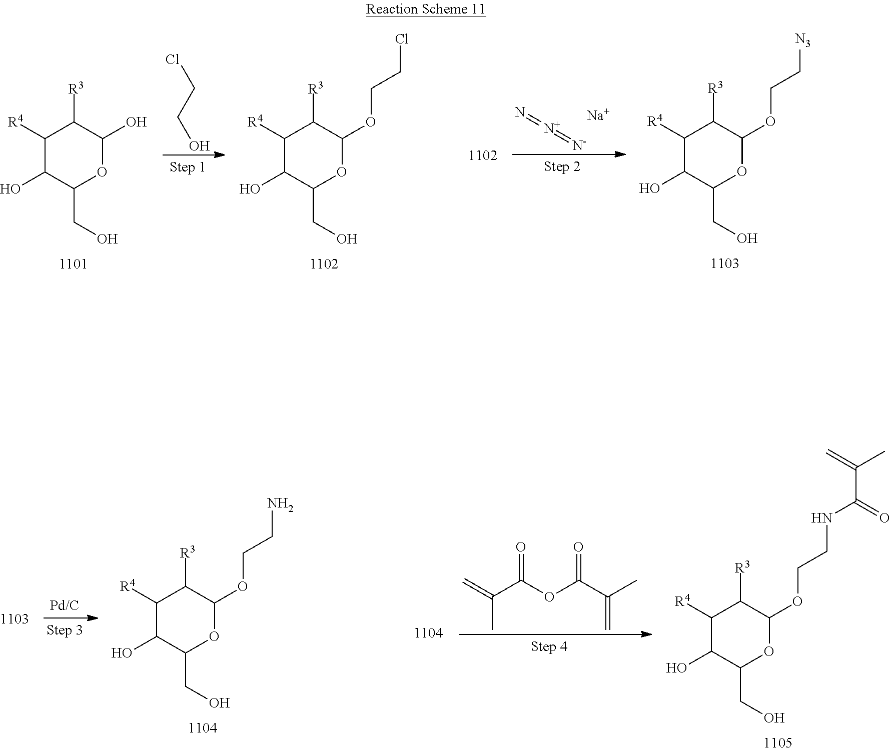

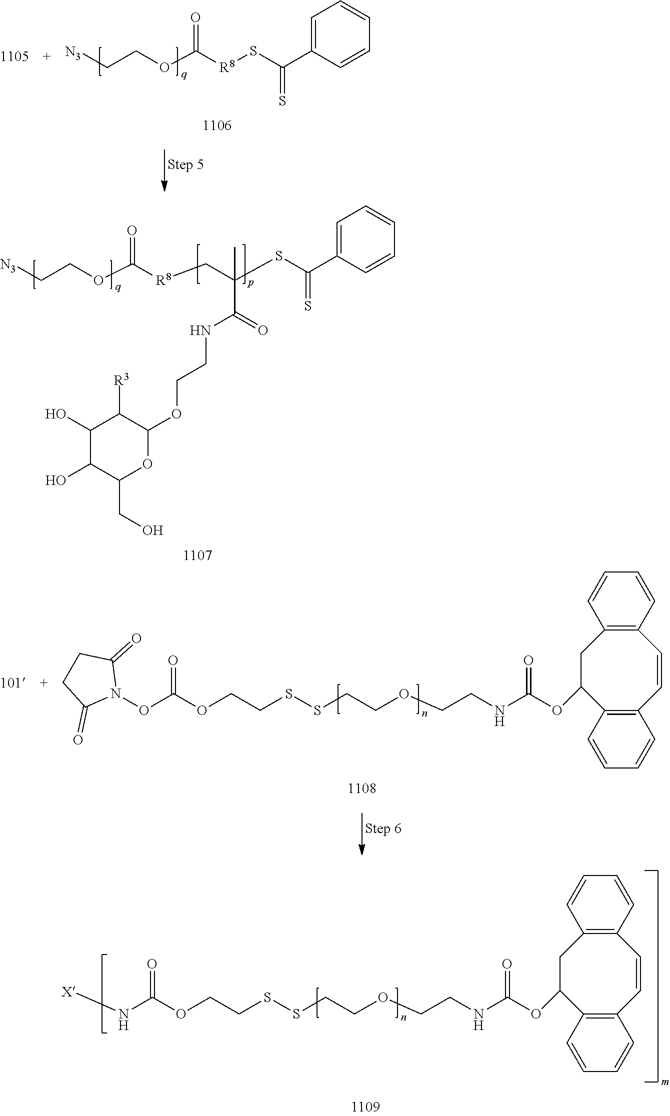

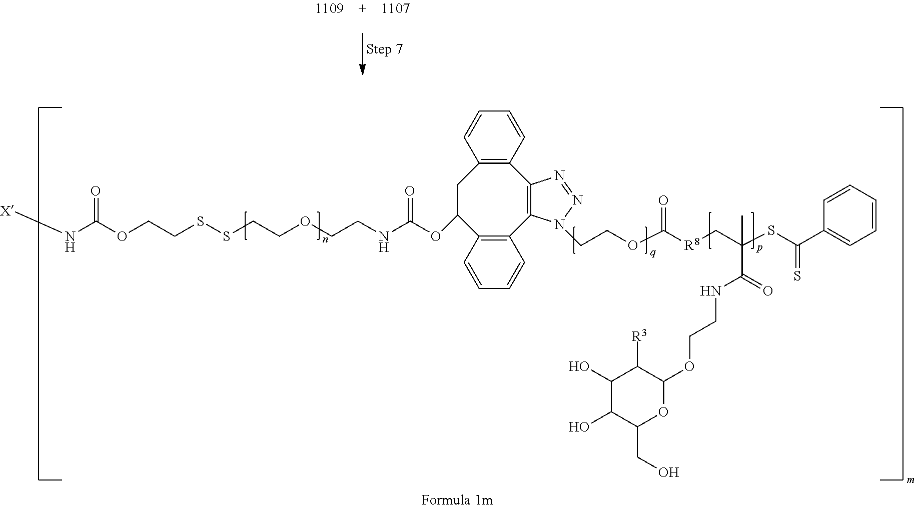

As illustrated in Reaction Scheme 11, Step 1, galactose, protected galactosamine or N-Acetyl-D-galactosamine (Formula1101 where R³ and R⁴ are OH, R³ is NH-protecting group (e.g., cyclized with R⁴) or R³ is NHAc and R⁴ is OH, respectively) is contacted with 2-chloroethan-1-ol followed by cooling and the dropwise addition of acetylchloride. The solution is warmed to room temperature and then heated to 70° C. for several hours. Ethanol is added to the crude product and the resulting solution is stirred in the presence of carbon and then filtered followed by solvent removal to yield the corresponding product of Formula 1102.

As illustrated in Reaction Scheme 11, Step 2, the product of Formula 1102 is added to an excess of sodium azide and heated to 90° C. for several hours, then filtered followed by solvent removal to yield the corresponding product of Formula 1103.

As illustrated in Reaction Scheme 11, Step 3, the product of Formula 1103 is added to a solution of palladium on carbon and ethanol, and stirred under hydrogen gas (3 atm) for several hours, then filtered followed by solvent removal to yield the corresponding product of Formula 1104.

As illustrated in Reaction Scheme 11, Step 4, the product of Formula 1104 is added to a solution of methacrylate anhydride. Triethylamine is added and the reaction stirred for 2 hours followed by solvent removal and isolation to yield the corresponding product of Formula 1105.

As illustrated in Reaction Scheme 11, Step 5, an azide-modified uRAFT agent (Formula 1106) is added to a solution of the product of Formula 1105 with azobisisobutyronitrile, subjected to 4 free-pump-thaw cycles and then stirred at 70° C. After several hours the corresponding polymer product of Formula 1107 is precipitated by addition of a lower alkanol followed by solvent removal. Where R³ is NH-protecting group (e.g., cyclized with R⁴) the protecting group(s) is (are) removed at this point.

As illustrated in Reaction Scheme 11, Step 6, an antigen, antibody, antibody fragment or ligand having native free surface amino group(s) of Formula 101′ is added to a pH 8.0 buffer and contacted with an excess (corresponding to the value of m) of a dioxopyrrolidine of Formula 1108 with stirring. After 1 hour unreacted Formula 1108 is removed and the resulting product of Formula 1109 is used without further purification.

As illustrated in Reaction Scheme 11, Step 7, the product of Formula 1107 is added to a pH 8.0 buffer, to which is added the product of Formula 1109. After stirring for 2 hours, the excess Formula 1107 is removed to yield the corresponding isomeric product of Formula 1m.

By substituting N-(2,4,5-trihydroxy-6-(hydroxymethyl)tetrahydro-2H-pyran-3-yl) methacrylamide for the product of Formula 1105 in Step 5 and continuing with Steps 6 and 7, the corresponding isomeric product of Formula 1m where Z″ is N-acetylgalactosamine conjugated at C2 are obtained.

The compositions corresponding to Formula 1m can be named as follows:

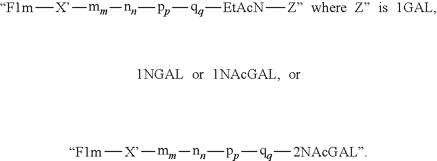

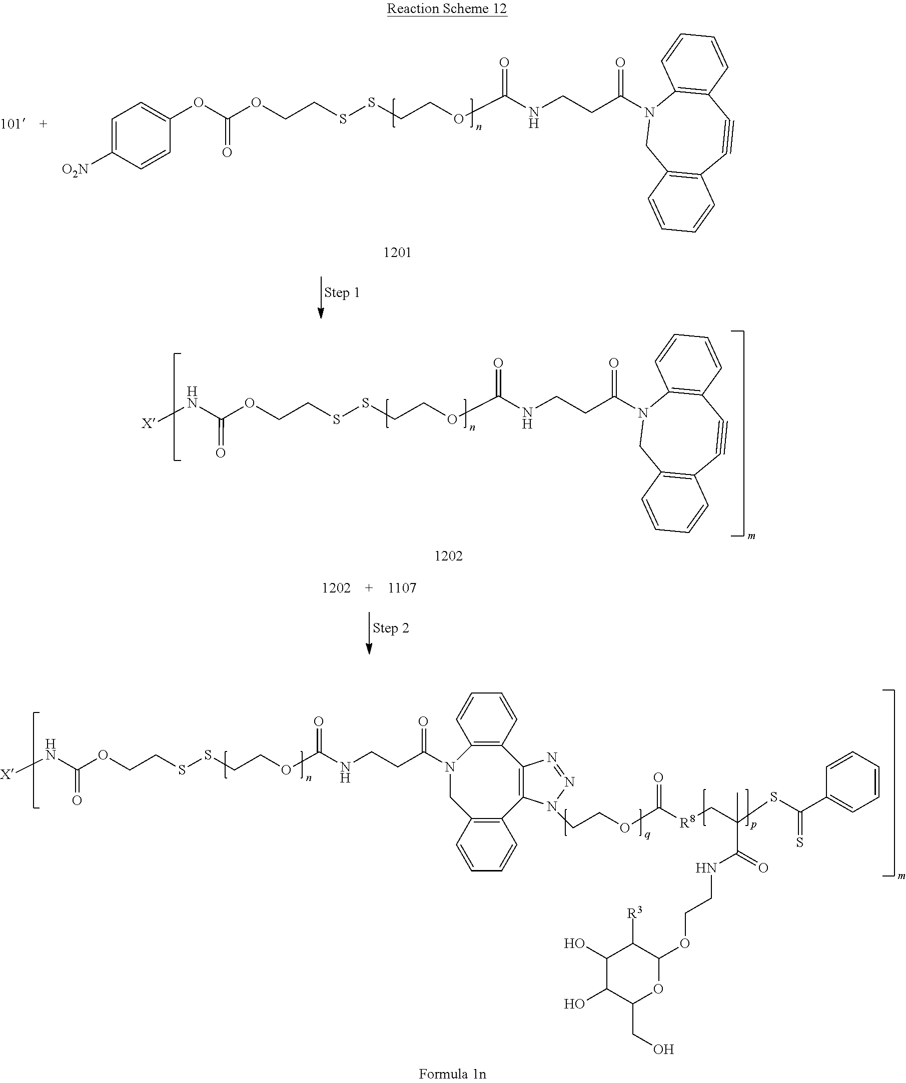

The synthetic approach of Reaction Scheme 12 is particularly suitable for hydrophobic antigens, antibodies, antibody fragments and ligands (e.g., Insulin) due to the use of organic solvents.

As illustrated in Reaction Scheme 12, Step 1, an antigen, antibody, antibody fragment or ligand having native free surface amino group(s) of Formula 101′ is dissolved in an organic solvent (e.g., DMF) containing triethylamine. To this is added an amount (corresponding to the value of m) of a compound of Formula 1201 followed by stirring and the addition of t-butyl methyl ether. The corresponding product of Formula 1202 is recovered as a precipitate.

The product of Formula 1202 is resuspended in the organic solvent and an amount (corresponding to the value of m) of Formula 1107 (obtained, e.g., as described with reference to Reaction Scheme 11) is added followed by stirring. The reaction product is precipitated via the addition of dichloromethane, followed by filtration and solvent removal. Purification (e.g., resuspension in PBS followed by centrifugal size exclusion chromatography yields the corresponding isomeric product of Formula 1n.

The compositions corresponding to Formula 1n can be named as follows:

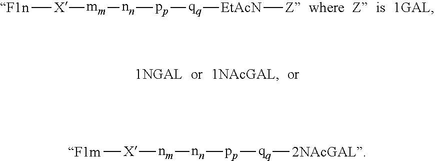

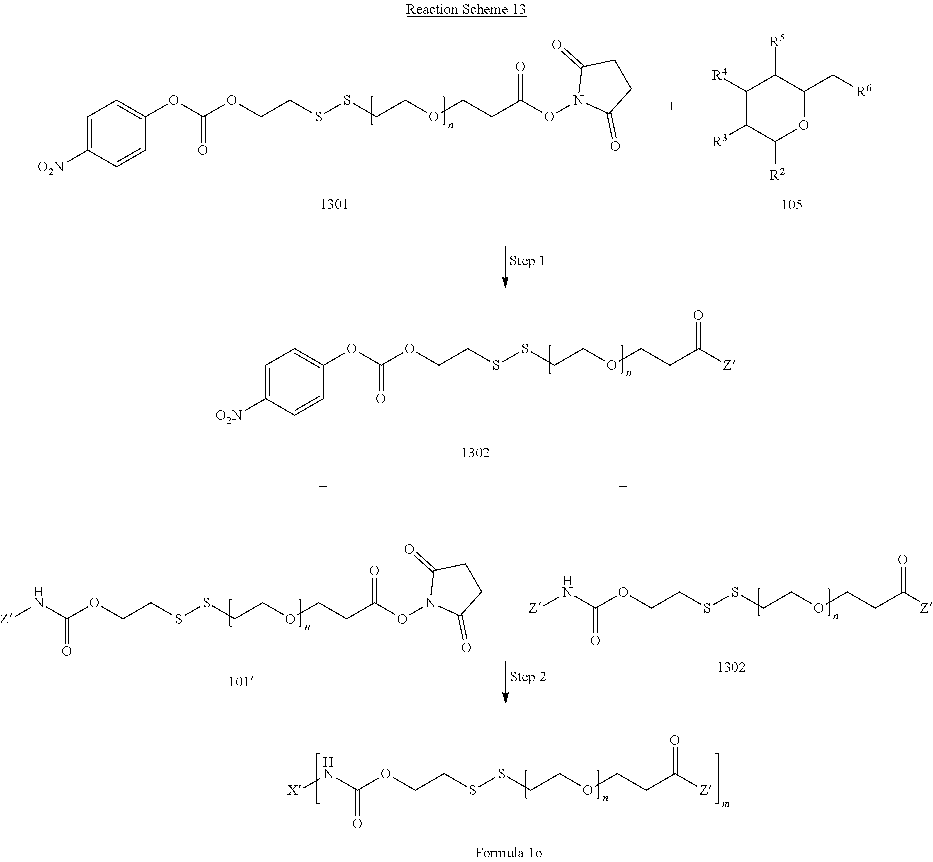

In Reaction Scheme 13, Step 1, a nitrophenoxycarbonyl-oxyalkyl di-thiol-poly(ethylene glycol)-NHS ester (Formula 1301) is contacted with galactose, galactosamine or N-acetylgalactosamine (Formula 105) to give the corresponding product of Formula 1302, along with the other two illustrated products, from which t desired nitrophenoxycarbonyl di-thiol-poly(ethylene glycol)-carboxyethyl galactose, galactosamine or N-acetylgalactosamine of Formula 1302 is isolated before proceeding to the next step.

As illustrated in Reaction Scheme 13, Step 2, an antigen, antibody, antibody fragment or ligand having native free surface amino group(s) of Formula 101′ is contacted with an excess (corresponding to the value of m) of the product of Formula 1302 to yield the corresponding product according to Formula 10.

The compositions corresponding to Formula 1o can be named as follows:

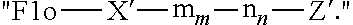

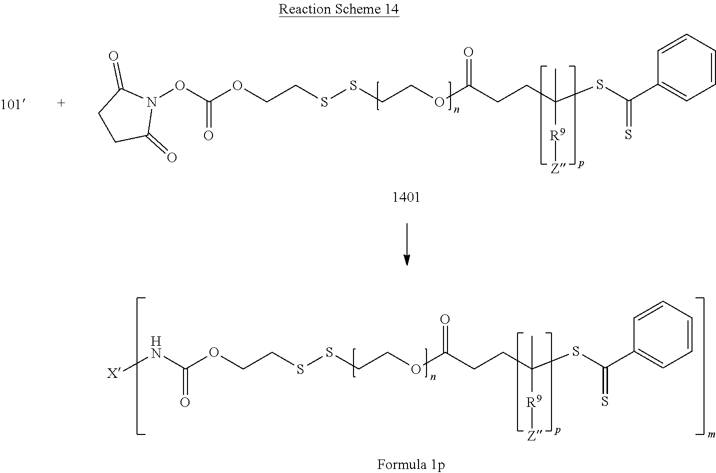

As illustrated in Reaction Scheme 14, an antigen, antibody, antibody fragment or ligand having native free surface amino group(s) (Formula 101′) is contacted with an excess (corresponding to the value of m) of a pyridyl di-thiol-poly(ethylene glycol)-NHS ester of Formula 1401 to yield the corresponding product according to Formula 1p.

The compositions corresponding to Formula 1p can be named as follows:

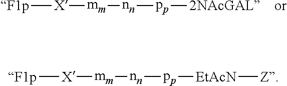

### Preparation of Fusion Proteins

Fusion protein compositions of Formula 2 can be expressed via art-accepted methodology using commercially available mammalian, bacterial, yeast, or insect cell expression vectors, and published, discovered, or engineered gene sequences. Sequences encoding X, Y and Z together with tag sequences can be cloned into an expression vector, for example, into the mammalian expression vector pSecTag A, where the fusion protein is inserted C-terminal to the Ig κ-chain secretion leader sequence. Various other cloning techniques can be employed, including site-directed mutagenesis and variations of the QuikChange protocol (Geiser, et al.), and are known by those skilled in the art. Fusion proteins can be transiently expressed in mammalian cells [e.g., in human embryonic kidney (HEK293) cells or Chinese hamster ovary (CHO) cells] by transient transfection with the above-described vectors using polyethylenimine. Transfected cells are cultured in a suitable medium (e.g., FreeStyle 293 medium, Life Technologies) supplemented, for example, with valproic acid or DMSO for about 7 days, after which the cells are removed by centrifugation and the culture supernatants are collected and sterilized by filtration.

Alternatively, the fusion proteins can be stably expressed by creating stably transfected mammalian cell lines. Additionally, expression vectors can be used to produce fusion proteins in bacteria, such as Escherichia coli, Corynebacterium, or Pseudomonas fluorescens by using compatible media (e.g LB, 2XYT, SOB, SOC, TB and other broths), supplements (e.g. glycerol, glucose, and other supplements), and appropriate growth and expression conditions. Expression systems in yeast can commonly use Saccharomyces cerevisiae and Pichia pastoris, or other organisms for the genera Saccharomyces, Pichia, Kluyveromyces, and Yarrowia, and less commonly used organisms like the filamentous fungi Aspergillus, Trichoderma, or Myceliophthora thermophila C1. Insect expression systems can utilize baculovirus infected insect cells or non-lytic insect cell expression to achieve protein expression levels in high quantity; most common insect cells include but are not limited to Sf9 and Sf21 (from Spodoptera frugiperda), Hi-5 (from Trichoplusia ni), and Schneider 2 and 3 (from Drosophila melanogaster).

The expressed fusion protein products can be purified from the culture supernatants by affinity chromatography (e.g., using a HisTrap Ni²⁺ sepharose column, GE Healthcare, for a His-tagged fusion protein), followed by other chromatographic polishing steps such as size exclusion chromatography (e.g., using a Superdex 75 column, GE Healthcare) or ion exchange chromatography. Protein purity can be verified, e.g., by Coomassie Brilliant Blue staining of SDS-PAGE gels and western blotting (e.g. anti-6×His tag western blotting for a His-tagged fusion protein). Protein concentration can be determined using the Beer-Lambert Law, for which the absorbance at 280 nm can be measured, e.g., using a NanoDrop 2000 (Thermo Scientific). The molecular weight and extinction coefficient can be estimated from the protein's amino acid sequence, e.g., using the ExPASy ProtParam tool. Endotoxin levels can be measured, e.g., using the HEK-Blue TLR⁴ reporter cell line (Invivogen) according to manufacturer's instructions.

### Preparation of Desialylated Antigens, Antibodies, Antibody Fragments and Ligands

Desialylated proteins can be produced via art-accepted methodology using commercially available neuraminidase (also known as acetyl-neuraminyl hydrolase or sialidase) enzyme or sulfuric acid. For enzymatic desialylation of a protein of interest, the protein can be incubated together with neuraminidase at 37° C. for 1 hour, or longer as necessary. For chemical desialylation through acid hydrolysis, a protein of interest can be treated with 0.025 N sulfuric acid at 80° C. for 1 hour, or longer as necessary. The desialylated protein can then be purified from the reaction mixture by immobilized metal ion affinity chromatography (e.g., using a HisTrap Ni2+ sepharose column, GE Healthcare), followed by size exclusion chromatography (e.g., using a Superdex 75 column, GE Healthcare). Protein purity can be verified, e.g., by Coomassie Brilliant Blue staining of SDS-PAGE gels and anti-6×His tag western blotting. Desialylation can be verified, e.g. by lectin-based detection of protein sialic acid content in western blots or colorimetric quantification of sialic acid content using commercially available kits (e.g. Abcam, ProZyme, or Sigma). Desialylated protein concentration can be determined using the Beer-Lambert Law, for which the absorbance at 280 nm can be measured, e.g., using a NanoDrop 2000 (Thermo Scientific). The molecular weight and extinction coefficient can be estimated from the protein's amino acid sequence, e.g., using the ExPASy ProtParam tool. Endotoxin levels can be measured, e.g., using the HEK-Blue TLR⁴ reporter cell line (Invivogen) according to manufacturer's instructions.

### Particular Processes and Last Steps

A compound of Formula 103′ is contacted with an excess (corresponding to the value of m) of a compound of Formula 106 to give the corresponding product of Formula 1a.

A compound of Formula 103′ is contacted with an excess (corresponding to the value of m) of a compound of Formula 201 to give the corresponding product of Formula 1b.

A compound of Formula 802, 902 or 1002 is contacted with an excess (corresponding to the value of m) of a compound of Formula 803 to give the corresponding product of Formula 1h, Formula 1i or Formula 1k, respectively.

A compound of Formula 1109 is contacted with an excess (corresponding to the value of m) of a compound of Formula 1107 to give the corresponding product of Formula 1m, particularly where n is about 80, p is about 30, q is about 4, and m being a function of the antigen is about 2 to 10.

A compound of Formula 1202 is contacted with an excess (corresponding to the value of m) of a compound of Formula 1107 to give the corresponding product of Formula 1n, particularly where n is about 1, p is about 30, q is about 4, and m being a function of the antigen is about 2 to 10.

### Particular Compositions

By way of non-limiting example, a particular group preferred for the compositions, pharmaceutical formulations, methods of manufacture and use of the present disclosure are the following combinations and permutations of substituent groups of Formula 1 (sub-grouped, respectively, in increasing order of preference):
X is a foreign transplant antigen against which transplant recipients develop an unwanted immune response, a foreign antigen to which patients develop an unwanted immune response, a therapeutic protein to which patients develop an unwanted immune response, a self-antigen to which patients develop an unwanted immune response, or a tolerogenic portion thereof.
X is a therapeutic protein to which patients develop an unwanted immune response selected from: Abatacept, Abciximab, Adalimumab, Adenosine deaminase, Ado-trastuzumab emtansine, Agalsidase alfa, Agalsidase beta, Aldeslukin, Alglucerase, Alglucosidase alfa, α-1-proteinase inhibitor, Anakinra, Anistreplase (anisoylated plasminogen streptokinase activator complex), Antithrombin III, Antithymocyte globulin, Ateplase, Bevacizumab, Bivalirudin, Botulinum toxin type A, Botulinum toxin type B, C1-esterase inhibitor, Canakinumab, Carboxypeptidase G2 (Glucarpidase and Voraxaze), Certolizumab pegol, Cetuximab, Collagenase, Crotalidae immune Fab, Darbepoetin-α, Denosumab, Digoxin immune Fab, Dornase alfa, Eculizumab, Etanercept, Factor VIIa, Factor VIII, Factor IX, Factor XI, Factor XIII, Fibrinogen, Filgrastim, Galsulfase, Golimumab, Histrelin acetate, Hyaluronidase, Idursulphase, Imiglucerase, Infliximab, Insulin (including rHu insulin and bovine insulin), Interferon-α2a, Interferon-α2b, Interferon-β1a, Interferon-β1b, Interferon-γ1b, Ipilimumab, L-arginase, L-asparaginase, L-methionase, Lactase, Laronidase, Lepirudin/hirudin, Mecasermin, Mecasermin rinfabate, Methoxy Ofatumumab, Natalizumab, Octreotide, Oprelvekin, Pancreatic amylase, Pancreatic lipase, Papain, Peg-asparaginase, Peg-doxorubicin HCl, PEG-epoetin-β, Pegfilgrastim, Peg-Interferon-α2a, Peg-Interferon-α2b, Pegloticase, Pegvisomant, Phenylalanine ammonia-lyase (PAL), Protein C, Rasburicase (uricase), Sacrosidase, Salmon calcitonin, Sargramostim, Streptokinase, Tenecteplase, Teriparatide, Tocilizumab (atlizumab), Trastuzumab, Type 1 alpha-interferon, Ustekinumab, and vW factor.
Especially where X is Abciximab, Adalimumab, Agalsidase alfa, Agalsidase beta, Aldeslukin, Alglucosidase alfa, Factor VIII, Factor IX, Infliximab, L-asparaginase, Laronidase, Natalizumab, Octreotide, Phenylalanine ammonia-lyase (PAL), or Rasburicase (uricase).
   Particularly where X is Factor VIII, Factor IX, uricase, PAL or asparaginase.
X is a self-antigen polypeptide selected for treating type 1 diabetes mellitus, pediatric multiple sclerosis, juvenile rheumatoid arthritis, celiac disease, or alopecia universalis.
   Especially where X is a self-antigen polypeptide selected for treating new onset type 1 diabetes mellitus, pediatric multiple sclerosis or celiac disease.
X is a foreign antigen to which patients develop an unwanted immune response
   From peanut, including conarachin (Ara h 1)
   From wheat, including Alpha-gliadin “33-mer” native (SEQ ID NO:24), Alpha-gliadin “33-mer” deamidated (SEQ ID NO:25), Alpha-gliadin (SEQ ID NO:26) and Omega-gliadin (SEQ ID NO: 27).
   From cat, including Fel d 1A (UNIPROT P30438) and Cat albumin (UNIPROT P49064).
   From dog, including Can f 1 (UNIPROT 018873) and Dog albumin (UNIPROT P49822).
X is a foreign transplant antigen against which transplant recipients develop an unwanted immune response, e.g. a human leukocyte antigen protein.
X is an antibody, antibody fragment or ligand that specifically binds a circulating protein or peptide or antibody, which circulating protein or peptide or antibody gives rise to transplant rejection, immune response against a therapeutic agent, autoimmune disease, and/or allergy.
   Especially where X binds an endogenous circulating protein or peptide or antibody.
Y is a linker selected from: Formula Ya, Formula Yb, Formula Yh, Formula Yi, Formula Yk, Formula Ym, Formula Yn, Formula Yo and Formula Yp.
   Especially where n is 8 to 90±10%, p is 20 to 100±10%, and q is 3 to 20±3.
      Particularly where n is 40 to 80±10%, p is 30 to 40±10%, and q is 4 to 12±3.
   Especially where Y is Formula Ya, Formula Yb, Formula Ym or Formula Yn.
      Particularly where n is 8 to 90±10%, p is 20 to 100±10% and q is 3 to 20±3.
         More particularly where n is 40 to 80±10%, p is 30 to 40±10%, and q is 4 to 12±3.
      Particularly where Z is conjugated to Y via an ethylacetamido group.
         More particularly where Z is conjugated to Y at its C1.
         More particularly where R⁸ is CMP.
         More particularly where R⁸ is CMP.
      Particularly where R⁸ is CMP.
Z is galactose, galactosamine or N-acetylgalactosamine.
   Especially where Z is galactose or N-acetylgalactosamine conjugated at C1, C2 or C6.
      Particularly where Z is galactose or N-acetylgalactosamine conjugated at C1 or C2.
         More particularly where Z is N-acetylgalactosamine conjugated at C1.

Each of the above-described groups and sub-groups are individually preferred and can be combined to describe further preferred aspects of the disclosure, for example but not by way of limitation, as follows:
X is a self-antigen polypeptide selected for treating type 1 diabetes mellitus, pediatric multiple sclerosis, juvenile rheumatoid arthritis, celiac disease, or alopecia universalis.
   Especially where X is a self-antigen polypeptide selected for treating new onset type 1 diabetes mellitus, pediatric multiple sclerosis or celiac disease.
      Particularly where Y is a linker selected from: Formula Ya, Formula Yb, Formula Yh, Formula Yi, Formula Yk, Formula Ym, Formula Yn, Formula Yo and Formula Yp.
         Especially where n is 8 to 90±10%, p is 20 to 100±10%, and q is 3 to 20±3.
         Particularly where n is 40 to 80±10%, p is 30 to 40±10%, and q is 4 to 12±3.
         Especially where Y is Formula Ya, Formula Yb, Formula Ym or Formula Yn.
         Particularly where n is 8 to 90±10%, p is 20 to 100±10% and q is 3 to 20±3.
         More particularly where n is 40 to 80±10%, p is 30 to 40±10%, and q is 4 to 12±3.
         Even more particularly where Z is conjugated to Y via an ethylacetamido group.
         More particularly where Z is conjugated to Y via an ethylacetamido group.
         Particularly where Z is conjugated to Y via an ethylacetamido group.
         Especially where Z is galactose, galactosamine or N-acetylgalactosamine.
         Particularly where Z is galactose or N-acetylgalactosamine conjugated at C1, C2 or C6.
         More particularly where Z is galactose or N-acetylgalactosamine conjugated at C1 or C2.
         Even more particularly where Z is N-acetylgalactosamine conjugated at C1.
      Particularly where Z is galactose, galactosamine or N-acetylgalactosamine.
         Especially where Z is galactose or N-acetylgalactosamine conjugated at C1, C2 or C6.
         Particularly where Z is galactose or N-acetylgalactosamine conjugated at C1 or C2.
         More particularly where Z is N-acetylgalactosamine conjugated at C1.
   Especially where Y is a linker selected from: Formula Ya, Formula Yb, Formula Yh, Formula Yi, Formula Yk, Formula Ym, Formula Yn, Formula Yo and Formula Yp.
      Particularly where n is 8 to 90±10%, p is 20 to 100±10%, and q is 3 to 20±3.
         More particularly where n is 40 to 80±10%, p is 30 to 40±10%, and q is 4 to 12±3.
      Particularly where Y is Formula Ya, Formula Yb, Formula Ym or Formula Yn.
         More particularly where n is 8 to 90±10%, p is 20 to 100±10% and q is 3 to 20±3.
         More preferably where n is 40 to 80±10%, p is 30 to 40±10%, and q is 4 to 12±3.
         More particularly where Z is conjugated to Y via an ethylacetamido group.
      Especially where Z is galactose, galactosamine or N-acetylgalactosamine.
         Particularly where Z is galactose or N-acetylgalactosamine conjugated at C1, C2 or C6.
         More particularly where Z is galactose or N-acetylgalactosamine conjugated at C1 or C2.
         More preferably where Z is N-acetylgalactosamine conjugated at C1.
m is an integer from about 1 to 100.
   mis 1, 2, 3, 4, 5, 6, 7, 8, 9, 10, 11, 12, 13, 14, 15, 16, 17, 18, 19, 20, 25, 30, 35, 40, 45, 50, 55, 60, 65 70, 75, 80, 85, 90, 95, 100 or 110.
   Particularly m is from about 1 to 20.
      m is 1, 2, 3, 4, 5, 6, 7, 8, 9, 10, 11, 12, 13, 14, 15, 16, 17, 18, 19, 20, 21 or 22.
      More particularly m is about 10.
         m is 9, 10 or 11.
n is an integer representing a mixture including from about 1 to 100
   n is 1, 2, 3, 4, 5, 6, 7, 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95, 99, 100, 105 or 110.
      Particularly n is about 8 to 90.
      Particularly n is 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95 or 99.
         More particularly n is about 40 to 80.
         More particularly n is 37, 40, 41, 45, 50, 54, 55, 59, 60, 65, 70, 75, 80, 82, 83 or 88.
   n represents a mixture encompassing the ranges 1-4, 2-4, 2-6, 3-8, 7-13, 6-14, 15-25, 26-30, 42-50, 46-57, 60-82, 85-90, 90-110 and 107-113.
      Particularly n represents a mixture encompassing the ranges 7-13, 6-14, 15-25, 26-30, 42-50, 46-57, 60-82, 85-90 and 82-99.
         More particularly n represents a mixture encompassing the ranges 36-44, 42-50, 46-57, 60-82 and 75-85.
   p is an integer representing a mixture including from about 2 to 150.
      p is 2, 3, 4, 5, 6, 7, 8, 9, 10, 11, 12, 13, 14, 15, 16, 17, 18, 19, 20, 25, 30, 35, 40, 45, 50, 55, 60, 65 70, 75, 80, 85, 90, 95, 100, 110, 120, 130, 140, 150, 160 or 165.
      Particularly where n is an integer representing a mixture including from about 1 to 100.
         Particularly n is 1, 2, 3, 4, 5, 6, 7, 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95, 99, 100, 105 or 110.
         More particularly where n is about 8 to 90.
         More particularly n is 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95 or 99.
         Even more particularly where n is about 40 to 80.
         Even more particularly n is 37, 40, 41, 45, 50, 54, 55, 59, 60, 65, 70, 75, 80, 82, 83 or 88.
         More particularly p is 18, 19, 20, 25, 30, 35, 40, 45, 50, 55, 60, 65 70, 75, 80, 85, 90, 95, 100 or 110.
         Particularly where n is an integer representing a mixture including from about 1 to 100.
         Particularly n is 1, 2, 3, 4, 5, 6, 7, 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95, 99, 100, 105 or 110.
         More particularly where n is about 8 to 90.
         More particularly n is 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85,
         Even more particularly where n is about 40 to 80.
         Even more particularly n is 37, 40, 41, 45, 50, 54, 55, 59, 60, 65, 70, 75, 80, 82, 83 or 88.
         More particularly p is 27, 28, 29, 30, 31, 32, 33, 34, 35, 36, 37, 38, 39, 40, 41, 42, 43, or 44.
         Particularly where n is 1, 2, 3, 4, 5, 6, 7, 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95, 99, 100, 105 or 110.
         More particularly where n is about 8 to 90.
         More particularly n is 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95 or 99.
         Even more particularly where n is about 40 to 80.
         Even more particularly n is 37, 40, 41, 45, 50, 54, 55, 59, 60, 65, 70, 75, 80, 82, 83 or 88.
   q is an integer representing a mixture including from about 1 to 44.
      q is 1, 2, 3, 4, 5, 6, 7, 8, 9, 10, 11, 12, 13, 14, 15, 16, 17, 18, 19, 20, 25, 30, 35, 40, 44 or 48.

By way of non-limiting example, a particular group preferred for the compositions, pharmaceutical formulations, methods of manufacture and use of the present disclosure are the following combinations and permutations of substituent groups of Formula 2 (sub-grouped, respectively, in increasing order of preference):
X is a therapeutic protein to which patients develop an unwanted immune response selected from: Abatacept, Abciximab, Adalimumab, Adenosine deaminase, Ado-trastuzumab emtansine, Agalsidase alfa, Agalsidase beta, Aldeslukin, Alglucerase, Alglucosidase alfa, α-1-proteinase inhibitor, Anakinra, Anistreplase (anisoylated plasminogen streptokinase activator complex), Antithrombin III, Antithymocyte globulin, Ateplase, Bevacizumab, Bivalirudin, Botulinum toxin type A, Botulinum toxin type B, C1-esterase inhibitor, Canakinumab, Carboxypeptidase G2 (Glucarpidase and Voraxaze), Certolizumab pegol, Cetuximab, Collagenase, Crotalidae immune Fab, Darbepoetin-α, Denosumab, Digoxin immune Fab, Dornase alfa, Eculizumab, Etanercept, Factor VIIa, Factor VIII, Factor IX, Factor XI, Factor XIII, Fibrinogen, Filgrastim, Galsulfase, Golimumab, Histrelin acetate, Hyaluronidase, Idursulphase, Imiglucerase, Infliximab, Insulin, Interferon-α2a, Interferon-α2b, Interferon-β1a, Interferon-β1b, Interferon-γ1b, Ipilimumab, L-arginase, L-asparaginase, L-methionase, Lactase, Laronidase, Lepirudin/hirudin, Mecasermin, Mecasermin rinfabate, Methoxy Ofatumumab, Natalizumab, Octreotide, Oprelvekin, Pancreatic amylase, Pancreatic lipase, Papain, Peg-asparaginase, Peg-doxorubicin HCl, PEG-epoetin-β, Pegfilgrastim, Peg-Interferon-α2a, Peg-Interferon-α2b, Pegloticase, Pegvisomant, Phenylalanine ammonia-lyase (PAL), Protein C, Rasburicase (uricase), Sacrosidase, Salmon calcitonin, Sargramostim, Streptokinase, Tenecteplase, Teriparatide, Tocilizumab (atlizumab), Trastuzumab, Type 1 alpha-interferon, Ustekinumab, and vW factor; provided that interferon (interferon alpha 2, interferon alpha 5, interferon alpha 6, or consensus interferon), Ribavirin, Nexavar/Sorafenib, Erbitus/Cetuximab, Avastatin/bevacizumab, and Herceptin/trastuzumab are excluded from the scope of Formula 2 when m′+m″ equals 1 and Z is DOM 26h-196-61 or another of the liver-targeting molecules described in US 2013/0078216.
   Especially where X is Abciximab, Adalimumab, Agalsidase alfa, Agalsidase beta, Aldeslukin, Alglucosidase alfa, Factor VIII, Factor IX, Infliximab, L-asparaginase, Laronidase, Natalizumab, Octreotide, Phenylalanine ammonia-lyase (PAL), or Rasburicase (uricase).
      Particularly where X is Factor VIII, Factor IX, uricase, PAL or asparaginase.
X is a self-antigen polypeptide selected for treating type 1 diabetes mellitus, pediatric multiple sclerosis, juvenile rheumatoid arthritis, celiac disease, or alopecia universalis.
   Especially where X is a self-antigen polypeptide selected for treating new onset type 1 diabetes mellitus, pediatric multiple sclerosis or celiac disease.
X is a foreign antigen to which patients develop an unwanted immune response
   From peanut, including conarachin (Ara h 1).
   From wheat, including Alpha-gliadin “33-mer” native (SEQ ID NO:24), Alpha-gliadin “33-mer” deamidated (SEQ ID NO:25), Alpha-gliadin (SEQ ID NO:26) and Omega-gliadin (SEQ ID NO: 27).
   From cat, including Fel d 1A (UNIPROT P30438) and Cat albumin (UNIPROT P49064).
   From dog, including Can f 1 (UNIPROT 018873) and Dog albumin (UNIPROT P49822).
X s is a foreign transplant antigen against which transplant recipients develop an unwanted immune response, e.g. a human leukocyte antigen protein.
X is an antibody, antibody fragment or ligand that specifically binds a circulating protein or peptide or antibody, which circulating protein or peptide or antibody gives rise to transplant rejection, immune response against a therapeutic agent, autoimmune disease, and/or allergy.
   Especially where X binds an endogenous circulating protein or peptide or antibody.
Y is Gly₃Ser.
Z is Anti-ASGPR Dom26h-196-61 or a conservative substitution.
m′+m″ equals 1 or 2 and X is a full length protein, including protein therapeutics.
m′+m″ equals 1 or 2 where m′ is 1 and m″ is 0 or 1.
m′+m″ equals 2 to about 10 where X is a group of self-antigen peptides (epitopes) known to be associated with a particular autoimmune disease or for treatment of genetically diverse target populations.
   m′+m″ equals about 4 to 7 where the autoimmune disease is multiple sclerosis and X is independently selected from: MBP13-32 (SEQ ID NO:11), MBP83-99 (SEQ ID NO:12), MBP111-129 (SEQ ID NO:13), MBP146-170 (SEQ ID NO:14), MOG1-20 (SEQ ID NO:15), MOG35-55 (SEQ ID NO:16) and PLP139-154 (SEQ ID NO:17).
      “m′+m” equals 7 and X is, respectively, MBP13-32 (SEQ ID NO:11), MBP83-99 (SEQ ID NO: 12), MBP111-129 (SEQ ID NO:13), MBP146-170 (SEQ ID NO:14), MOG1-20 (SEQ ID NO:15), MOG35-55 (SEQ ID NO:16) and PLP139-154 (SEQ ID NO:17).

As with the above discussion regarding Formula 1, each of the above-described groups and sub-groups for Formula 2 are individually preferred and can be combined to describe further preferred aspects of the disclosure.

### Utility, Testing and Administration

### General Utility

The compositions of the disclosure find use in a variety of applications including, as will be appreciated by those in the art, treatment of transplant rejection, immune response against a therapeutic agent, autoimmune disease, and food allergy.

In a preferred embodiment, the compositions of the disclosure are used to modulate, particularly down-regulate, antigen-specific undesirable immune response.

The compositions of the disclosure are useful to bind and clear from the circulation specific undesired proteins, including antibodies endogenously generated in a patient (i.e., not exogenous antibodies administered to a patient), peptides and the like, which cause autoimmunity and associated pathologies, allergy, inflammatory immune responses, and anaphylaxis.

In the present disclosure, antigens are targeted to the liver for presentation via antigen-presenting cells to specifically down-regulate the immune system or for clearance of unwanted circulating proteins. This is distinct from previous uses of liver targeting, for example as described in US 2013/0078216, where the purpose of liver-targeting molecules such as DOM26h-196-61 was the delivery of therapeutic agents to treat liver diseases such as fibrosis, hepatitis, Cirrhosis and liver cancer.

The present disclosure provides compositions and methods to treat unwanted immune response to self-antigens and foreign antigens, including but not limited to: a foreign transplant antigen against which transplant recipients develop an unwanted immune response (e.g., transplant rejection), a foreign antigen to which patients develop an unwanted immune (e.g., allergic or hypersensitivity) response, a therapeutic agent to which patients develop an unwanted immune response (e.g., hypersensitivity and/or reduced therapeutic activity), a self antigen to which patients develop an unwanted immune response (e.g., autoimmune disease)

Autoimmune disease states that can be treated using the methods and compositions provided herein include, but are not limited to: Acute Disseminated Encephalomyelitis (ADEM); Acute interstitial allergic nephritis (drug allergies); Acute necrotizing hemorrhagic leukoencephalitis; Addison's Disease; Alopecia areata; Alopecia universalis; Ankylosing Spondylitis; Arthritis, juvenile; Arthritis, psoriatic; Arthritis, rheumatoid; Atopic Dermatitis; Autoimmune aplastic anemia; Autoimmune gastritis; Autoimmune hepatitis; Autoimmune hypophysitis; Autoimmune oophoritis; Autoimmune orchitis; Autoimmune polyendocrine syndrome type 1; Autoimmune polyendocrine syndrome type 2; Autoimmune thyroiditis; Behcet's disease; Bronchiolitis obliterans; Bullous pemphigoid; Celiac disease; Churg-Strauss syndrome; Chronic inflammatory demyelinating polyneuropathy; Cicatricial pemphigoid; Crohn's disease; Coxsackie myocarditis; Dermatitis herpetiformis Duhring; Diabetes mellitus (Type 1); Erythema nodosum; Epidermolysis bullosa acquisita, Giant cell arteritis (temporal arteritis); Giant cell myocarditis; Goodpasture's syndrome; Graves' disease; Guillain-Barre syndrome; Hashimoto's encephalitis; Hashimoto's thyroiditis; IgG4-related sclerosing disease; Lambert-Eaton syndrome; Mixed connective tissue disease; Mucha-Habermann disease; Multiple sclerosis; Myasthenia gravis; Optic neuritis; Neuromyelitis optica; Pemphigus vulgaris and variants; Pernicious angemis; Pituitary autoimmune disease; Polymyositis; Postpericardiotomy syndrome; Premature ovarian failure; Primary Biliary Cirrhosis; Primary sclerosing cholangitis; Psoriasis; Rheumatic heart disease; Sjogren's syndrome; Systemic lupus erythematosus; Systemic sclerosis; Ulcerative colitis; Undifferentiated connective tissue disease (UCTD); Uveitis; Vitiligo; and Wegener's granulomatosis.

A particular group of autoimmune disease states that can be treated using the methods and compositions provided herein include, but are not limited to: Acute necrotizing hemorrhagic leukoencephalitis; Addison's Disease; Arthritis, psoriatic; Arthritis, rheumatoid; Autoimmune aplastic anemia; Autoimmune hypophysitis; Autoimmune gastritis; Autoimmune polyendocrine syndrome type 1; Bullous pemphigoid; Celiac disease; Coxsackie myocarditis; Dermatitis herpetiformis Duhring; Diabetes mellitus (Type 1); Epidermolysis bullosa acquisita; Giant cell myocarditis; Goodpasture's syndrome; Graves' disease; Hashimoto's thyroiditis; Mixed connective tissue disease; Multiple sclerosis; Myasthenia gravis; Neuromyelitis optica; Pernicious angemis; Pemphigus vulgaris and variants; Pituitary autoimmune disease; Premature ovarian failure; Rheumatic heart disease; Systemic sclerosis; Sjogren's syndrome; Systemic lupus erythematosus; and Vitiligo.

In the embodiments employing an antigen against which an unwanted immune response is developed, such as food antigens, treatment can be provided for reactions against, for example: peanut, apple, milk, egg whites, egg yolks, mustard, celery, shrimp, wheat (and other cereals), strawberry and banana.

As will be appreciated by those skilled in the art, a patient can be tested to identify a foreign antigen against which an unwanted immune response has developed, and a composition of the disclosure can be developed based on that antigen.

### Testing

In establishing the utility of the compositions and methods of the disclosure, specificity in binding to antigen-presenting cells in the liver (particularly binding to hepatocytes and specifically ASGPR) should initially be determined. This can be accomplished, for example, by employing a marker (such as the fluorescent marker phycoerythrin (“PE”)) in a composition of the disclosure. The composition is administered to suitable experimental subjects. Controls, e.g., unconjugated PE or vehicle (saline) are administered to other group(s) of subjects. The composition and controls are allowed to circulate for a period of 1 to 5 hours, after which the spleens and livers of the subjects are harvested and measured for fluorescence. The specific cells in which fluorescence is found can be subsequently identified. Compositions of the disclosure, when tested in this manner, show higher levels of concentration in the antigen-presenting cells of the liver as compared with unconjugated PE or vehicle.

Effectiveness in immune modulation can be tested by measuring the proliferation of OT-I CD8⁺ cells (transplanted into host mice) in response to the administration of a composition of the disclosure incorporating a known antigen, such as ovalbumin (“OVA”), as compared with administration of the antigen alone or just vehicle. Compositions of the disclosure, when tested in this manner, show an increase of OT1 cell proliferation as compared with antigen alone or vehicle, demonstrating increased CD8⁺ T-cell cross-priming. To distinguish T cells being expanded into a functional effector phenotype from those being expanded and deleted, the proliferating OT-I CD8⁺ T cells can be phenotypically analyzed for molecular signatures of exhaustion [such as programmed death-1 (PD-1), FasL, and others], as well as annexin-V as a hallmark of apoptosis and thus deletion. The OT-I CD8⁺ T cells can also be assessed for their responsiveness to an antigen challenge with adjuvant in order to demonstrate functional non-responsiveness, and thus immune tolerance, towards the antigen. To do so, the cells are analyzed for inflammatory signatures after administration of compositions of the disclosure into host mice followed by an antigen challenge. Compositions of the disclosure when tested in this manner demonstrate very low (e.g., background) levels of inflammatory OT-I CD8⁺ T cell responses towards OVA, thus demonstrating immune tolerance.

Humoral immune response can be tested by administering a composition of the disclosure incorporating a known antigen, such as OVA, as compared with the administration of the antigen alone or just vehicle, and measuring the levels of resulting antibodies. Compositions of the disclosure when tested in this manner show very low (e.g., background) levels of antibody formation responsive to their administration and the administration of vehicle, with significantly higher levels of antibody formation responsive to administration of the antigen.

Effectiveness in tolerization against an antigen can be tested as above with reference to humoral immune response, where several weeks following treatment(s) with a composition of the disclosure a group of subjects is challenged by administration of the antigen alone, followed by measuring the levels of antibodies to the antigen. Compositions of the disclosure when tested in this manner show low levels of antibody formation responsive to challenge with the antigen in groups pretreated with such compositions as compared to groups that are not pretreated.

Disease-focused experimental models are well known to those skilled in the art and include the NOD (or non-obese diabetic) mouse model of autoimmunity and tolerance and the EAE (experimental autoimmune encephalomyelitis) model for the human inflammatory demyelinating disease, multiple sclerosis. In particular, the NOD mouse develops spontaneous autoimmune diabetes (similar to type 1a diabetes in humans). Groups of NOD mice are treated with test compound or a negative control, followed by measurement of BLOOD GLUCOSE. Successful treatment corresponds to likelihood of treating diabetes in humans or proof of mechanism for approaches to the treatment of other autoimmune diseases. (See, e.g., Anderson and Bluestone, Annu. Rev. Immunol. 2005; 23:447-85.)

### Administration

The compositions of the disclosure are administered at a therapeutically effective dosage, e.g., a dosage sufficient to provide treatment for the disease states previously described. Administration of the compounds of the disclosure or the pharmaceutically acceptable salts thereof can be via any of the accepted modes of administration for agents that serve similar utilities.

While human dosage levels have yet to be optimized for the compounds of the disclosure, these can initially be extrapolated from the about 10 μg to 100 μg doses administered for mice. Generally, an individual human dose is from about 0.01 to 2.0 mg/kg of body weight, preferably about 0.1 to 1.5 mg/kg of body weight, and most preferably about 0.3 to 1.0 mg/kg of body weight. Treatment can be administered for a single day or a period of days, and can be repeated at intervals of several days, one or several weeks, or one or several months. Administration can be as a single dose (e.g., as a bolus) or as an initial bolus followed by continuous infusion of the remaining portion of a complete dose over time, e.g., 1 to 7 days. The amount of active compound administered will, of course, be dependent on any or all of the following: the subject and disease state being treated, the severity of the affliction, the manner and schedule of administration and the judgment of the prescribing physician. It will also be appreciated that amounts administered will depend upon the molecular weight of the antigen, antibody, antibody fragment or ligand as well as the size of the linker.

The compositions of the disclosure can be administered either alone or in combination with other pharmaceutically acceptable excipients. While all typical routes of administration are contemplated, it is presently preferred to provide liquid dosage forms suitable for injection. The formulations will typically include a conventional pharmaceutical carrier or excipient and a composition of the disclosure or a pharmaceutically acceptable salt thereof. In addition, these compositions can include other medicinal agents, pharmaceutical agents, carriers, and the like, including, but not limited to the therapeutic protein, peptide, antibody or antibody-like molecule corresponding to the antigen (X) employed in the composition of the disclosure, and other active agents that can act as immune-modulating agents and more specifically can have inhibitory effects on B-cells, including anti-folates, immune suppressants, cytostatics, mitotic inhibitors, and anti-metabolites, or combinations thereof.

Generally, depending on the intended mode of administration, the pharmaceutically acceptable composition will contain about 0.1% to 95%, preferably about 0.5% to 50%, by weight of a composition of the disclosure, the remainder being suitable pharmaceutical excipients, carriers, etc. Dosage forms or compositions containing active ingredient in the range of 0.005% to 95% with the balance made up from non-toxic carrier can be prepared.

Liquid pharmaceutically administrable compositions can, for example, be prepared by dissolving, dispersing, etc. an active composition of the disclosure (e.g., a lyophilized powder) and optional pharmaceutical adjuvants in a carrier, such as, for example, water (water for injection), saline, aqueous dextrose, glycerol, glycols, ethanol or the like (excluding galactoses), to thereby form a solution or suspension. If desired, the pharmaceutical composition to be administered can also contain minor amounts of nontoxic auxiliary substances such as wetting agents, emulsifying agents, stabilizing agents, solubilizing agents, pH buffering agents and the like, for example, sodium acetate, sodium citrate, cyclodextrine derivatives, sorbitan monolaurate, triethanolamine acetate and triethanolamine oleate, etc., osmolytes, amino acids, sugars and carbohydrates, proteins and polymers, salts, surfactants, chelators and antioxidants, preservatives, and specific ligands. Actual methods of preparing such dosage forms are known, or will be apparent, to those skilled in this art; for example, see Remington: The Science and Practice of Pharmacy, Pharmaceutical Press, 22nd Edition, 2012. The composition or formulation to be administered will, in any event, contain a quantity of the active compound in an amount effective to treat the symptoms of the subject being treated.

### EXAMPLES

The following examples serve to more fully describe the manner of using the above-described disclosure, as well as to set forth the best modes contemplated for carrying out various aspects of the disclosure. It is understood that these examples in no way serve to limit the true scope of this disclosure, but rather are presented for illustrative purposes. All references cited herein are incorporated by reference in their entirety.

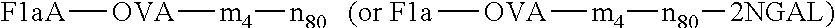

### Example 1

1A. Formula 103′ where X′ is OVA and m is 4

In an endotoxin-free tube, OVA (5.0 mg, 0.00012 mmol) was added to 100 μl of pH 8.0 PBS containing 5 mM EDTA and stirred. Separately, 1 mg of Traut's Reagent was dissolved in 100 μl of pH 7.0 PBS, and 16 μl (0.00119 mmol) of the Traut's Reagent solution so obtained was added to the stirred solution of OVA with continued stirring. After 1 hour, excess Traut's Reagent was removed using a centrifugal size exclusion column to afford the corresponding product of Formula 103′.

1B. Formula 106A where n is 80

In an endotoxin-free tube, galactosamine (10.0 mg, 0.04638 mmol) was dissolved with stirring in 100 μl of pH 8.0 PBS containing 5 mM EDTA. Pyridyl dithiol-poly(ethylene glycol)-NHS ester (Formula 104 where n is 80) (16.23 mg, 0.00464 mmol) dissolved in 100 μl of pH 7.0 PBS was added to the stirring solution of galactosamine. After 1 hour, the resulting pyridyl dithiol-poly(ethylene glycol)-N-acetylgalactosamine (Formula 106A) was ready to be used without further purification.

1C. Formula 1aA where X′ is OVA, m is 4, n is 80 (and Z′ is C2 Galactosamine)

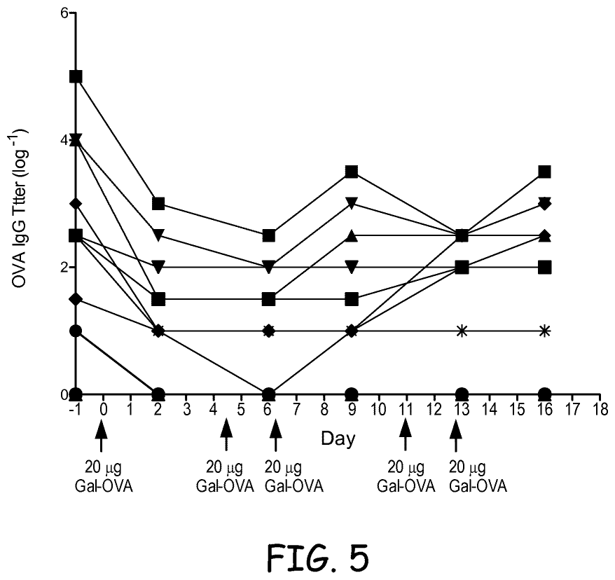

The purified OVA-Traut conjugate of Formula 103′ prepared in Example 1A was added directly to the stirring product of Formula 106A prepared in Example 1B. After 1 hour, the resulting product of Formula 1a was purified by passing the reaction mixture through a centrifugal size exclusion column. Characterization (UHPLC SEC, gel electrophoresis) confirmed product identity. (See FIG. 5.)

### 1D. Other Compounds of Formula 103′

By following the procedure described in Example 1A and substituting OVA with the following:
Abciximab,
Adalimumab,
Agalsidase alfa,
Agalsidase beta,
Aldeslukin,
Alglucosidase alfa,
Factor VIII,
Factor IX,
L-asparaginase,
Laronidase,
Octreotide,
Phenylalanine ammonia-lyase,
Rasburicase,
Insulin (SEQ ID NO:5),
GAD-65 (SEQ ID NO:6),
IGRP (SEQ ID NO:7)
MBP (SEQ ID NO:8),
MOG (SEQ ID NO:9),
PLP (SEQ ID NO:10),
MBP13-32 (SEQ ID NO:11),
MBP83-99 (SEQ ID NO:12),
MBP111-129 (SEQ ID NO:13),
MBP146-170 (SEQ ID NO:14),
MOG1-20 (SEQ ID NO:15),
MOG35-55 (SEQ ID NO:16),
PLP139-154 (SEQ ID NO:17),
MART1 (SEQ ID NO:18),
Tyrosinase (SEQ ID NO:19),
PMEL (SEQ ID NO:20),
Aquaporin-4 (SEQ ID NO:21),
S-arrestin (SEQ ID NO:22),
IRBP (SEQ ID NO:23),
Conarachin (UNIPROT Q6PSU6),
Alpha-gliadin “33-mer” native (SEQ ID NO:24),
Alpha-gliadin “33-mer” deamidated (SEQ ID NO:25),
Alpha-gliadin (SEQ ID NO:26),
Omega-gliadin (SEQ ID NO:27),
Fel d 1A (UNIPROT P30438),
Cat albumin (UNIPROT P49064),
Can f 1 (UNIPROT O18873),
Dog albumin (UNIPROT P49822), and
RhCE example (UNIPROT P18577), there are obtained the following corresponding compounds of Formula 103′ where:
X is Abciximab and m is 10,
X is Adalimumab and m is 11,
X is Agalsidase alfa and m is 14,
X is Agalsidase beta and m is 14,
X is Aldeslukin and m is 6,
X is Alglucosidase alfa and m is 13,
X is Factor VIII and m is 100,
X is Factor IX and m is 18,
X is L-asparaginase and m is 5,
X is Laronidase and m is 7,
X is Octreotide and m is 1,
X is Phenylalanine ammonia-lyase and m is 12,
X is Rasburicase and m is 12,
X is Insulin (SEQ ID NO:5) and m is 2,
X is GAD-65 (SEQ ID NO:6) and m is 8,
X is IGRP (SEQ ID NO:7) and m is 7,
X is MBP (SEQ ID NO:8) and m is 6,
X is MOG (SEQ ID NO:9) and m is 5,
X is PLP (SEQ ID NO:10) and m is 8,
X is MBP13-32 (SEQ ID NO:11) and m is 1,
X is MBP83-99 (SEQ ID NO:12) and m is 1,
X is MBP111-129 (SEQ ID NO:13) and m is 1,
X is MBP146-170 (SEQ ID NO:14) and m is 2,
X is MOG1-20 (SEQ ID NO:15) and m is 1,
X is MOG35-55 (SEQ ID NO:16) and m is 2,
X is PLP139-154 (SEQ ID NO:17) and m is 3,
X is MART1 (SEQ ID NO:18) and m is 4,
X is Tyrosinase (SEQ ID NO:19) and m is 8,
X is PMEL (SEQ ID NO:20) and m is 5,
X is Aquaporin-4 (SEQ ID NO:21) and m is 4,
X is S-arrestin (SEQ ID NO:22) and m is 12,
X is IRBP (SEQ ID NO:23) and m is 21,
X is Conarachin and m is 21,
X is Alpha-gliadin “33-mer” native (SEQ ID NO:24) and m is 1,
X is Alpha-gliadin “33-mer” deamidated (SEQ ID NO:25) and m is 1,
X is Alpha-gliadin (SEQ ID NO:26) and m is 1,
X is Omega-gliadin (SEQ ID NO:27) and m is 1,
X is Fel d 1 and m is 4,
X is Cat albumin and m is 16,
X is Can f 1 and m is 6,
X is Dog albumin and m is 23, and
X is RhCE example and m is 10.

### 1E. Other Compounds of Formula 1aA

By following the procedure described in Example 1C and substituting the compounds of Formula 103′, for example as obtained in Example 1D, there are obtained the following corresponding compounds of Formula 1aA:
F1aA-Abciximab-m₁₀-n₈₀,
F1aA-Adalimumab-m₁₁-n₈₀,
F1aA-Agalsidase alfa-m₁₄-80,
F1aA-Agalsidase beta-m₁₄-n₈₀,
F1aA-Aldeslukin-m₆-n₈₀,
F1aA-Alglucosidase alfa-m₁₃-n₈₀,
F1aA-Factor VIII-m₁₀₀-n₈₀,
F1aA-Factor IX-m₁₈-n₈₀,
F1aA-L-asparaginase-m₅-n₈₀,
F1aA-Laronidase-m₇-n₈₀,
F1aA-Octreotide-m₁-n₈₀,
F1aA-Phenylalanine ammonia-lyase-m₁₂-n₈₀,
F1aA-Rasburicase-m₁₂-n₈₀,
F1aA-Insulin-m₂-n₈₀,
F1aA-GAD-65-m₈-n₈₀,
F1aA-IGRP-m₇-n₈₀,
F1aA-MBP-m₆-n₈₀,
F1aA-MOG-m₅-n₈₀,
F1aA-PLP-m₈-n₈₀,
F1aA-MBP13-32-m₁-n₈₀,
F1aA-MBP83-99-m₁-n₈₀,
F1aA-MBP111-129-m₁-n₈₀,
F1aA-MBP146-170-m₂-n₈₀,
F1aA-MOG1-20-m₁-n₈₀,
F1aA-MOG35-55-m₂-n₈₀,
F1aA-PLP139-154-m₃-n₈₀,
F1aA-MART1-m₄-n₈₀,
F1aA-Tyrosinase-m₈-n₈₀,
F1aA-PMEL-m₅-n₈₀,
F1aA-Aquaporin-4-m₄-n₈₀,
F1aA-S-arrestin-m₁₂-n₈₀,
F1aA-IRBP-m₂₁-n₈₀,
F1aA-Conarachin-m₂₁-n₈₀,
F1aA-Alpha-gliadin “33-mer” native-m₁-n₈₀,
F1aA-Alpha-gliadin “33-mer” deamidated-m₁-n₈₀,
F1aA-Alpha-gliadin-m₁-n₈₀,
F1aA-Omega-gliadin-m₁-n₈₀,
F1aA-Fel d 1-m₄-n₈₀,
F1aA-Cat albumin-m₁₆-n₈₀,
F1aA-Can f 1-m₆-n₈₀,
F1aA-Dog albumin-m₂₃-n₈₀, and
F1aA-RhCE-m₁₀-n₈₀.

### 1F. Other Compounds of Formula 106A

By following the procedure described in Example 1B and substituting the pyridyl dithiol-poly(ethylene glycol)-NHS ester (Formula 104 where n is 80) with the following:
Formula 104 where n is 12,
Formula 104 where n is 33,
Formula 104 where n is 40,
Formula 104 where n is 43,
Formula 104 where n is 50,
Formula 104 where n is 60,
Formula 104 where n is 75, and
Formula 104 where n is 80, there are obtained the following corresponding compounds of Formula 106A where:
n is 12,
n is 33,
n is 40,
n is 43,
n is 50,
n is 60,
n is 75, and
n is 84,

### 1G. Other Compounds of Formula 1aA

By following the procedure described in Example 1E and substituting the compound of Formula 106A with the compounds obtained in Example 1F, there are obtained the corresponding compounds of Formula 1aA where n is 12, 33, 40, 43, 50, 60, 75 and 84, such as:
F1aA-Insulin-m₂-n₁₂,
F1aA-Insulin-m₂-n₃₃,
F1aA-Insulin-m₂-n₄₀,
F1aA-Insulin-m₂-n₄₃,
F1aA-Insulin-m₂-n₅₀,
F1aA-Insulin-m₂-n₆₀,
F1aA-Insulin-m₂-n₇₅, and
F1aA-Insulin-m₂-n₈₄.

### Example 2

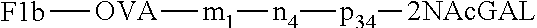

2A. Formula 103′ where X′ is Ovalbumin and m is 1

In an endotoxin-free tube, OVA (6.5 mg, 0.000155 mmol) was added to 200 μl of pH 8.0 PBS containing 5 mM EDTA and stirred. Separately, 1 mg of Taut's Reagent was dissolved in 100 μl of pH 7.0 PBS, and 43 μl (0.00310 mmol) of the Traut's Reagent solution so obtained was added to the stirred solution of OVA with continued stirring. After 1 hour, non-reacted Traut's Reagent was removed using a centrifugal size exclusion column to afford the product of Formula 103′.

2B. Formula 1b where X′ is Ovalbumin, m is 1, n is 4, p is 34, R⁹ is a Direct Bond and Z″ is 2NAcGAL

In a micro centrifuge tube, poly(Galactosamine Methacrylate)-(pyridyl disulfide) (Formula 201) (20.0 mg, 0.0020 mmol) was solubilized in 50 μl of pH 8.0 PBS containing 5 mM EDTA. To this was added the purified OVA-Traut product from Example 2A followed by stirring for 1 hour. The resulting product of Formula 1b was purified by passing the reaction mixture through a centrifugal size exclusion column. Characterization (UHPLC SEC, gel electrophoresis) confirmed the identity of the product. (See FIG. 5.)

### 2C. Other Compounds of Formula 1b

By following the procedure described in Example 2B and substituting the compounds of Formula 103′, for example as obtained in Example 1D, there are obtained the following corresponding compounds of Formula 1b:
F1b-Abciximab-m₁₀-n₄-p₃₄-2NAcGAL,
F1b-Adalimumab-m₁₁-n₄-p₃₄-2NAcGAL,
F1b-Agalsidase alfa-m₁₄-n₄-p₃₄-2NAcGAL,
F1b-Agalsidase beta-m₁₄-n₄-p₃₄-2NAcGAL,
F1b-Aldeslukin-m₆-n₄-p₃₄-2NAcGAL,
F1b-Alglucosidase alfa-m₁₃-n₄-p₃₄-2NAcGAL,
F1b-Factor VIII-m₁₀₀-n₄-p₃₄-2NAcGAL,
F1b-Factor IX-m₁₈-n₄-p₃₄-2NAcGAL,
F1b-L-asparaginase-m₅-n₄-p₃₄-2NAcGAL,
F1b-Laronidase-m₇-n₄-p₃₄-2NAcGAL,
F1b-Octreotide-m₁-n₄-p₃₄-2NAcGAL,
F1b-Phenylalanine ammonia-lyase-m₁₂-n₄-p₃₄-2NAcGAL,
F1b-Rasburicase-m₁₂-n₄-p₃₄-2NAcGAL,
F1b-Insulin-m₂-n₄-p₃₄-2NAcGAL,
F1b-GAD-65-m₈-n₄-p₃₄-2NAcGAL,
F1b-IGRP-m₇-n₄-p₃₄-2NAcGAL,
F1b-MBP-m₆-n₄-p₃₄-2NAcGAL,
F1b-MOG-m₅-n₄-p₃₄-2NAcGAL,
F1b-PLP-m₈-n₄-p₃₄-2NAcGAL,
F1b-MBP13-32-m₁-n₄-p₃₄-2NAcGAL,
F1b-MBP83-99-m₁-n₄-p₃₄-2NAcGAL,
F1b-MBP111-129-m₁-n₄-p₃₄-2NAcGAL,
F1b-MBP146-170-m₂-n₄-p₃₄-2NAcGAL,
F1b-MOG1-20-m₁-n₄-p₃₄-2NAcGAL,
F1b-MOG35-55-m₂-n₄-p₃₄-2NAcGAL,
F1b-PLP139-154-m₃-n₄-p₃₄-2NAcGAL,
F1b-MART1-m₄-n₄-p₃₄-2NAcGAL,
F1b-Tyrosinase-m₈-n₄-p₃₄-2NAcGAL,
F1b-PMEL-m₅-n₄-p₃₄-2NAcGAL,
F1b-Aquaporin-4-m₄-n₄-p₃₄-2NAcGAL,
F1b-S-arrestin-m₁₂-n₄-p₃₄-2NAcGAL,
F1b-IRBP-m₂₁-4-p₃₄-2NAcGAL,
F1b-Conarachin-m₂₁-n₄-p₃₄-2NAcGAL,
F1b-Alpha-gliadin “33-mer” native-m₁-n₄-p₃₄-2NAcGAL,
F1b-Alpha-gliadin “33-mer” deamidated-m₁-n₄-p₃₄-2NAcGAL,
F1b-Alpha-gliadin-m₁-n₄-p₃₄-2NAcGAL,
F1b-Omega-gliadin-m₁-n₄-p₃₄-2NAcGAL,
F1b-Fel d 1-m₄-n₄-p₃₄-2NAcGAL,
F1b-Cat albumin-m₁₆-n₄-p₃₄-2NAcGAL,

F1b-Can f 1-m₆-n₄-p₃₄-2NAcGAL,
F1b-Dog albumin-m₂₃-n₄-p₃₄-2NAcGAL, and
F1b-RhCE-m₁₀-n₄-p₃₄-2NAcGAL.

### Example 3

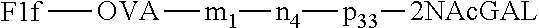

3A. Formula 1f where X′ is Ovalbumin and m is 1, n is 4, p is 33, R⁹ is a Direct Bond and Z″ is 2NAcGAL

In an endotoxin-free tube, OVA (4.0 mg, 0.0000952381 mmol) was added to 0.1 ml of pH 7.4 PBS and stirred. Separately, poly-(n-Acetylgalactosamine)-p-nitrophenyol carbonate of Formula 601 where n is 4 and p is 33 (33.0 mg, 0.002380952 mmol) was added to 100 μl of pH 7.5 PBS and vortexed until dissolved. The two solutions were combined and the mixture was stirred vigorously for 1 hour. The mixture was then collected and dialyzed for 3 days against pH 7.4 PBS (30 kDa molecular weight cut off) to afford the product of Formula 1f.

### Example 4

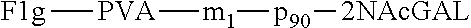

4A. Formula 1g where X′ is Ovalbumin and m is 1, p is 90. R⁹ is a Direct Bond and Z″ is 2NACGAL

In an endotoxin-free tube, OVA (5.0 mg, 0.000119048 mmol) was added to 0.2 ml of pH 7.4 PBS and stirred. To the stirring solution was added 75 mg (0.00297619 mmol) of Poly(Galactosamine Methacrylate)-NHS (Formula 701) dissolved in 0.4 ml of pH 7.4 PBS. The mixture was allowed to stir for 2 hours. The mixture was then collected and dialyzed for 3 days against pH 7.4 PBS (30 kDa molecular weight cut off) to afford the product of Formula 1g.

### Example 5

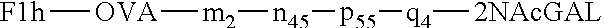

5A. Formula 802′ where X′ is Ovalbumin, m is 2 and n is 45

In an endotoxin-free tube, OVA (3.0 mg, 0.0000714286 mmol) was added to 150 μl of pH 8.0 PBS containing 5 mM EDTA and stirred. Dibenzocyclooctyne-PEG-(p-nitrophenyl carbonate) (Formula 801) (5.265 mg, 0.002142857 mmol) dissolved in DMF was added to the OVA solution and stirred for 1 hour. The excess dibenzocyclooctyne-PEG-(p-nitrophenyl carbonate) was removed using a centrifugal size exclusion column to afford the product of Formula 802′.

5B. Formula 1h where X′ is Ovalbumin, m is 2, n is 45, p is 55, q is 4, R⁸ is CH₂, R⁹ is a Direct Bond and Z″ is 2NAcGAL

Poly(Galactosamine Methacrylate)-N3 (Formula 803 where p is 55, q is 4 and Z″ is N-acetylgalactosamine) (33 mg, 0.002142857 mmol) was dissolved in 100 μl of pH 7.4 PBS and added to the product of Example 5A with stirring. After 1 hour, the resulting product of Formula 1h was purified by centrifugal size exclusion chromatography.

### Example 6

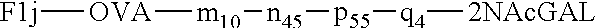

In an endotoxin-free tube, OVA (5.0 mg, 0.00019 mmol) was added to 150 μl of pH 8.0 PBS containing 5 mM EDTA and stirred. Separately, 1 mg of Taut's Reagent was dissolved in 100 μl of pH 7.0 PBS, and 16 μl (0.0019 mmol) of the Traut's Reagent solution so obtained was added to the stirred solution of OVA with continued stirring. After 1 hour, non-reacted Traut's Reagent was removed using a centrifugal size exclusion column to afford the product of Formula 103′.

6A. Formula 103′ where X′ is Ovalbumin and m is 10
6B. Formula 902″ where X′ is Ovalbumin, m is 10 and n is 45

Dibenzocyclooctyne-PEG-(pyridyl disulfide) (Formula 901 where n is 45) (6.0 mg, 0.00238 mmol) was dissolved in DMF and the resulting solution was added to the OVA solution obtained in Example 6A and stirred for 1 hour. The excess dibenzocyclooctyne-PEG-(pyridyl disulfide) was removed using centrifugal size exclusion chromatography to afford the product of Formula 902″.

6C. Formula 1j where X′ is Ovalbumin, m is 10, n is 45, p is 55, q is 4. R⁸ is CH₂, R⁹ is a Direct Bond and Z″ is 2NAcGAL

Poly(Galactosamine Methacrylate)-N3 (Formula 803 where p is 55, q is 4 and Z″ is N-acetylgalactosamine) (36 mg, 0.00238 mmol) was dissolved in 150 μl of pH 7.4 PBS and added to the product of Example 6B with stirring. After 1 hour, the resulting product of Formula 1j was purified (excess p (GMA)-N3 removed) by centrifugal size exclusion chromatography. Characterization (UHPLC SEC, gel electrophoresis) confirmed the identity of the product.

### Example 7

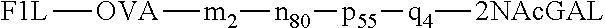

7A. Formula 1002 where X′ is Ovalbumin, m is 2 and n is 80

Dibenzocyclooctyne-PEG-(pyridyl disulfide) (Formula 1001 where n is 80) (9.0 mg, 0.00238 mmol) was dissolved in DMF and the resulting solution was added to a purified OVA solution of Formula 103′ (where X′ is Ovalbumin and m is 2), for example prepared as described in Example 6A and stirred for 1 hour. The excess dibenzocyclooctyne-PEG-(pyridyl disulfide) was removed using centrifugal size exclusion chromatography to afford the product of Formula 1002.

7B. Formula 1L where X′ is Ovalbumin, m is 2, n is 80, p is 55, q is 4, R⁸ is CH₂, R⁹ is a Direct Bond and Z″ is 2NAcGAL

Poly(Galactosamine Methacrylate)-N3 (Formula 803 where p is 55, q is 4 and Z″ is N-Acetylgalactosamine) (36 mg, 0.00238 mmol) was dissolved in 150 μl of pH 7.4 PBS and added to the product of Example 7A with stirring. After 1 hour, the resulting product of Formula 1L was purified (excess poly(Galactosamine Methacrylate)-N3 removed) by centrifugal size exclusion chromatography. Characterization (UHPLC SEC, gel electrophoresis) confirmed the identity of the product.

### Example 8

### Preparation of Poly(Galactosamine Methacrylate) Polymers

8A. Galactosamine Methacrylate

To stirred galactosamine hydrochloride (2.15 g, 10.0 mmol) was added 0.5 M sodium methoxide (22 ml, 11.0 mmol). After 30 minutes, methacrylate anhydride (14.694 g, 11.0 mmol) was added and stirring continued for 4 hours. The resulting galactosamine methacrylate was loaded onto silica gel via rotovap and purified via column chromatography using DCM:MeOH (85:15).

8B. Formula 201 where n is 4 and p is 30

Galactose methacrylate (600 mg, 2.43 mmol), 2-(2-(2-(2-(pyridin-2-yldisulfanyl) ethoxy) ethoxy) ethoxy)ethyl 2-((phenylcarbonothioyl)thio)acetate (44.8 mg, 0.081 mmol) and AIBN (3.174089069 mg, 0.016 mmol) were added to 1.5 ml of DMF in a Schlenk Flask. The reaction mixture was subjected to 4 freeze-thaw cycles and then stirred at 70° C. for 6 hours. The desired polymer product of Formula 201 was precipitated in 12 ml of methanol, and excess solvent was removed under reduced pressure.

### Example 9

Preparation of F1aA-PE-m₃-n₈₀
9A. Formula 103′ where X′ is Phycoerythrin

In an endotoxin-free tube, phycoerythrin (“PE”) (purchased from Pierce) (200 μl, 0.000004 mmol) was added to 50 μl of pH 8.0 PBS containing 5 mM EDTA and stirred. Separately, 1 mg of Taut's Reagent was dissolved in 100 μl of pH 7.0 PBS, and 2 μl (0.00013 mmol) of the Traut's Reagent solution so obtained was added to the stirred solution of PE with continued stirring. After 1 hour, excess Traut's Reagent was removed using a centrifugal size exclusion column to afford the product of Formula 103′.

9B. Formula 106A where n is 80

In an endotoxin-free tube, galactosamine (7.0 mg, 0.03246 mmol) was dissolved with stirring in 100 μl of pH 8.0 PBS containing 5 mM EDTA. Pyridyl dithiol-poly(ethylene glycol)-NHS ester (Formula 104 where n is 80) (16.23 mg, 0.00464 mmol) dissolved in 50 μl of pH 7.0 PBS was added to the stirring solution of galactosamine. After 1 hour, the resulting product of Formula 106A was ready to be used without further purification.

9C. Formula 1a where X′ is Phycoerythrin, m is 3, n is 80 and Z′ is Galactosamine

The purified PE-Traut conjugates prepared in Example 9A were added directly to the stirring product of Formula 106A prepared in Example 9B. After 1 hour, the resulting product of Formula 1a was purified by passing the reaction mixture through a centrifugal size exclusion column. Characterization (UHPLC SEC, gel electrophoresis) confirmed the identity of the product.

### Example 10

### OVA-DOM

The mammalian cell expression vector pSecTag A was purchased from Life Technologies. The gene encoding the anti-ASGPR domain antibody, Dom26h-196-61, herein referred to as “DOM”, was purchased as a codon-optimized sequence for protein expression in human cells, from the provider Genscript. Sequences encoding OVA, DOM, the flexible linker Gly₃Ser, and the 6×His tag were cloned into mammalian expression vector pSecTag A, C-terminal to the Ig κ-chain secretion leader sequence, by site-directed mutagenesis, following a variation of the QuikChange protocol (Geiser, et al.)

### 10A. Preparation of Expression Vector

10B. Expression and Purification of Formula 2 where m′ is 1, m″ is 0, X is Ovalbumin, Y is Gly₃Ser, Z is Anti-ASGPR Dom26h-196-61

HEK293 cells were transiently transfected with modified pSecTag A vectors, prepared for example as described in Example 10A, using polyethylenimine. Transfected cells were cultured in FreeStyle 293 medium (Life Technologies) supplemented with valproic acid for 7 days, after which the cells were removed by centrifugation and the culture supernatants were collected and sterilized by filtration. The OVA/DOM fusion proteins of Formula 2 were purified from the culture supernatants by immobilized metal ion affinity chromatography using a HisTrap Ni²⁺ sepharose column (GE Healthcare), followed by size exclusion chromatography using a Superdex 75 column (GE Healthcare), having the following generalized structure:

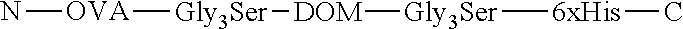

and the following amino acid sequence:

(SEQ ID NO: 28)
GSIGAASMEFCFDVFKELKVHHANENIFYCPIAIMSALAMVYLGAK
DSTRTQINKVVRFDKLPGFGDSIEAQCGTSVNVHSSLRDILNQITK
PNDVYSFSLASRLYAEERYPILPEYLQCVKELYRGGLEPINFQTAA
DQARELINSWVESQTNGIIRNVLQPSSVDSQTAMVLVNAIVFKGLW
EKAFKDEDTQAMPFRVTEQESKPVQMMYQIGLFRVASMASEKMKIL
ELPFASGTMSMLVLLPDEVSGLEQLESIINFEKLTEWTSSNVMEER
KIKVYLPRMKMEEKYNLTSVLMAMGITDVFSSSANLSGISSAESLK
ISQAVHAAHAEINEAGREVVGSAEAGVDAASVSEEFRADHPFLFCI
KHIATNAVLFFGRCVSPGGGSEVQLLESGGGLVQPGGSLRLSCAAS
GFTFEKYAMAWVRQAPGKGLEWVSRISARGVTTYYADSVKGRFTIS
RDNSKNTLYLQMNSLRAEDTAVYYCASHKRHEHTRFDSWGQGTLVT
VSSGGGSHHHHHH

Protein purity was verified by Coomassie Brilliant Blue staining of SDS-PAGE gels and anti-6×His tag western blotting. Protein concentration was determined using the Beer-Lambert Law, for which the absorbance at 280 nm was measured using a NanoDrop 2000 (Thermo Scientific), and the molecular weight (57.3 kDa) and extinction coefficient (57,090 M⁻¹ cm⁻¹) were estimated from the protein's amino acid sequence, using the ExPASy ProtParam tool. Endotoxin levels were measured using the HEK-BLUE TLR reporter cell line (Invivogen) according to manufacturer's instructions.

### 10C. Other Compositions of Formula 2

By following the procedures described in Examples 10A and 10B and substituting the pSecTag A vectors accordingly, there were obtained the following fusion proteins of Formula 2:

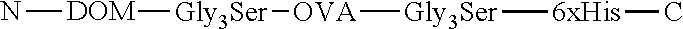

having the following amino acid sequence:

(SEQ ID NO: 29)
EVQLLESGGGLVQPGGSLRLSCAASGFTFEKYAMAWVRQAPGKGLE
WVSRISARGVTTYYADSVKGRFTISRDNSKNTLYLQMNSLRAEDTA
VYYCASHKRHEHTRFDSWGQGTLVTVSSGGGSGSIGAASMEFCFDV
FKELKVHHANENIFYCPIAIMSALAMVYLGAKDSTRTQINKVVRFD
KLPGFGDSIEAQCGTSVNVHSSLRDILNQITKPNDVYSFSLASRLY
AEERYPILPEYLQCVKELYRGGLEPINFQTAADQARELINSWVESQ
TNGIRNVLQPSSVDSQTAMVLVNAIVFKGLWEKAFKDEDTQAMPFR
VTEQESKPVQMMYQIGLFRVASMASEKMKILELPFASGTMSMLVLL
PDEVSGLEQLESIINFEKLTEWTSSNVMEERKIKVYLPRMKMEEKY
NLTSVLMAMGITDVFSSSANLSGISSAESLKISQAVHAAHAEINEA
GREVVGSAEAGVDAASVSEEFRADHPFLFCIKHIATNAVLFFGRCV
SPGGGSHHHHHH;
and
N-OVA-Gly₃Ser-DOM-Gly₃Ser-OVA-Gly₃Ser-6xHis-C

having the following amino acid sequence:

(SEQ ID NO: 30)
GSIGAASMEFCFDVFKELKVHHANENIFYCPIAIMSALAMVYLGAK
DSTRTQINKVVRFDKLPGFGDSIEAQCGTSVNVHSSLRDILNQITK
PNDVYSFSLASRLYAEERYPILPEYLQCVKELYRGGLEPINFQTAA
DQARELINSWVESQTNGIIRNVLQPSSVDSQTAMVLVNAIVFKGLW
EKAFKDEDTQAMPFRVTEQESKPVQMMYQIGLFRVASMASEKMKIL
ELPFASGTMSMLVLLPDEVSGLEQLESIINFEKLTEWTSSNVMEER
KIKVYLPRMKMEEKYNLTSVLMAMGITDVFSSSANLSGISSAESLK
ISQAVHAAHAEINEAGREVVGSAEAGVDAASVSEEFRADHPFLFCI
KHIATNAVLFFGRCVSPGGGSEVQLLESGGGLVQPGGSLRLSCAAS
GFTFEKYAMAWVRQAPGKGLEWVSRISARGVTTYYADSVKGRFTIS
RDNSKNTLYLQMNSLRAEDTAVYYCASHKRHEHTRFDSWGQGTLVT
VSSGGGSGSIGAASMEFCFDVFKELKVHHANENIFYCPIAIMSALA
MVYLGAKDSTRTQINKVVRFDKLPGFGDSIEAQCGTSVNVHSSLRD
ILNQITKPNDVYSFSLASRLYAEERYPILPEYLQCVKELYRGGLEP
INFQTAADQARELINSWVESQTNGIIRNVLQPSSVDSQTAMVLVNA
IVFKGLWEKAFKDEDTQAMPFRVTEQESKPVQMMYQIGLFRVASMA
SEKMKILELPFASGTMSMLVLLPDEVSGLEQLESIINFEKLTEWTS
SNVMEERKIKVYLPRMKMEEKYNLTSVLMAMGITDVFSSSANLSGI
SSAESLKISQAVHAAHAEINEAGREVVGSAEAGVDAASVSEEFRAD
HPFLFCIKHIATNAVLFFGRCVSPGGGSHHHHHH.

### 10D. Other Compositions of Formula 2

By following the procedures described in Examples 10A and 10B and substituting for Gly₃Ser the vectors for a linker having an immunoproteasome cleavage site (“IPC”), and for OVA the vectors for:
MBP13-32 (SEQ ID NO:11),
MBP83-99 (SEQ ID NO:12),
MBP111-129 (SEQ ID NO:13),
MBP146-170 (SEQ ID NO:14),
MOG1-20 (SEQ ID NO:15),
MOG35-55 (SEQ ID NO:16), and
PLP139-154 (SEQ ID NO:17),

### Or the Vectors for:

Alpha-gliadin “33-mer” deamidated (SEQ ID NO:25)
Alpha-gliadin (SEQ ID NO:26), and
Omega-gliadin (SEQ ID NO:27), there are obtained the following fusion proteins of Formula 2:
MBP13-32-IPC-MBP83-99-IPC-MBP111-129-IPC-MBP146-170-IPC-MOG1-20-IPC-MOG35-55-IPC-PLP139-154-IPC-DOM, and
Alpha-gliadin “33-mer” deamidated-IPC-Alpha-gliadin-IPC-Omega-gliadin-IPC-DOM.

### Example 11

### Desialylated OVA

### 11A. Preparation of Expression Vector

The mammalian cell expression vector pSecTag A was purchased from Life Technologies. Sequences encoding OVA, the flexible linker Gly3Ser, and the 6×His tag were cloned into pSecTag A, C-terminal to the Ig κ-chain secretion leader sequence, by site-directed mutagenesis, following a variation of the QuikChange protocol (Geiser, et al.)

### 11B. Expression and Purification of OVA

HEK293 cells were transiently transfected with modified pSecTag A vector, prepared for example as described in Example 10A, using polyethylenimine. Transfected cells were cultured in FreeStyle 293 medium (Life Technologies) supplemented with valproic acid for 7 days, after which the cells were removed by centrifugation and the culture supernatants are collected and sterilized by filtration. OVA protein was purified from the culture supernatant by immobilized metal ion affinity chromatography using a HisTrap Ni2+ sepharose column (GE Healthcare), followed by size exclusion chromatography using a Superdex 75 column (GE Healthcare), having the following structure:

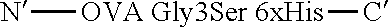

and the following amino acid sequence:

(SEQ ID NO: 31)
GSIGAASMEFCFDVFKELKVHHANENIFYCPIAIMSALAMVYLGAK
DSTRTQINKVVRFDKLPGFGDSIEAQCGTSVNVHSSLRDILNQITK
PNDVYSFSLASRLYAEERYPILPEYLQCVKELYRGGLEPINFQTAA
DQARELINSWVESQTNGIIRNVLQPSSVDSQTAMVLVNAIVFKGLW
EKAFKDEDTQAMPFRVTEQESKPVQMMYQIGLFRVASMASEKMKIL
ELPFASGTMSMLVLLPDEVSGLEQLESIINFEKLTEWTSSNVMEER
KIKVYLPRMKMEEKYNLTSVLMAMGITDVFSSSANLSGISSAESLK
ISQAVHAAHAEINEAGREVVGSAEAGVDAASVSEEFRADHPFLFCI
KHIATNAVLFFGRCVSPGGGSHHHHHH

Protein purity was verified by Coomassie Brilliant Blue staining of SDS-PAGE gels and anti-6×His tag western blotting. Protein concentration was determined using the Beer-Lambert Law, for which the absorbance at 280 nm was measured using a NanoDrop 2000 (Thermo Scientific), and the molecular weight (43.8 kDa) and extinction coefficient (31,525 M−1 cm−1) were estimated from the protein's amino acid sequence, using the ExPASy ProtParam tool.

### 11C. Desialylation

OVA is desialylated by incubation with neuraminidase for 1 hour at 37° C. (New England Biolabs). Desialylated OVA is purified from the reaction mixture by immobilized metal ion affinity chromatography (e.g., using a HisTrap Ni2+ sepharose column, GE Healthcare), followed by size exclusion chromatography (e.g., using a Superdex 75 column, GE Healthcare).

Protein purity is verified by Coomassie Brilliant Blue staining of SDS-PAGE gels and anti-6×His tag western blotting. Desialylation is verified by Sambucus nigra lectin-based detection of protein sialic acid content in western blots (Vector Biolabs) and by a sialic acid-mediated fluorescence assay (ProZyme). Desialylated protein concentration is determined using the Beer-Lambert Law, as described above for the protein before desialylation. Endotoxin levels are measured using the HEK-Blue TLR4 reporter cell line (Invivogen) according to manufacturer's instructions.

### Example 12

### Hepatic Distribution

12A. F1aA-PE-m₃-n₈₀ was prepared, for example, as described in Example 9. A 30 μg/100 μl solution in sterile saline was prepared for injection.

The F1aA-PE-m₃-n₈₀ solution (30 μg) was administered to one of three groups of C57 black 6 mice 3 per group) via tail vein injection. The two other groups of mice received an equivalent volume of phycoerythrin in 100 μl of saline or saline vehicle. Three hours after administration, the livers and spleens of these animals were harvested and the level of cellular fluorescents in these organs was determined by flow cytometry as an indication of cellular PE content.

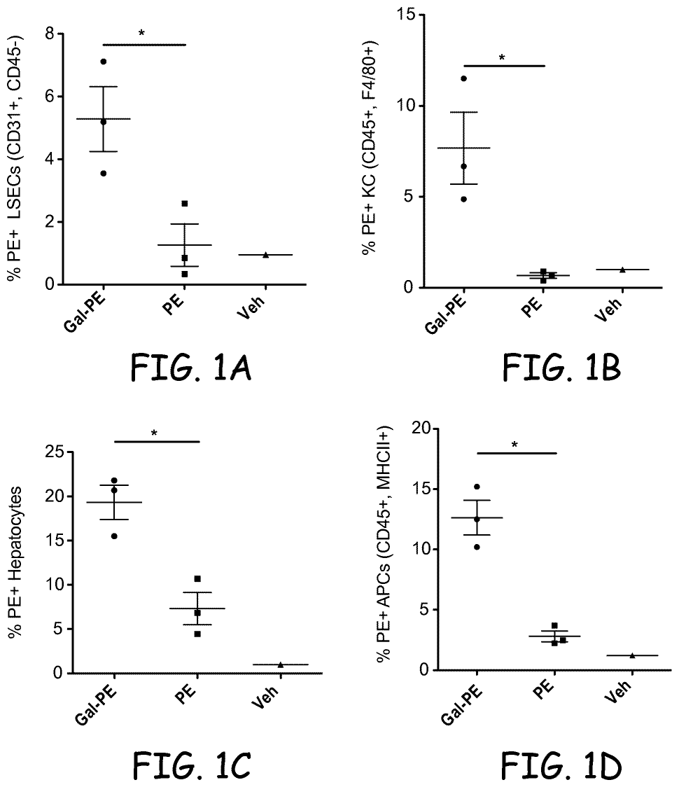

As shown in FIG. 1A-1D, sinusoidal endothelial cells (LSECs), hepatocytes, kupffer cells (KC), and other antigen-presenting cells (APCs) from the livers of mice treated with F1aA-PE-m₃-n₈₀ exhibited at least a three-fold increase in fluorescence as compared with animals that received PE solution. No detectible difference in fluorescence was found in spleen cells harvested from the three groups. These results confirm that F1aA-PE-m₃-n₈₀ has sufficient specificity for binding to antigen-presenting cells in the liver.

12B. By following the procedure described in Example 12A and substituting F1aA-PE-m₃-n₈₀ with the compounds F1b-PE-m₃-n₄-p₃₄-2NAcGAL, F1f-PE-m₃-n₄-p₃₃₋₂NAcGAL, F1g-PE-m₃-p₉₀-2NAcGAL, F1h-PE-m₃-n₄₅-p₅₅-q₄-2NAcGAL, F1j-PE-m₃-n₄₅-p₅₅-q₄-2NAcGAL, F1L-PE-m₃-n₈₀-p₅₅-q₄-2NAcGAL, F1m-PE-m₃-n₈₀-p₃₀-q₄-CMP-2NHAc, F1m-PE-m₃-n₆₂-p₃₀-q₈-CMP-2OH, F1n-PE-m₃-n₁-p₃₀-q₄-CMP-2NHAc and F1n-PE-m₃-n₃₃-p₃₀-q₈-CMP-2OH, prepared, for example, as described with reference to Example 9 by substitution for X in Examples 2B, 3, 4, 5B, 6B, 7B, 19G, 19L, 20B and 20F, respectively it is confirmed that the compounds F1aA-PE-m₃-n₈₀ with the compounds F1b-PE-m₃-n₄-p₃₄-2NAcGAL, F1f-PE-m₃-n₄-p₃₃-2NAcGAL, F1g-PE-m₃-p₉₀-2NAcGAL, F1h-PE-m₃-45-p₅₅-q₄-2NAcGAL, F1j-PE-m₃-n₄₅-p₅₅-q₄-2NAcGAL, F1L-PE-m₃-n₈₀-p₅₅-q₄-2NAcGAL, F1m-PE-m₃-n₈₀-p₃₀-q₄-CMP-2NHAc, F1m-PE-m₃-n₆₂-p₃₀-q₈-CMP-2OH, F1n-PE-m₃-n₁-p₃₀-q₄-CMP-2NHAc and F1n-PE-m₃-n₃₃-p₃₀-q₈-CMP-2OH have sufficient specificity for binding to antigen-presenting cells in the liver.

### Example 13

### Proliferation of Antigen-Specific OT1 CD8+ Tcells

13A. F1aA-OVA-m₄-n₈₀ synthesized, for example, as described in Example 1, was prepared as a 10 μg/100 μl saline solution for injection. On day 0, 106 OT-1 T cells were fluorescently labeled and adoptively transferred into 3 groups of CD 45.2 mice (5 per group) via tail vein injection. The next day (i.e. Day 1), to each of the 3 groups of mice were administered, respectively, 10 μg of F1aA-OVA-m₄-n₈₀, OVA or saline via tail vein injection. On day 6, the animals were sacrificed and the % of splenic proliferating OT-1 cells was determined via florescence activated cell sorting.

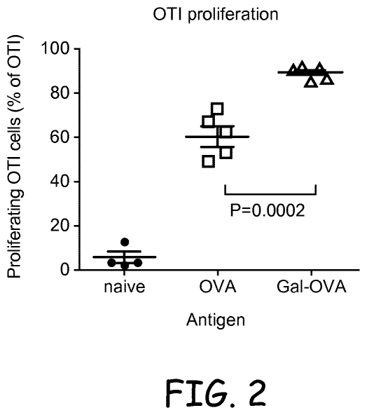

The results from this study (see FIG. 2) show that the percentage of proliferating OTI T cells in mice treated with F1aA-OVA-m₄-n₈₀ (“Gal-OVA” in FIG. 2) was significantly greater than the percentage of proliferating OTI cells in the spleens of mice treated with OVA or saline (“naïve” in FIG. 2). The increase in OTI cell-proliferation demonstrates the increased CD8+ T-cell cross-priming in animals treated with F1aA-OVA-m₄-80 versus the other therapies. In concert with the results from Example 12, these results indicate that the ability of F1aA-OVA-m₄-n₈₀ to target antigens to the liver increases OVA presentation by antigen presenting cells in the liver to OVA-specific OTI T cells.

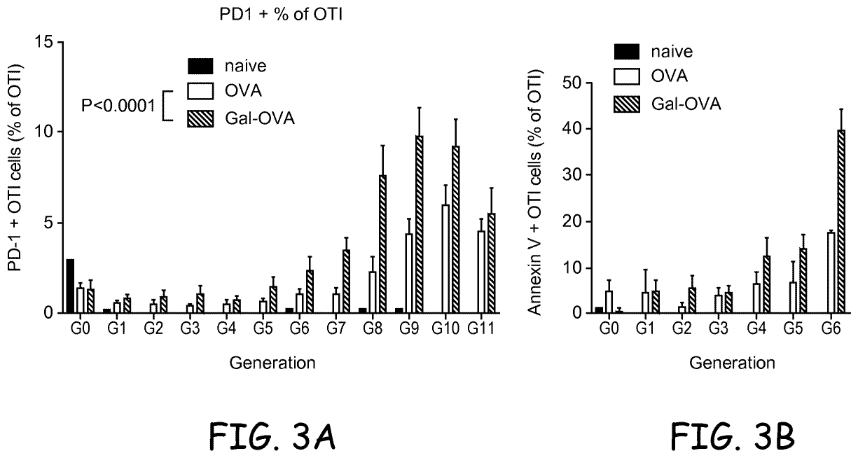

13B. To distinguish T cells being expanded into a functional effector phenotype from those being expanded and deleted, the proliferating OTI CD8+ T cells were analyzed for annexin-V, as a hallmark of apoptosis and thus deletion, as well as the exhaustion marker programmed death-1 (PD-1). As shown in FIG. 3A-3B, F1aA-OVA-m₄-n₈₀ (“Gal-OVA” in FIG. 3A-3B) induced much higher numbers of annexin-V⁺ and PD-1⁺ proliferating OTI CD8⁺ T cells than soluble OVA.

13C. By following the procedure described in Examples 13A and 13B, and substituting F1aA-OVA-m₄-n₈ with the compounds of Formula 1 obtained, for example, as described in Examples 3A, 4A, 5B, 6C, 7B and 19G, it is shown the compounds from Examples 3A, 4A, 5B, 6C, 7B and 19G induce much higher numbers of annexin-V⁺ and PD-1⁺ proliferating OTI CD8⁺ T cells than soluble OVA.

13D. By following the procedure described in Examples 13A and 13B and substituting F1aA-OVA-m₄-ns with the compounds of Formulae 1 and 2 obtained, for example, as described in Examples 1E, 1G, 2C, 10D, 191, 19L, 20B, 20D and 20F, and substituting OVA with the antigens corresponding to X (or X′ or X″), respectively, it is shown that the compounds from Examples 1E, 1G, 2C, 10D, 191, 19L, 20B, 20D and 20F induce much higher numbers of annexin-V⁺ and PD-1⁺ proliferating OTI CD8⁺ T cells than soluble antigen X.

### Example 14

F1aA-OVA-m₄-Ng does not Induce an OVA-Specific Antibody Response

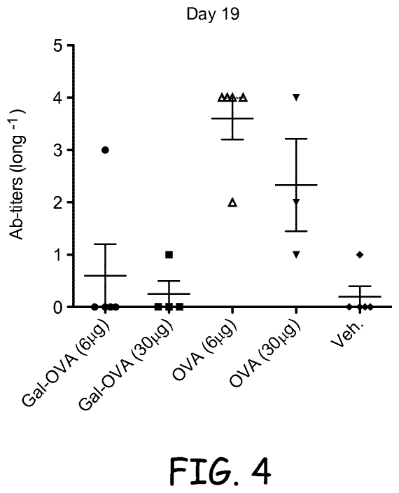

14A. In order to assess the humoral immune response to F1aA-OVA-m₄-ns we treated mice with a weekly i.v. injection of either F1aA-OVA-m₄-ns or OVA, then measured the levels of OVA-specific antibodies in the blood. On day 0, 7, and 14 of the experiment, mice were administered an i.v. injection of 100 μl of saline containing one of the following: 1.) 6 μg of OVA; 2.) 6 μg of F1aA-OVA-m₄-n₈; 3.) 30 μg of OVA; 4.) 30 μg of F1aA-OVA-m₄-n₈, or 5.) saline alone. Each group contained 5 mice. On day 19, the mice were bled via cheek puncture, and the titer of OVA-specific antibodies in each mouse's blood was determined via ELISA. The results for this study show that although mice treated with 6 and 30 μg of OVA had increased OVA-specific antibody titers, mice treated with both 6 and 30 μg of F1aA-OVA-m₄-n₈ (“Gal-OVA” in FIG. 4) had blood titers similar to mice treated with saline (i.e. vehicle treated animals) (FIG. 4). For example mice treated with 6 and 30 μg of OVA had an average antibody titer of 3.5 and 2.5, respectively; whereas, mice treated with 6 and 30 μg of OVA had an average antibody titer of 0.75 and 0.25, respectively.

14B. By following the procedure described in Example 14A and substituting F1aA-OVA-m₄-n₈ with the compounds of Formula 1 obtained, for example, as described in Examples 3A, 4A, 5B, 6C, 7B and 19G, it is shown that mice treated with the compounds from Examples 3A, 4A, 5B, 6C, 7B and 19G have OVA-specific antibody titers similar to mice treated with saline.

14C. By following the procedure described in Example 14A and substituting F1aA-OVA-m₄-n₈ with the compounds of Formulae 1 and 2 obtained, for example, as described in Examples 1E, 1G, 2C, 10D, 191, 19L, 20B, 20D and 20F, and substituting OVA with the antigens corresponding to X (or X′ or X″), respectively, it is shown that mice treated with the compounds from Examples 1E, 1G, 2C, 10D, 191, 19L, 20B, 20D and 20F have antigen X-specific antibody titers similar to mice treated with saline.

### Example 15

### F1aA-OVA-m₄-Ng Depletes OVA-Specific Antibodies

15A. We treated mice that had different OVA-antibody blood titers (each mouse had a titer from 0 to 4.5) with an i.v. injection of 20 μg of F1aA-OVA-m₄-ns solubilized in 100 μl saline. Mice were given i.v. injections of F1aA-OVA-m₄-ns on days 0, 5, 7, 12, and 14 (Injections of F1aA-OVA-m₄-ns are labeled as “Gal-OVA” and shown as green arrows on the x-axis of FIG. 5). In order to determine the ability of F1aA-OVA-m₄-ns to deplete serum OVA-specific antibodies, the mice were bled on day-1 to establish an initial antibody titer and then subsequent bleeds were carried out after each injection of F1aA-OVA-m₄-n₈ on days 2, 6, 9. 13, and 16. The antibody titer for each mouse was determined via ELISA. The results from this study show that F1aA-OVA-m₄-ns is able to deplete serum antibody levels in mice. For example, one day after the first F1aA-OVA-m₄-ns injection (i.e. day 2), mice with positive OVA-antibody titers experience a 5 to 100-fold decrease in serum antibody levels (FIG. 5). Our results show that although over the course of the 19 day experiment, antibody titers did increase for certain mice, the titer levels never reached the initial antibody titer measured on Day-1 and subsequent doses of F1aA-OVA-m₄-n₈ were effective in reducing these transient increases in antibody titers. These results demonstrate that F1aA-OVA-m₄-ns has the specificity to bind serum OVA-specific antibodies and the kinetics required to deplete OVA-specific serum antibodies.

15B. By following the procedure described in Example 15A and substituting F1aA-OVA-m₄-n₈ with the compounds of Formula 1 obtained, for example, as described in Examples 3A, 4A, 5B, 6C, 7B and 19G, it is shown that the compounds from Examples 3A, 4A, 5B, 6C, 7B and 19G have the specificity to bind serum OVA-specific antibodies and the kinetics required to deplete OVA-specific serum antibodies.

15C. By following the procedure described in Example 15A and substituting F1aA-OVA-m₄-n₈ with the compounds of Formulae 1 and 2 obtained, for example, as described in Examples 1E, 1G, 2C, 10D, 191, 19L, 20B, 20D and 20F, and substituting OVA with the antigens corresponding to X (or X′ or X″), respectively, it is shown that the compounds from Examples 1E, 1G, 2C, 10D, 191, 19L, 20B, 20D and 20F have the specificity to bind serum antigen X-specific antibodies and the kinetics required to deplete antigen X-specific serum antibodies.

### Example 16

### OT-1 Challenge-to-Tolerance Model

16A. Using an established OTI challenge-to-tolerance model (Liu, lyoda, et al., 2002), the ability of F1aA-OVA-m₄-n₈ (mGal-OVA), F1b-OVA-m₁-n₄-p₃₄ (pGal-OVA), and N-DOM-Gly₃Ser-OVA-Gly₃Ser-6×His-C (Dom-OVA) to prevent subsequent immune responses to vaccine-mediated antigen challenge were demonstrated-even with a challenge involving a very strong bacterially-derived adjuvant (i.e. lipopolysaccharide). To tolerize, 233 nmol of either F1aA-OVA-m₄-n₈, F1b-OVA-m₁-n₄-p₃₄, N-DOM-Gly₃Ser-OVA-Gly₃Ser-6×His-C, or soluble OVA were intravenously administered in 100 μl saline at 1 and 6 days following adoptive transfer of OTI CD8+ (CD45.2+) T cells to CD45.1+ mice (n=5 mice per group). After 9 additional days to allow potential deletion of the transferred T cells, the recipient mice were then challenged with OVA (10 μg) adjuvanted with lipopolysaccharide (LPS) (50 ng) by intradermal injection. Characterization of the draining lymph nodes 4 d after challenge allowed a determination as to whether or not deletion actually took place.

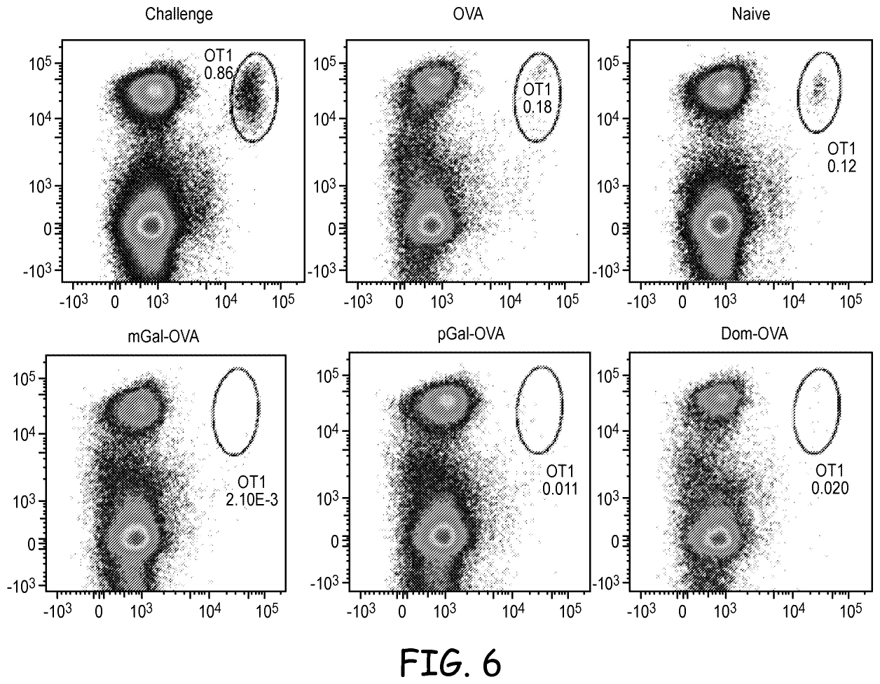

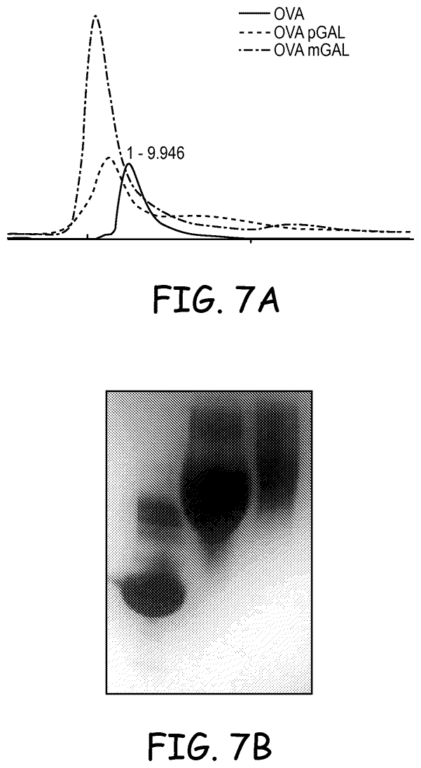

16B. Intravenous administration of F1aA-OVA-m₄-n₈, F1b-OVA-m₁-n₄-p₃₄, and N-DOM-Gly₃Ser-OVA-Gly₃Ser-6×His-C resulted in profound reductions in OTI CD8+ T cell populations in the draining lymph nodes as compared to mice treated with unmodified OVA prior to antigen challenge with LPS, demonstrating deletional tolerance. For example, FIG. 6 shows that the draining lymph nodes from mice treated with either F1aA-OVA-m₄-8 (mGal-OVA), F1b-OVA-m₁-n₄-p₃₄ (pGal-OVA), and N-DOM-Gly₃Ser-OVA-Gly₃Ser-6×His-C (Dom-OVA) contained over 9-fold fewer OTI CD8+ T cells as compared to OVA-treated mice, and more than 43-fold fewer than the challenge control mice that did not receive intravenous injections of antigen; responses in spleen cells were similar. These results demonstrate that F1aA-OVA-m₄-n₈, F1b-OVA-m₁-n₄-p₃₄, and N-DOM-Gly₃Ser-OVA-Gly₃Ser-6×His-C mitigated an OVA-specific immune response after adjuvanted OVA challenge.

16C. By following the procedure described in Examples 16A and B, and substituting F1aA-OVA-m₄-n₈, F1b-OVA-m₁-n₄-p₃₄, and N-DOM-Gly₃Ser-OVA-Gly₃Ser-6×His-C with the compounds of Formula 1 obtained, for example, as described in Examples 3A, 4A, 5B, 6C, 7B and 19G, it is shown that the compounds from Examples 3A, 4A, 5B, 6C, 7B and 19G mitigate an OVA-specific immune response after adjuvanted OVA challenge.

16D. By following the procedure described in Examples 16A and B, and substituting F1aA-OVA-m₄-n₈, F1b-OVA-m₁-n₄-p₃₄, and N-DOM-Gly₃Ser-OVA-Gly₃Ser-6×His-C with the compounds of Formulae 1 and 2 obtained, for example, as described in Examples 1E, 1G, 2C, 10D, 191, 19L, 20B, 20D and 20F, and substituting OVA with the antigens corresponding to X (or X′ or X″), respectively, it is shown that the compounds from Examples 1E, 1G, 2C, 10D, 191, 19L, 20B, 20D and 20F mitigate an antigen X-specific immune response after adjuvanted antigen X challenge.

### Example 17

### Pharmacokinetics

17A. OVA and fusion proteins N-DOM-Gly₃Ser-OVA-Gly₃Ser-6×His-C and N-OVA-Gly₃Ser-DOM-Gly₃Ser-6×His-C (prepared, e.g., as described in Example 10) are labeled with IRDye 800CW (LI-COR Biosciences) according to manufacturer's instructions. Unreacted dye is removed using Zeba desalting columns (Thermo Scientific). The labeled proteins, 50 μg in 100 μl PBS per dose, are administered i.v. or s.c into C57BL/6 mice (5 mice per group). At time=0, 0.25, 0.5, 1, 2, 4, 8, 24, 48, and 96 hours after injection, blood samples are collected from the tip of the tail into heparin-coated capillary tubes. Samples are store at 4° C., protected from light, until analysis.

On the day of analysis, blood samples are centrifuged to remove cellular components. Plasma is transferred to fresh capillary tubes and scanned using an Odyssey Infrared Imaging System (LI-COR Biosciences). Signal is acquired in the 800 nm channel. Using the image-processing program ImageJ (US National Institutes of Health), each sample is approximated as a line of width 2 and the mean intensity along the line is determined as a relative measure of the amount of circulating protein at that time point.

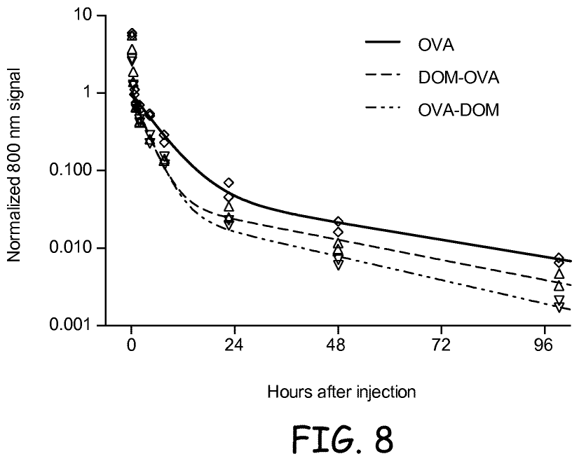

Fluorescence signals (normalized to fluorescence at time=0) vs. time for OVA and fusion proteins DOM-OVA and OVA-DOM, along with curve fits in the form of bi-exponential decays, in FIG. 8, show that the ASGPR-targeted OVA fusion proteins are cleared from circulation more quickly than unmodified OVA.

17B. By following the procedure described in Example 17A and substituting N-DOM-Gly₃Ser-OVA-Gly₃Ser-6×His-C fusion protein and OVA with asialo-Factor VIII and Factor VIII, respectively, it is shown that asialo-Factor VIII is cleared from circulation much more quickly than unmodified Factor VIII.

### Example 18

### Biodistribution

18A. OVA and N-DOM-Gly₃Ser-OVA-Gly₃Ser-6×His-C fusion protein (prepared, e.g., as described in Example 10) are labeled with Alexa Fluor 647 (Life Technologies) according to manufacturer's instructions. Unreacted dye is removed using Zeba desalting columns (Thermo Scientific). Labeled proteins, 50 μg in 100 μl PBS per dose, are administered i.v. or s.c into C57BL/6 mice (5 mice per group). Two hours after injection, mice are euthanized by CO₂ asphyxiation. The blood, heart, intestines, kidneys, liver, lungs, stomach, spleen, and remaining carcass are imaged using an IVIS Spectrum imaging system (Caliper Life Sciences). Data is acquired and analyzed using Living Image software (Caliper Life Sciences). Single cell suspensions of liver and spleen are prepared and stained with fluorescently conjugated antibodies against MHC class II, CD1d, CD3, CD4, CD8a, CD11b, CD11c, CD14, CD19, CD45, CD123, and/or Lin in PBS with 0.1% (w/v) BSA. Samples are analyzed using a LSR II flow cytometer and FACS Diva software (BD Biosciences).

A histogram showing fluorescence signal densities (photons/weight) for each organ and each protein shows that in animals injected with N-DOM-Gly₃Ser-OVA-Gly₃Ser-6×His-C fusion protein the highest fluorescence signal is in the liver as compared to those treated with unmodified OVA.

A histogram showing percentages of N-DOM-Gly₃Ser-OVA-Gly₃Ser-6×His-C and OVA-positive cells shows that animals injected with N-DOM-Gly₃Ser-OVA-Gly₃Ser-6×His-C fusion proteins have a significantly higher percentage of positive hepatocytes as compared to those treated with unmodified OVA.

18B. By following the procedure described in Example 18A and substituting N-DOM-Gly₃Ser-OVA-Gly₃Ser-6×His-C fusion protein and OVA with asialo-Factor VIII and Factor VIII, respectively, it is shown that animals injected with asialo-Factor VIII have a significantly higher percentage of positive hepatocytes as compared to those treated with unmodified Factor VIII.

18C. By following the procedure described in Example 18A and substituting N-DOM-Gly₃Ser-OVA-Gly₃Ser-6×His-C fusion protein with the compounds of Formula 1 obtained, for example, as described in Examples 3A, 4A, 5B, 6C, 7B and 19G, it is shown that animals injected with the compounds from Examples 3A, 4A, 5B, 6C, 7B and 19G have a significantly higher percentage of positive hepatocytes as compared to those treated with OVA.

18D. By following the procedure described in Examples 18A and B, and substituting F1aA-OVA-m₄-n₈, F1b-OVA-m₁-n₄-p₃₄, and N-DOM-Gly₃Ser-OVA-Gly₃Ser-6×His-C with the compounds of Formulae 1 and 2 obtained, for example, as described in Examples 1E, 1G, 2C, 10D, 191, 19L, 20B, 20D and 20F, and substituting OVA with the antigens corresponding to X (or X′ or X″), respectively, it is shown that it is shown that animals injected with the compounds from Examples 1E, 1G, 2C, 10D, 191, 19L, 20B, 20D and 20F have a significantly higher percentage of positive hepatocytes as compared to those treated with antigen X.

### Example 19

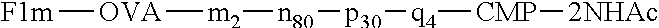

19A. Formula 1102 where R³ is NHAc and R⁴ is OH

N-Acetyl-D-galactosamine (Formula 1101 where R³ is NHAc and R⁴ is OH) (5 g, 22.6 mmol) was added to a stirred solution of chloroethanol (200 ml) at room temperature. The solution was cooled to 4° C. and acetylchloride was added drop-wise to the solution. The solution was brought to room temperature and then heated to 70° C. After 4 hours, the unreacted choroethanol was removed under reduced pressure. 100 ml of ethanol was added to the crude product and the resulting solution was stirred in the presence of carbon for 2 hours. The solution was filtered, and the solvent was removed under reduced pressure. The corresponding product of Formula 1102, N-(2-(2-chloroethoxy)-4,5-dihydroxy-6-(hydroxymethyl)tetrahydro-2H-pyran-3-yl) acetamide, was used without further purification.

19B. Formula 1103 where R³ is NHAc and R⁴ is OH

The N-(2-(2-chloroethoxy)-4,5-dihydroxy-6-(hydroxymethyl)tetrahydro-2H-pyran-3-yl) acetamide prepared in Example 19A (2 g, 7.4 mmol) was added to a stirred solution of DMF (100 ml) and sodium azide (4 g, 61.5 mmol). The solution was headed at 90° C. for 12 hours and then filtered. The residual solvent was removed under reduced pressure and the crude product was purified via flash chromatography (10% MeOH in dichloromethane) to give the corresponding product of Formula 1103, N-(2-(2-azidoethoxy)-4,5-dihydroxy-6-(hydroxymethyl)tetrahydro-2H-pyran-3-yl) acetamide.

19C. Formula 1104 where R³ is NHAc and R⁴ is OH

The N-(2-(2-azidoethoxy)-4,5-dihydroxy-6-(hydroxymethyl)tetrahydro-2H-pyran-3-yl) acetamide prepared in Example 19B (2 g, 6.9 mmol) was added to a solution of palladium on carbon and ethanol (50 ml). The solution was stirred under hydrogen gas (3 atm) for 4 hours. The resulting solution was filtered and the residual solvent was removed under reduced pressure to afford the corresponding product of Formula 1104, N-(2-(2-aminoethoxy)-4,5-dihydroxy-6-(hydroxymethyl)tetrahydro-2H-pyran-3-yl) acetamide, which was used without further purification.

19D. Formula 1105 where R³ is NHAc and R⁴ is OH

The N-(2-(2-aminoethoxy)-4,5-dihydroxy-6-(hydroxymethyl)tetrahydro-2H-pyran-3-yl) acetamide prepared in Example 19C (1.0 g, 3.78 mmol) was added to a solution of methacrylate anhydride (0.583 g, 3.78 mmol) in DMF (50 m₁). Triethylamine was then added to the solution and the reaction was stirred for 2 hours at room temperature. After 2 hours, the excess solvent was removed under reduced pressure, and the corresponding product of Formula 1105, N-(2-((3-acetamido-4,5-dihydroxy-6-(hydroxymethyl)tetrahydro-2H-pyran-2-yl)oxy)ethyl) methacrylamide, was isolated via flash chromatography.

19E. Formula 1107 where p is 30, q is 4, R³ is NHAc. R⁴ is OH and R⁸ is CMP

An azide-modified uRAFT agent of Formula 1106 where q is 4 (28 mg) was added to a solution of N-(2-((3-acetamido-4,5-dihydroxy-6-(hydroxymethyl)tetrahydro-2H-pyran-2-yl)oxy)ethyl) methacrylamide prepared in Example 19D (579 mg, 1.74 mmol) and azobisisobutyronitrile (2.2 mg, 0.0116 mmol) in DMF. The reaction mixture was subjected to 4 free-pump-thaw cycles, and then stirred at 70° C. After 12 hours, the polymer product of Formula 1107, where p is 30 and q is 4 was precipitated from the reaction mixture via the addition of methanol. The solvent was decanted from the solid and the solid was collected and residual solvent was removed via reduced pressure.

19F. Formula 1109 where X′ is OVA, m is 2 and n is 80

Ovalbumin (5 mg, 0.00012 mmol) was added to 100 μl of sodium phosphate buffer (pH 8.0) and stirred. To this solution was added 5 mg of the compound of Formula 1108 where n is 80. After 1 hour, the unreacted compound of Formula 1108 was removed from the solution via centrifugal size-exclusion chromatography. The resulting buffered solution containing the corresponding product of Formula 1109 was used in the next reaction without further purification.

19G. Formula 1m where X′ is OVA, m is 2, n is 80, p is 30, q is 4, R³ is NHAc and R⁸ is CMP

The solution prepared in Example 19F was added to 100 μl of sodium phosphate buffer (pH 8.0) which contained 10 mg of the product of Formula 1107 prepared in Example 19E. The reaction was allowed to stir for 2 hours and then the excess Formula 1107 was removed via centrifugal size exclusion chromatography to afford the corresponding isomeric product of Formula 1m in solution, which was used in biological studies without further purification. The R³ substituent is shown in the name of the title compound as 2NHAc.

### 19H. Other Compounds of Formula 1109

By following the procedure described in Example 19F and substituting OVA with the following:
Abciximab,
Adalimumab,
Agalsidase alfa,
Agalsidase beta,
Aldeslukin,
Alglucosidase alfa,
Factor VIII,
Factor IX,
L-asparaginase,
Laronidase,
Octreotide,
Phenylalanine ammonia-lyase,
Rasburicase,
Insulin (SEQ ID NO:5),
GAD-65 (SEQ ID NO:6),
IGRP (SEQ ID NO:7)
MBP (SEQ ID NO:8),
MOG (SEQ ID NO:9),
PLP (SEQ ID NO:10),
MBP13-32 (SEQ ID NO:11),
MBP83-99 (SEQ ID NO:12),
MBP111-129 (SEQ ID NO:13),
MBP146-170 (SEQ ID NO:14),
MOG1-20 (SEQ ID NO:15),
MOG35-55 (SEQ ID NO:16),
PLP139-154 (SEQ ID NO:17),
MART1 (SEQ ID NO:18),
Tyrosinase (SEQ ID NO:19),
PMEL (SEQ ID NO:20),
Aquaporin-4 (SEQ ID NO:21),
S-arrestin (SEQ ID NO:22),
IRBP (SEQ ID NO:23),
Conarachin (UNIPROT Q6PSU6),
Alpha-gliadin “33-mer” native (SEQ ID NO:24),
Alpha-gliadin “33-mer” deamidated (SEQ ID NO:25),
Alpha-gliadin (SEQ ID NO:26),
Omega-gliadin (SEQ ID NO:27),
Fel d 1A (UNIPROT P30438),
Cat albumin (UNIPROT P49064),
Can f 1 (UNIPROT O18873),
Dog albumin (UNIPROT P49822), and
RhCE example (UNIPROT P18577), there are obtained the following corresponding compounds of Formula 1109 where n is 80:
X is Abciximab and m is 10,
X is Adalimumab and m is 11,
X is Agalsidase alfa and m is 14,
X is Agalsidase beta and m is 14,
X is Aldeslukin and m is 6,
X is Alglucosidase alfa and m is 13,
X is Factor VIII and m is 100,
X is Factor IX and m is 18,
X is L-asparaginase and m is 5,
X is Laronidase and m is 7,
X is Octreotide and m is 1,
X is Phenylalanine ammonia-lyase and m is 12,
X is Rasburicase and m is 12,
X is Insulin (SEQ ID NO:5) and m is 2,
X is GAD-65 (SEQ ID NO:6) and m is 8,
X is IGRP (SEQ ID NO:7) and m is 7,
X is MBP (SEQ ID NO:8) and m is 6,
X is MOG (SEQ ID NO:9) and m is 5,
X is PLP (SEQ ID NO:10) and m is 8,
X is MBP13-32 (SEQ ID NO:11) and m is 1,
X is MBP83-99 (SEQ ID NO: 12) and m is 1,
X is MBP111-129 (SEQ ID NO:13) and m is 1,
X is MBP146-170 (SEQ ID NO:14) and m is 2,
X is MOG1-20 (SEQ ID NO:15) and m is 1,
X is MOG35-55 (SEQ ID NO:16) and m is 2,
X is PLP139-154 (SEQ ID NO:17) and m is 3,
X is MART1 (SEQ ID NO:18) and m is 4,
X is Tyrosinase (SEQ ID NO:19) and m is 8,
X is PMEL (SEQ ID NO:20) and m is 5,
X is Aquaporin-4 (SEQ ID NO:21) and m is 4,
X is S-arrestin (SEQ ID NO:22) and m is 12,
X is IRBP (SEQ ID NO:23) and m is 21,
X is Conarachin and m is 21,
X is Alpha-gliadin “33-mer” native (SEQ ID NO:24) and m is 1,
X is Alpha-gliadin “33-mer” deamidated (SEQ ID NO:25) and m is 1,
X is Alpha-gliadin (SEQ ID NO:26) and m is 1,
X is Omega-gliadin (SEQ ID NO:27) and m is 1,
X is Fel d 1 and m is 4,
X is Cat albumin and m is 16,
X is Can f 1 and m is 6,
X is Dog albumin and m is 23, and
X is RhCE example and m is 10.

### 191. Other Compounds of Formula 1m

By following the procedure described in Example 19G and substituting the compounds of Formula 1109, for example as obtained in Example 19H, there are obtained the following corresponding compounds of Formula 1m:
F1m-Abciximab-m₁₀-n₈₀-p₃₀-q₄-CMP-2NHAc,
F1m-Adalimumab-m₁₁-n₈₀-p₃₀-q₄-CMP-2NHAc,
F1m-Agalsidase alfa-m₁₄-n₈₀-p₃₀-q₄-CMP-2NHAc,
F1m-Agalsidase beta-m₁₄-n₈₀-p₃₀-q₄-CMP-2NHAc,
F1m-Aldeslukin-m₆-n₈₀-p₃₀-q₄-CMP-2NHAc,
F1m-Alglucosidase alfa-m₁₃-n₈₀-p₃₀-q₄-CMP-2NHAc,
F1m-Factor VIII-m₁₀₀-n₈₀-p₃₀-q₄-CMP-2NHAc,
F1m-Factor IX-m₁₈-n₈₀-p₃₀-q₄-CMP-2NHAc,
F1m-L-asparaginase-m₅-n₈₀-p₃₀-q₄-CMP-2NHAc,
F1m-Laronidase-m₇-n₈₀-p₃₀-q₄-CMP-2NHAc,
F1m-Octreotide-m₁-n₈₀-p₃₀-q₄-CMP-2NHAc,
F1m-Phenylalanine ammonia-lyase-m₁₂-n₈₀-p₃₀-q₄-CMP-2NHAc,
F1m-Rasburicase-m₁₂-n₈₀-p₃₀-q₄-CMP-2NHAc,
F1m-Insulin-m₂-n₈₀-p₃₀-q₄-CMP-2NHAc,
F1m-GAD-65-m₈-n₈₀-p₃₀-q₄-CMP-2NHAc,
F1m-IGRP-m₇-n₈₀-p₃₀-q₄-CMP-2NHAc,
F1m-MBP-m₆-n₈₀-p₃₀-q₄-CMP-2NHAc,
F1m-MOG-m₅-n₈₀-p₃₀-q₄-CMP-2NHAc,
F1m-PLP-m₈-n₈₀-p₃₀-q₄-CMP-2NHAc,
F1m-MBP13-32-m₁-n₈₀-p₃₀-q₄-CMP-2NHAc,
F1m-MBP83-99-m₁-n₈₀-p₃₀-q₄-CMP-2NHAc,
F1m-MBP111-129-m₁-n₈₀-p₃₀-q₄-CMP-2NHAc,
F1m-MBP146-170-m₂-n₈₀-p₃₀-q₄-CMP-2NHAc,
F1m-MOG1-20-m₁-n₈₀-p₃₀-q₄-CMP-2NHAc,
F1m-MOG35-55-m₂-n₈₀-p₃₀-q₄-CMP-2NHAc,
F1m-PLP139-154-m₃-n₈₀-p₃₀-q₄-CMP-2NHAc,
F1m-MART1-m₄-n₈₀-p₃₀-q₄-CMP-2NHAc,
F1m-Tyrosinase-m₈-n₈₀-p₃₀-q₄-CMP-2NHAc,
F1m-PMEL-m₅-n₈₀-p₃₀-q₄-CMP-2NHAc,
F1m-Aquaporin-4-m₄-n₈₀-p₃₀-q₄-CMP-2NHAc,
F1m-S-arrestin-m₁₂-n₈₀-p₃₀-q₄-CMP-2NHAc,
F1m-IRBP-m₂₁-n₈₀-p₃₀-q₄-CMP-2NHAc,
F1m-Conarachin-m₂₁-n₈₀-p₃₀-q₄-CMP-2NHAc,
F1m-Alpha-gliadin “33-mer” native-m₁-n₈₀-p₃₀-q₄-CMP-2NHAc,
F1m-Alpha-gliadin “33-mer” deamidated-m₁-n₈₀-p₃₀-q₄-CMP-2NHAc,
F1m-Alpha-gliadin-m₁-n₈₀-p₃₀-q₄-CMP-2NHAc,
F1m-Omega-gliadin-m₁-n₈₀-p₃₀-q₄-CMP-2NHAc,
F1m-Fel d 1-m₄-n₈₀-p₃₀-q₄-CMP-2NHAc,
F1m-Cat albumin-m₁₆-n₈₀-p₃₀-q₄-CMP-2NHAc,
F1m-Can f 1-m₆-n₈₀-p₃₀-q₄-CMP-2NHAc,
F1m-Dog albumin-m₂₃-n₈₀-p₃₀-q₄-CMP-2NHAc, and
F1m-RhCE-m₁₀-n₈₀-p₃₀-q₄-CMP-2NHAc.
   19J. Formula 1107 where p is 30, q is 8, R³ is OH, R⁴ is OH and R⁸ is CMP

By following the procedure described in Example 19A and substituting the N-acetyl-D-galactosamine with galactose, and following through to the procedure described in Example 19E except using an azide-modified uRAFT agent of Formula 1106 where q is 8, there is obtained the compound of Formula 1107 where p is 30, q is 8, R³ is OH, R⁴ is OH and R⁸ is CMP.

19K. Formula 1109 where n is 62 and where X′ and m are as in Example 19H

By following the procedure described in Example 19F, substituting the OVA with the compounds as described in Example 19H and employing the compound of Formula 1108 where n is 62, there are obtained the corresponding compounds of Formula 1109 where n is 62.

### 19L. Other Compounds of Formula 1m

By following the procedure described in Example 19G and substituting the compound of Formula 1107 with the compounds obtained in Example 19J, and substituting the compound of Formula 1109 with the compounds obtained in Example 19K, there are obtained the following corresponding compounds of Formula 1m:
F1m-Abciximab-m₁₀-n₆₂-p₃₀-q₈-CMP-2OH,
F1m-Adalimumab-m₁₁-n₆₂-p₃₀-q₈-CMP-2OH,
F1m-Agalsidase alfa-m₁₄-n₆₂-p₃₀-q₈-CMP-2OH,
F1m-Agalsidase beta-m₁₄-n₆₂-p₃₀-q₈-CMP-2OH,
F1m-Aldeslukin-m₆-n₆₂-p₃₀-q₈-CMP-2OH,
F1m-Alglucosidase alfa-m₁₃-n₆₂-p₃₀-q₈-CMP-2OH,
F1m-Factor VIII-m₁₀₀-n₆₂-p₃₀-q₈-CMP-2OH,
F1m-Factor IX-m₁₈-n₆₂-p₃₀-q₈-CMP-2OH,
F1m-L-asparaginase-m₅-n₆₂-p₃₀-q₈-CMP-2OH,
F1m-Laronidase-m₇-n₆₂-p₃₀-q₈-CMP-2OH,
F1m-Octreotide-m₁-n₆₂-p₃₀-q₈-CMP-2OH,
F1m-Phenylalanine ammonia-lyase-m₁₂-n₆₂-p₃₀-q₈-CMP-2OH,
F1m-Rasburicase-m₁₂-n₆₂-p₃₀-q₈-CMP-2OH,
F1m-Insulin-m₂-n₆₂-p₃₀-q₈-CMP-2OH,
F1m-GAD-65-m₈-n₆₂-p₃₀-q₈-CMP-2OH,
F1m-IGRP-m₇-n₆₂-p₃₀-q₈-CMP-2OH,
F1m-MBP-m₆-n₆₂-p₃₀-q₈-CMP-2OH,
F1m-MOG-m₅-n₆₂-p₃₀-q₈-CMP-2OH,
F1m-PLP-m₈-n₆₂-p₃₀-q₈-CMP-2OH,
F1m-MBP13-32-m₁-n₆₂-p₃₀-q₈-CMP-2OH,
F1m-MBP83-99-m₁-n₆₂-p₃₀-q₈-CMP-2OH,
F1m-MBP111-129-m₁-n₆₂-p₃₀-q₈-CMP-2OH,
F1m-MBP146-170-m₂-n₆₂-p₃₀-q₈-CMP-2OH,
F1m-MOG1-20-m₁-n₆₂-p₃₀-q₈-CMP-2OH,
F1m-MOG35-55-m₂-n₆₂-p₃₀-q₈-CMP-2OH,
F1m-PLP139-154-m₃-n₆₂-p₃₀-q₈-CMP-2OH,
F1m-MART1-m₄-n₆₂-p₃₀-q₈-CMP-2OH,
F1m-Tyrosinase-m₈-n₆₂-p₃₀-q₈-CMP-2OH,
F1m-PMEL-m₅-n₆₂-p₃₀-q₈-CMP-2OH,
F1m-Aquaporin-4-m₄-n₆₂-p₃₀-q₈-CMP-2OH,
F1m-S-arrestin-m₁₂-n₆₂-p₃₀-q₈-CMP-2OH,
F1m-IRBP-m₂₁-n₆₂-p₃₀-q₈-CMP-2OH,
F1m-Conarachin-m₂₁-n₆₂-p₃₀-q₈-CMP-2OH,
F1m-Alpha-gliadin “33-mer” native-m₁-n₆₂-p₃₀-q₈-CMP-2OH,
F1m-Alpha-gliadin “33-mer” deamidated-m₁-n₆₂-p₃₀-q₈-CMP-2OH,
F1m-Alpha-gliadin-m₁-n₆₂-p₃₀-q₈-CMP-2OH,
F1m-Omega-gliadin-m₁-n₆₂-p₃₀-q₈-CMP-2OH,
F1m-Fel d 1-m₄-n₆₂-p₃₀-q₈-CMP-2OH,
F1m-Cat albumin-m₁₆-n₆₂-p₃₀-q₈-CMP-2OH,
F1m-Can f 1-m₆-n₆₂-p₃₀-q₈-CMP-2OH,
F1m-Dog albumin-m₂₃-n₆₂-p₃₀-q₈-CMP-2OH, and
F1m-RhCE-m₁₀-n₆₂-p₃₀-q₈-CMP-2OH.

### Example 20

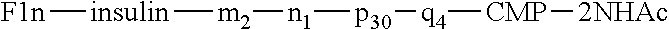

20A. Formula 1202 where X′ is Insulin, m is 2 and n is 1

Recombinant human insulin (5 mg) was added to 100 μl of DMF containing 10 μl of triethylamine and stirred until the insulin became soluble. To this solution was added 10 mg (0.0161 mmol) of a linker precursor of Formula 1201 where n is 1 and the reaction was allowed to stir. After 1 hour, 1.3 ml of tert-butyl methyl ether was added to isolate the corresponding product of Formula 1202, which was recovered as the precipitate. Residual DMF and tert-butyl methyl ether were removed under reduced pressure. Characterization via liquid chromatography, mass spectroscopy and polyacrylamide gel electrophoresis confirmed the identity of the product. The modified insulin product of Formula 1202 was used without further purification.

20B. Formula 1n where X′ is Insulin, m is 2, n is 1, p is 30, q is 4 and R⁸ is CMP

The product of Formula 1202 obtained in Example 20A was resuspended in 100 μl of DMF. The polymer product of Formula 1107 obtained in Example 19E (10 mg) was added and the reaction was allowed to stir for 1 hour. After 1 hour, the reaction products were precipitated via the addition of dichloromethane (1.3 ml). The product was filtered and the residual solvent was removed under reduced pressure. The crude product was then resuspended in 500 μl of PBS, and the low molecular weight components were removed via centrifugal size exclusion chromatography to afford the corresponding isomeric product of Formula 1n. Characterization via liquid chromatography, mass spectroscopy and polyacrylamide gel electrophoresis confirmed the identity of the product. The modified insulin product of Formula 1202 was used without further purification.

### 20C. Other Compounds of Formula 1202

By following the procedure described in Example 19F and substituting OVA insulin the following:
Abciximab,
Adalimumab,
Agalsidase alfa,
Agalsidase beta,
Aldeslukin,
Alglucosidase alfa,
Factor VIII,
Factor IX,
L-asparaginase,
Laronidase,
Octreotide,
Phenylalanine ammonia-lyase,
Rasburicase,
GAD-65 (SEQ ID NO:6),
IGRP (SEQ ID NO:7)
MBP (SEQ ID NO:8),
MOG (SEQ ID NO:9),
PLP (SEQ ID NO:10),
MBP13-32 (SEQ ID NO:11),
MBP83-99 (SEQ ID NO:12),
MBP111-129 (SEQ ID NO:13),
MBP146-170 (SEQ ID NO:14),
MOG1-20 (SEQ ID NO:15),
MOG35-55 (SEQ ID NO:16),
PLP139-154 (SEQ ID NO:17),
MART1 (SEQ ID NO:18),
Tyrosinase (SEQ ID NO:19),
PMEL (SEQ ID NO:20),
Aquaporin-4 (SEQ ID NO:21),
S-arrestin (SEQ ID NO:22),
IRBP (SEQ ID NO:23),
Conarachin (UNIPROT Q6PSU6),
Alpha-gliadin “33-mer” native (SEQ ID NO:24),
Alpha-gliadin “33-mer” deamidated (SEQ ID NO:25),
Alpha-gliadin (SEQ ID NO:26),
Omega-gliadin (SEQ ID NO:27),
Fel d 1A (UNIPROT P30438),
Cat albumin (UNIPROT P49064),
Can f 1 (UNIPROT O18873),
Dog albumin (UNIPROT P49822), and
RhCE example (UNIPROT P18577), there are obtained the following corresponding compounds of Formula 1202 where n is 1:
X is Abciximab and m is 10,
X is Adalimumab and m is 11,
X is Agalsidase alfa and m is 14,
X is Agalsidase beta and m is 14,
X is Aldeslukin and m is 6,
X is Alglucosidase alfa and m is 13,
X is Factor VIII and m is 100,
X is Factor IX and m is 18,
X is L-asparaginase and m is 5,
X is Laronidase and m is 7,
X is Octreotide and m is 1,
X is Phenylalanine ammonia-lyase and m is 12,
X is Rasburicase and m is 12,
X is GAD-65 (SEQ ID NO:6) and m is 8,
X is IGRP (SEQ ID NO:7) and m is 7,
X is MBP (SEQ ID NO:8) and m is 6,
X is MOG (SEQ ID NO:9) and m is 5,
X is PLP (SEQ ID NO:10) and m is 8,
X is MBP13-32 (SEQ ID NO:11) and m is 1,
X is MBP83-99 (SEQ ID NO:12) and m is 1,
X is MBP111-129 (SEQ ID NO:13) and m is 1,
X is MBP146-170 (SEQ ID NO:14) and m is 2,
X is MOG1-20 (SEQ ID NO:15) and m is 1,
X is MOG35-55 (SEQ ID NO:16) and m is 2,
X is PLP139-154 (SEQ ID NO:17) and m is 3,
X is MART1 (SEQ ID NO:18) and m is 4,
X is Tyrosinase (SEQ ID NO:19) and m is 8,
X is PMEL (SEQ ID NO:20) and m is 5,
X is Aquaporin-4 (SEQ ID NO:21) and m is 4,
X is S-arrestin (SEQ ID NO:22) and m is 12,
X is IRBP (SEQ ID NO:23) and m is 21,
X is Conarachin and m is 21,
X is Alpha-gliadin “33-mer” native (SEQ ID NO:24) and m is 1,
X is Alpha-gliadin “33-mer” deamidated (SEQ ID NO:25) and m is 1,
X is Alpha-gliadin (SEQ ID NO:26) and m is 1,
X is Omega-gliadin (SEQ ID NO:27) and m is 1,
X is Fel d 1 and m is 4,
X is Cat albumin and m is 16,
X is Can f 1 and m is 6,
X is Dog albumin and m is 23, and
X is RhCE example and m is 10.

### 20D. Other Compounds of Formula 1n

By following the procedure described in Example B and substituting the compounds of Formula 1202, for example as obtained in Example 20C, there are obtained the following corresponding compounds of Formula 1m:

F1n-Abciximab-m₁₀-n₁-p₃₀-q₄-CMP-2NHAc,
F1n-Adalimumab-m₁₁-n₁-p₃₀-q₄-CMP-2NHAc,
F1n-Agalsidase alfa-m₁₄-1-p₃₀-q₄-CMP-2NHAc,
F1n-Agalsidase beta-m₁₄-n₁-p₃₀-q₄-CMP-2NHAc,
F1n-Aldeslukin-m₆-n₁-p₃₀-q₄-CMP-2NHAc,
F1n-Alglucosidase alfa-m₁₃-n₁-p₃₀-q₄-CMP-2NHAc,
F1n-Factor VIII-m₁₀₀-n₁-p₃₀-q₄-CMP-2NHAc,
F1n-Factor IX-m₁₈-n₁-p₃₀-q₄-CMP-2NHAc,
F1n-L-asparaginase-m₅-n₁-p₃₀-q₄-CMP-2NHAc,
F1n-Laronidase-m₇-n₁-p₃₀-q₄-CMP-2NHAc,
F1n-Octreotide-m₁-n₁-p₃₀-q₄-CMP-2NHAc,
F1n-Phenylalanine ammonia-lyase-m₁₂-n₁-p₃₀-q₄-CMP-2NHAc,
F1n-Rasburicase-m₁₂-n₁-p₃₀-q₄-CMP-2NHAc,
F1n-GAD-65-m₈-n₁-p₃₀-q₄-CMP-2NHAc,
F1n-IGRP-m₇-n₁-p₃₀-q₄-CMP-2NHAc,
F1n-MBP-m₆-n₁-p₃₀-q₄-CMP-2NHAc,
F1n-MOG-ms-n₁-p₃₀-q₄-CMP-2NHAc,
F1n-PLP-m₈-n₁-p₃₀-q₄-CMP-2NHAc,
F1n-MBP13-32-m₁-n₁-p₃₀-q₄-CMP-2NHAc,
F1n-MBP83-99-m₁-1-p₃₀-q₄-CMP-2NHAc,
F1n-MBP111-129-m₁-n₁-p₃₀-q₄-CMP-2NHAc,
F1n-MBP146-170-m₂-n₁-p₃₀-q₄-CMP-2NHAc,
F1n-MOG1-20-m₁-n₁-p₃₀-q₄-CMP-2NHAc,
F1n-MOG35-55-m₂-n₁-p₃₀-q₄-CMP-2NHAc,
F1n-PLP139-154-m₃-1-p₃₀-q₄-CMP-2NHAc,
F1n-MART1-m₄-n₁-p₃₀-q₄-CMP-2NHAc,
F1n-Tyrosinase-m₈-n₁-p₃₀-q₄-CMP-2NHAc,
F1n-PMEL-m₅-n₁-p₃₀-q₄-CMP-2NHAc,
F1n-Aquaporin-4-m₄-n₁-p₃₀-q₄-CMP-2NHAc,
F1n-S-arrestin-m₁₂-n₁-p₃₀-q₄-CMP-2NHAc,
F1n-IRBP-m₂₁-n₁-p₃₀-q₄-CMP-2NHAc,
F1n-Conarachin-m₂₁-n₁-p₃₀-q₄-CMP-2NHAc,
F1n-Alpha-gliadin “33-mer” native-m₁-n₁-p₃₀-q₄-CMP-2NHAc,
F1n-Alpha-gliadin “33-mer” deamidated-m₁-n₁-p₃₀-q₄-CMP-2NHAc,
F1n-Alpha-gliadin-m₁-n₁-p₃₀-q₄-CMP-2NHAc,
F1n-Omega-gliadin-m₁-n₁-p₃₀-q₄-CMP-2NHAc,
F1n-Fel d 1-m₄-n₁-p₃₀-q₄-CMP-2NHAc,
F1n-Cat albumin-m₁₆-n₁-p₃₀-q₄-CMP-2NHAc,
F1n-Can f 1-m₆-n₁-p₃₀-q₄-CMP-2NHAc,
F1n-Dog albumin-m₂₃-n₁-p₃₀-q₄-CMP-2NHAc, and
F1n-RhCE-m₁₀-n₁-p₃₀-q₄-CMP-2NHAc.
   20E. Formula 1202 where n is 33 and where X′ and m are as in Example 20C

By following the procedure described in Example 19F, substituting the insulin with the compounds as described in Example 20C and employing the compound of Formula 1201 where n is 33, there are obtained the corresponding compounds of Formula 1202 where n is 33.

### 20F. Other Compounds of Formula 1n

By following the procedure described in Example 20B and substituting the compound of Formula 1107 with the compounds obtained in Example 19J, and substituting the compound of Formula 1202 with the compounds obtained in Example 20E, there are obtained the following corresponding compounds of Formula 1n:
F1n-Abciximab-m₁₀-n₃₃-p₃₀-q₈-CMP-2OH,
F1n-Adalimumab-m₁₁-n₃₃-p₃₀-q₈-CMP-2OH,
F1n-Agalsidase alfa-m₁₄-n₃₃-p₃₀-q₈-CMP-2OH,
F1n-Agalsidase beta-m₁₄-n₃₃-p₃₀-q₈-CMP-2OH,
F1n-Aldeslukin-m₆-n₃₃-p₃₀-q₈-CMP-2OH,
F1n-Alglucosidase alfa-m₁₃-n₃₃-p₃₀-q₈-CMP-2OH,
F1n-Factor VIII-m₁₀₀-n₃₃-p₃₀-q₈-CMP-2OH,
F1n-Factor IX-m₁₈-n₃₃-p₃₀-q₈-CMP-2OH,
F1n-L-asparaginase-m₅-n₃₃-p₃₀-q₈-CMP-2OH,
F1n-Laronidase-m₇-n₃₃-p₃₀-q₈-CMP-2OH,
F1n-Octreotide-m₁-n₃₃-p₃₀-q₈-CMP-2OH,
F1n-Phenylalanine ammonia-lyase-m₁₂-n₃₃-p₃₀-q₈-CMP-2OH,
F1n-Rasburicase-m₁₂-n₃₃-p₃₀-q₈-CMP-2OH,
F1n-GAD-65-m₈-n₃₃-p₃₀-q₈-CMP-2OH,
F1n-IGRP-m₇-n₃₃-p₃₀-q₈-CMP-2OH,
F1n-MBP-m₆-n₃₃-p₃₀-q₈-CMP-2OH,
F1n-MOG-m₅-n₃₃-p₃₀-q₈-CMP-2OH,
F1n-PLP-m₈-n₃₃-p₃₀-q₈-CMP-2OH,
F1n-MBP13-32-m₁-33-p₃₀-q₈-CMP-2OH,
F1n-MBP83-99-m₁-n₃₃-p₃₀-q₈-CMP-2OH,
F1n-MBP111-129-m₁-n₃₃-p₃₀-q₈-CMP-2OH,
F1n-MBP146-170-m₂-n₃₃-p₃₀-q₈-CMP-2OH,
F1n-MOG1-20-m₁-n₃₃-p₃₀-q₈-CMP-2OH,
F1n-MOG35-55-m₂-n₃₃-p₃₀-q₈-CMP-2OH,
F1n-PLP139-154-m₃-33-p₃₀-q₈-CMP-2OH,
F1n-MART1-m₄-n₃₃-p₃₀-q₈-CMP-2OH,
F1n-Tyrosinase-m₈-n₃₃-p₃₀-q₈-CMP-2OH,
F1n-PMEL-m₅-n₃₃-p₃₀-q₈-CMP-2OH,
F1n-Aquaporin-4-m₄-n₃₃-p₃₀-q₈-CMP-2OH,
F1n-S-arrestin-m₁₂-n₃₃-p₃₀-q₈-CMP-2OH,
F1n-IRBP-m₂₁-n₃₃-p₃₀-q₈-CMP-2OH,
F1n-Conarachin-m₂₁-n₃₃-p₃₀-q₈-CMP-2OH,
F1n-Alpha-gliadin “33-mer” native-m₁-n₃₃-p₃₀-q₈-CMP-2OH,
F1n-Alpha-gliadin “33-mer” deamidated-m₁-n₃₃-p₃₀-q₈-CMP-2OH,
F1n-Alpha-gliadin-m₁-n₃₃-p₃₀-q₈-CMP-2OH,
F1n-Omega-gliadin-m₁-n₃₃-p₃₀-q₈-CMP-2OH,
F1n-Fel d 1-m₄-n₃₃-p₃₀-q₈-CMP-2OH,
F1n-Cat albumin-m₁₆-n₃₃-p₃₀-q₈-CMP-2OH,
F1n-Can f 1-m₆-n₃₃-p₃₀-q₈-CMP-2OH,
F1n-Dog albumin-m₂₃-n₃₃-p₃₀-q₈-CMP-2OH, and
F1n-RhCE-m₁₀-n₃₃-p₃₀-q₈-CMP-2OH.

### Example 21

Clearance of OVA-Specific Antibodies from Circulation

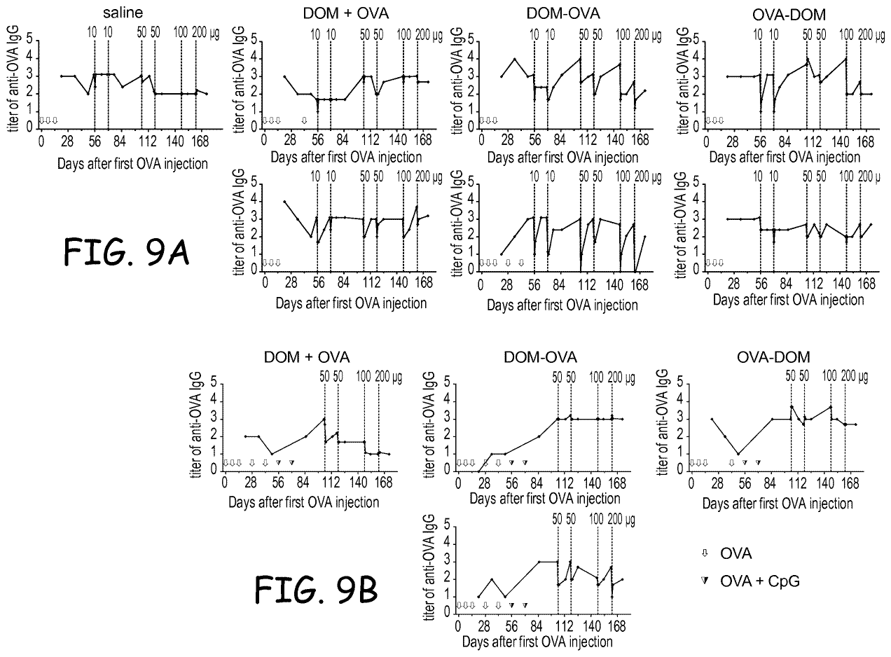

To induce production of OVA-specific antibodies for subsequent depletion, C57BL/6 mice were injected i.v. with up to five doses of 10 μg OVA until a titer of anti-OVA IgG in plasma, as determined by ELISA, reached 3 (FIG. 9A). Plasma samples were prepared by centrifugation of blood collected into EDTA-coated tubes at 2000× g for 10 minutes at room temperature and stored at −20° C. until analysis. Titer is defined as the log₁₀ of the maximal fold dilution of plasma with detectable anti-OVA IgG. Here, plasma samples were assayed at dilutions of 10-, 50-, 100-, 500-, 1,000-, 5,000-, 10,000-, and 50,000-fold, yielding potential titer measurements of 0, 1, 1.7, 2, 2.7, 3, 3.7, 4, and 4.7. Mice with titers below 3 after five doses of 10 μg OVA were given two additional doses of 10 μg OVA adjuvanted with 10 μg CpG-B to boost anti-OVA IgG production and hence the amount of circulating anti-OVA IgG available for depletion (FIG. 9B).

To evaluate the efficacy of hepatocyte ASGPR-targeted OVA for anti-OVA antibody clearance, mice with circulating anti-OVA IgG were injected i.v. with saline or saline containing molar equivalents of DOM and OVA, DOM-OVA, or OVA-DOM. In particular, mice were treated with molar equivalents of 10, 50, 100, or 200 μg OVA, as indicated in FIG. 9A-9B. At times t=−1 day, +3 hours, +1 day, and +7 days, relative to the time of treatment injection, blood samples were collected to assess, by ELISA analysis of plasma, the amount of anti-OVA IgG in circulation. Clearance of anti-OVA IgG from circulation is expected to decrease the amount of anti-OVA IgG in plasma and hence decrease plasma anti-OVA IgG titer as determined by ELISA. In mice immunized with OVA alone and treated with DOM-OVA or OVA-DOM, titers of anti-OVA IgG decreased by up to 3 or 2, respectively, representing decreases in the amount of circulating anti-OVA IgG by 1000- or 100-fold, which is up to 50-fold more effective than control treatment using saline or DOM and OVA (FIG. 9A). In mice immunized with OVA adjuvanted with CpG-B, clearance of circulating anti-OVA IgG was not observed (FIG. 9B).

### Example 22

### NOD Mouse

Non-obese diabetic (NOD) mice are susceptible to the spontaneous onset of autoimmune diabetes mellitus, which is the result of an autoimmune response to various pancreatic auto-antigens. Diabetes develops in NOD mice as a result of insulitis, characterized by the infiltration of various leukocytes into the pancreatic islets.

In order to evaluate the efficacy of a treatment for diabetes mellitus, starting at 6-weeks of age, NOD mice are divided into control or test groups and treated, respectively, with weekly intravenous injections of a test composition (10 μg) or an inactive control such as saline. The injections continue for 18 consecutive weeks.

The blood glucose concentration of the mice is measured weekly. Mice that maintain a blood glucose concentration of less than 300 mg/ml during the experiment are considered non-diabetic. In addition, at the end of the study the pancreases of the mice are harvested and T cell infiltration in the pancreas is determined via immunohistochemistry as an assessment of insulitis. Tolerance induction is assessed by the depletion of auto-antigen specific CD8 T cells as compared to mice that are treated with saline and develop diabetes. The existence of auto-antigen specific CD8 T cells is determined via ELISpot assay.

When tested as described above, compositions of Formulae 1 and 2 where X is insulin, such as F1m-insulin-m₂-n₁-p₃₀-q₄-2NAcGAL, show efficacy for treating diabetes mellitus.

While the present disclosure has been described with reference to the specific embodiments thereof, it should be understood by those skilled in the art that various changes can be made and equivalents can be substituted without departing from the true spirit and scope of the disclosure. In addition, many modifications can be made to adapt a particular situation, material, composition of matter, process, process step or steps, to the objective, spirit and scope of the present disclosure. All such modifications are intended to be within the scope of the claims appended hereto. All patents and publications cited above are hereby incorporated by reference.

What is claimed is:

1. A compound for the induction of antigen-specific immune tolerance in a subject, the compound comprising:
a peptide to which tolerance is desired;
wherein the peptide to which tolerance is desired, when presented to the subject alone, is capable of inducing an unwanted immune response in the subject;
a polymeric linker;
wherein the polymeric linker is coupled to the peptide to which tolerance is desired via a disulfide bond or a disulfanyl ethyl ester;
wherein the disulfide bond or the disulfanyl ethyl ester are each configured to be cleaved upon administration of the compound to the subject and to release the peptide to which tolerance is desired from the polymeric linker;
wherein the polymeric linker comprises a 1-cyano-1-methyl-propyl group and methacrylic units comprising an ethylacetamido functionality; and
a liver-targeting moiety;
wherein the liver-targeting moiety comprises a galactosylating moiety;
wherein the liver-targeting moiety is coupled to the polymeric linker through the ethylacetamido functionality.


SEQUENCE LISTING (35 sequences)

SEQ ID NO: 1 - protein, 6 aa, synthetic construct
  REGION 1..6: note = 6xHis
  REGION 1..6: note = MISC_FEATURE - His Tag or 6xHis
HHHHHH

SEQ ID NO: 2 - protein, 4 aa, synthetic construct
  REGION 1..4: note = Gly3Ser
  REGION 1..4: note = MISC_FEATURE - Gly3Ser
GGGS

SEQ ID NO: 3 - protein, 20 aa, Homo sapiens
  REGION 1..20: note = MISC_FEATURE - Ig Kappa Chain
METDTLLLWVLLLWVPGSTG

SEQ ID NO: 4 - protein, 22 aa, Erwinia carotovora
  REGION 1..22: note = MISC_FEATURE - pelB Signal Sequence
MKYLLPTAAAGLLLLAAQPAMA

SEQ ID NO: 5 - protein, 110 aa, Homo sapiens
  REGION 1..110: note = MISC_FEATURE - Human Insulin
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 6 - protein, 585 aa, Homo sapiens
  REGION 1..585: note = MISC_FEATURE - GAD-65
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 7 - protein, 355 aa, Homo sapiens
  REGION 1..355: note = MISC_FEATURE - IGRP
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 8 - protein, 304 aa, Homo sapiens
  REGION 1..304: note = MISC_FEATURE - Myelin Basic Protein
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 9 - protein, 247 aa, Homo sapiens
  REGION 1..247: note = MISC_FEATURE - Myelin Oligodendrocyte Protein
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 10 - protein, 277 aa, Homo sapiens
  REGION 1..277: note = MISC_FEATURE - Myelin Proteolipid Protein
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 11 - protein, 20 aa, Homo sapiens
  REGION 1..20: note = MISC_FEATURE - MBP 13-32
KYLATASTMDHARHGFLPRH

SEQ ID NO: 12 - protein, 16 aa, Homo sapiens
  REGION 1..16: note = MISC_FEATURE - MBP 83-99
ENPWHFFKNIVTPRTP

SEQ ID NO: 13 - protein, 19 aa, Homo sapiens
  REGION 1..19: note = MISC_FEATURE - MBP 111-129
LSRFSWGAEGQRPGFGYGG

SEQ ID NO: 14 - protein, 25 aa, Homo sapiens
  REGION 1..25: note = MISC_FEATURE - MBP 146-170
AQGTLSKIFKLGGRDSRSGSPMARR

SEQ ID NO: 15 - protein, 20 aa, Homo sapiens
  REGION 1..20: note = MISC_FEATURE - MOG 1-20
GQFRVIGPRHPIRALVGDEV

SEQ ID NO: 16 - protein, 20 aa, Homo sapiens
  REGION 1..20: note = MISC_FEATURE - MOG 35-55
MEVGWYRPPFSRWHLYRNGK

SEQ ID NO: 17 - protein, 16 aa, Homo sapiens
  REGION 1..16: note = MISC_FEATURE - PLP 139-154
HCLGKWLGHPDKFVGI

SEQ ID NO: 18 - protein, 118 aa, Homo sapiens
  REGION 1..118: note = MISC_FEATURE - MART1
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 19 - protein, 529 aa, Homo sapiens [sequence omitted]

SEQ ID NO: 20 - protein, 661 aa, Homo sapiens
  REGION 1..661: note = MISC_FEATURE - Melanocyte protein PMEL
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 21 - protein, 323 aa, Homo sapiens
  REGION 1..323: note = MISC_FEATURE - Aquaporin-4
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 22 - protein, 405 aa, Homo sapiens
  REGION 1..405: note = MISC_FEATURE - S-arrestin
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 23 - protein, 1247 aa, Homo sapiens
  REGION 1..1247: note = MISC_FEATURE - IRBP
MMREWVLLMSVLLCGLAGPTHLFQPSLVLDMAKVLLDNYCFPENLLGMQEAIQQAIKSHEILSISDPQTLASVLTAGVQSSLNDPRLVISYEPSTPEPPPQVPALTSLSEEELLAWLQRGLRHEVLEGNVGYLRVDSVPGQEVLSMMGEFLVAHVWGNLMGTSALVLDLRHCTGGQVSGIPYIISYLHPGNTILHVDTIYNRPSNTTTEIWTLPQVLGERYGADKDVVVLTSSQTRGVAEDIAHILKQMRRAIVVGERTGGGALDLRKLRIGESDFFFTVPVSRSLGPLGGGSQTWEGSGVLPCVGTPAEQALEKALAILTLRSALPGVVHCLQEVLKDYYTLVDRVPTLLQHLASMDFSTVVSEEDLVTKLNAGLQAASEDPRLLVRAIGPTETPSWPAPDAAAEDSPGVAPELPEDEAIRQALVDSVFQVSVLPGNVGYLRFDSFADASVLGVLAPYVLRQVWEPLQDTEHLIMDLRHNPGGPSSAVPLLLSYFQGPEAGPVHLFTTYDRRTNITQEHFSHMELPGPRYSTQRGVYLLTSHRTATAAEEFAFLMQSLGWATLVGEITAGNLLHTRTVPLLDTPEGSLALTVPVLTFIDNHGEAWLGGGVVPDAIVLAEEALDKAQEVLEFHQSLGALVEGTGHLLEAHYARPEVVGQTSALLRAKLAQGAYRTAVDLESLASQLTADLQEVSGDHRLLVFHSPGELVVEEAPPPPPAVPSPEELTYLIEALFKTEVLPGQLGYLRFDAMAELETVKAVGPQLVRLVWQQLVDTAALVIDLRYNPGSYSTAIPLLCSYFFEAEPRQHLYSVFDRATSKVTEVWTLPQVAGQRYGSHKDLYILMSHTSGSAAEAFAHTMQDLQRATVIGEPTAGGALSVGIYQVGSSPLYASMPTQMAMSATTGKAWDLAGVEPDITVPMSEALSIAQDIVALRAKVPTVLQTAGKLVADNYASAELGAKMATKLSGLQSRYSRVTSEVALAEILGADLQMLSGDPHLKAAHIPENAKDRIPGIVPMQIPSPEVFEELIKFSFHTNVLEDNIGYLRFDMFGDGELLTQVSRLLVEHIWKKIMHTDAMIIDMRFNIGGPTSSIPILCSYFFDEGPPVLLDKIYSRPDDSVSELWTHAQVVGERYGSKKSMVILTSSVTAGTAEEFTYIMKRLGRALVIGEVTSGGCQPPQTYHVDDTNLYLTIPTARSVGASDGSSWEGVGVTPHVVVPAEEALARAKEMLQHNQLRVKRSPGLQDHL

SEQ ID NO: 24 - protein, 33 aa, Triticum aestivum
  REGION 1..33: note = MISC_FEATURE - DQ-2 relevant, Alpha-gliadin "33-mer" native
LQLQPFPQPQLPYPQPQLPYPQPQLPYPQPQPF

SEQ ID NO: 25 - protein, 33 aa, Triticum aestivum
  REGION 1..33: note = MISC_FEATURE - DQ-2 relevant, Alpha-gliadin "33-mer" deamidated
LQLQPFPQPELPYPQPELPYPQPELPYPQPQPF

SEQ ID NO: 26 - protein, 18 aa, Triticum aestivum
  REGION 1..18: note = MISC_FEATURE - DQ-8 relevant, Alpha-gliadin
QQYPSGQGSFQPSQQNPQ

SEQ ID NO: 27 - protein, 12 aa, Triticum aestivum
  REGION 1..12: note = MISC_FEATURE - DQ-8 relevant, Omega-gliadin
QPFPQPEQPFPW

SEQ ID NO: 28 - protein, 519 aa, synthetic construct
  REGION 1..519: note = N- OVAGly3SerDOMGly3Ser 6xHis -C
  REGION 1..519: note = MISC_FEATURE - N- OVAGly3SerDOMGly3Ser 6xHis -C
GSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPGGGSEVQLLESGGGLVQPGGSLRLSCAASGFTFEKYAMAWVRQAPGKGLEWVSRISARGVTTYYADSVKGRFTISRDNSKNTLYLQMNSLRAEDTAVYYCASHKRHEHTRFDSWGQGTLVTVSSGGGSHHHHHH

SEQ ID NO: 29 - protein, 519 aa, synthetic construct
  REGION 1..519: note = N-DOM Gly3Ser OVA Gly3Ser 6xHis-C
  REGION 1..519: note = MISC_FEATURE - N-DOM Gly3Ser OVA Gly3Ser 6xHis-C
EVQLLESGGGLVQPGGSLRLSCAASGFTFEKYAMAWVRQAPGKGLEWVSRISARGVTTYYADSVKGRFTISRDNSKNTLYLQMNSLRAEDTAVYYCASHKRHEHTRFDSWGQGTLVTVSSGGGSGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPGGGSHHHHHH

SEQ ID NO: 30 - protein, 908 aa, synthetic construct
  REGION 1..908: note = N- OVA Gly3Ser DOM Gly3Ser OVA Gly3Ser 6xHis -C
  REGION 1..908: note = MISC_FEATURE - N- OVA Gly3Ser DOM Gly3Ser OVA Gly3Ser 6xHis -C
GSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPGGGSEVQLLESGGGLVQPGGSLRLSCAASGFTFEKYAMAWVRQAPGKGLEWVSRISARGVTTYYADSVKGRFTISRDNSKNTLYLQMNSLRAEDTAVYYCASHKRHEHTRFDSWGQGTLVTVSSGGGSGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPGGGSHHHHHH

SEQ ID NO: 31 - protein, 395 aa, synthetic construct
  REGION 1..395: note = N'- OVA Gly3Ser 6xHis -C'
  REGION 1..395: note = MISC_FEATURE - N'- OVA Gly3Ser 6xHis -C'
GSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPGGGSHHHHHH

SEQ ID NO: 32 - protein, 519 aa, synthetic construct; identical to SEQ ID NO: 29
  REGION 1..519: note = N- DOMGly3SerOVAC
  REGION 1..519: note = MISC_FEATURE - N- DOMGly3SerOVAC

SEQ ID NO: 33 - protein, 4 aa, synthetic construct
  REGION 1..4: note = Gly3Ser
  REGION 1..4: note = MISC_FEATURE - Gly3Ser
GGGS

SEQ ID NO: 34 - protein, 10 aa, synthetic construct
  REGION 1..10: note = Gly3Ser-6xHis
  REGION 1..10: note = MISC_FEATURE - Gly3Ser-6xHis
GGGSHHHHHH

SEQ ID NO: 35 - protein, 10 aa, synthetic construct
  REGION 1..10: note = 6xHisGly3Ser
  REGION 1..10: note = MISC_FEATURE - 6xHisGly3Ser
HHHHHHGGGS